(19) United States
(12) Patent Application Publication
(10) Pub. No.: US 2008/0249066 A1
(43) Pub. Date: Oct. 9, 2008

(54) ANTIVIRAL COMPOUNDS AND COMPOSITIONS

(75) Inventors: Barry D. Quart, Encinitas, CA (US); Zhi Hong, Chapel Hill, CA (US)

Correspondence Address: WILSON SONSINI GOODRICH & ROSATI, 650 PAGE MILL ROAD, PALO ALTO, CA, 94304-1050, US

(73) Assignee: ARDEA BIOSCIENCES, INC., San Diego, CA (US)

(21) Appl. No.: 12/054,306

(22) Filed: Mar. 24, 2008

Related U.S. Application Data
(60) Provisional application No. 60/896,849, filed on Mar. 23, 2007.
(60) Provisional application No. 60/968,241, filed on Aug. 27, 2007.
(60) Provisional application No. 60/972,672, filed on Sep. 14, 2007.

(51) Int. Cl. A61K 31/675; A61P 31/18

(52) U.S. Cl. 514/86

(57) ABSTRACT

Described herein are compounds useful in the treatment of viral diseases, compositions comprising them and methods of using them. The compounds comprise a nucleoside or nucleoside analog linked, commonly through a phosphate group to one of a selected group of lipids. In some embodiments, the compounds described herein are useful for treating HIV infection, AIDS and other viral infections.

### CROSS-REFERENCE

This application claims the benefit of U.S. Provisional Application No. 60/896,849, filed Mar. 23, 2007, U.S. Provisional Application No. 60/968,241, filed Aug. 27, 2007, and U.S. Provisional Application No. 60/972,672, filed Sep. 14, 2007, which applications are each incorporated herein in their entirety by reference.

### BACKGROUND OF THE INVENTION

Nucleoside analogues are useful for treating viral infections. For instance, 3′-azido-3′-deoxythymidine, or AZT is used for the treatment of HIV infections. However, many nucleoside analogs have many unwanted side effects. AZT's more severe side effects include anemia and bone marrow suppression. Additionally, in clinical situations, there is a rapid emergence of drug resistant variants of infection with an already resistant virus. These impair the chances for cure or for keeping the disease under control.

### SUMMARY OF THE INVENTION

In one aspect, the invention relates to compounds of formula (A-I) and pharmaceutically acceptable salts thereof:

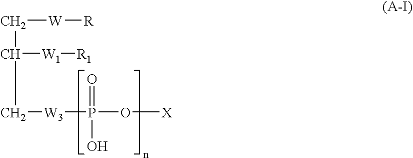

wherein
W is —S—, —S(O)—, —O—, —S—C═C—, —SC(O)—, —O—C═C— or —OC(O)—;
W₁ is —S—, —S(O)—, —O—, —S—C═C—, —SC(O)—, —O—C═C— or —OC(O)—;
W₃ is —O— or a covalent bond;
n is 1, 2 or 3;
R is C1-C7, C8-C12, C13-C18, or C19-C24 cyclic, straight-chained or branched, unsaturated or saturated alkyl which can be optionally substituted;
R₁ is C1, C2, C3-C10, C11-C15, C16-C17, or C18-C24 cyclic, straight-chained or branched, unsaturated or saturated alkyl which can be optionally substituted; and
X is a nucleoside or nucleoside analog radical.

In another aspect, the invention relates to compounds of formula (A-II) and pharmaceutically acceptable salts thereof:

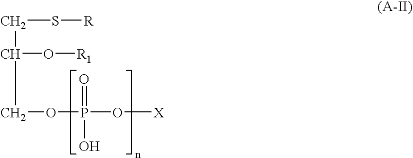

wherein
n is 1, 2 or 3;
R is C1-C7, C8-C12, C13-C18, or C19-C24 cyclic, straight-chained or branched, unsaturated or saturated alkyl which can be optionally substituted;
R₁ is C1, C2, C3-C10, C11-C15, C16-C17, or C18-C24 cyclic, straight-chained or branched, unsaturated or saturated alkyl which can be optionally substituted; and
X is a nucleoside or nucleoside analog radical.

In another aspect, the invention relates to compounds of formula (B-I) and pharmaceutically acceptable salts thereof:

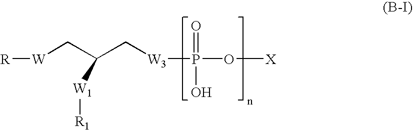

wherein
W is —S—, —S(O)—, —O—, —S—C═C—, —SC(O)—, —O—C═C— or —OC(O)—;
W₁ is —S—, —S(O)—, —O—, —S—C═C—, —SC(O)—, —O—C═C— or —OC(O)—;
W₃ is —O— or a covalent bond;
n is 1, 2 or 3;
R is optionally substituted, C1-C24, straight-chained or branched, unsaturated or saturated alkyl;
R₁ is optionally substituted C1-C24 straight-chained or branched, unsaturated or saturated alkyl; and
X is a nucleoside or a nucleoside analog.

In another aspect, the invention relates to compounds of formula (B-II) and pharmaceutically acceptable salts thereof:

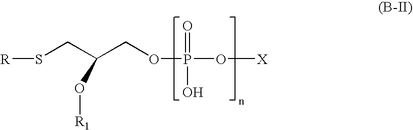

wherein
R is optionally substituted C1-C24, straight-chained or branched, unsaturated or saturated alkyl;
R₁ is optionally substituted C1-C24 straight-chained or branched, unsaturated or saturated alkyl;
n is 1, 2 or 3; and
X is a nucleoside or a nucleoside analog.

In another aspect, the invention relates to a composition comprising

i) an effective amount of a compound of formula (A-I) and pharmaceutically acceptable salts thereof:

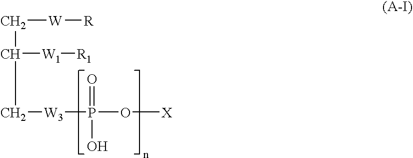

wherein
W is —S—, —S(O)—, —O—, —S—C═C—, —SC(O)—, —O—C═C— or —OC(O)—;
W₁ is —S—, —S(O)—, —O—, —S—C═C—, —SC(O)—, —O—C═C— or —OC(O)—;
W₃ is —O— or a covalent bond;
n is 1, 2 or 3;
R is C1-C7, C8-C12, C13-C18, or C19-C24 cyclic, straight-chained or branched, unsaturated or saturated alkyl which can be optionally substituted;
R₁ is C1, C2, C3-C10, C11-C15, C16-C17, or C18-C24 cyclic, straight-chained or branched, unsaturated or saturated alkyl which can be optionally substituted; and
X is a nucleoside or nucleoside analog radical; and
   ii) a pharmaceutically acceptable carrier.

In another aspect, the invention relates to a composition comprising

i) an effective amount of a compound of formula (A-II) and pharmaceutically acceptable salts thereof:

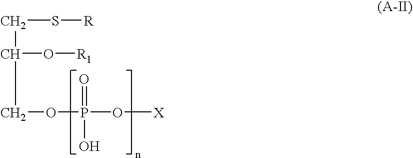

wherein
n is 1, 2 or 3;
R is C1-C7, C8-C12, C13-C18, or C19-C24 cyclic, straight-chained or branched, unsaturated or saturated alkyl which can be optionally substituted;
R₁ is C1, C2, C3-C10, C11-C15, C16-C17, or C18-C24 cyclic, straight-chained or branched, unsaturated or saturated alkyl which can be optionally substituted; and
X is a nucleoside or nucleoside analog radical; and

ii) a pharmaceutically acceptable carrier.

In another aspect, the invention relates to a composition comprising

i) an effective amount of a compound of formula (B-I) and pharmaceutically acceptable salts thereof:

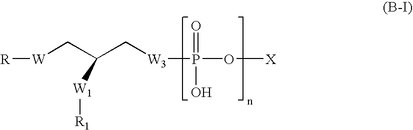

wherein
W is —S—, —S(O)—, —O—, —S—C═C—, —SC(O)—, —O—C═C— or —OC(O)—;
W₁ is —S—, —S(O)—, —O—, —S—C═C—, —SC(O)—, —O—C═C— or —OC(O)—;
W₃ is —O— or a covalent bond;
n is 1, 2 or 3;
R is optionally substituted, C1-C24, straight-chained or branched, unsaturated or saturated alkyl;
R₁ is optionally substituted C1-C24 straight-chained or branched, unsaturated or saturated alkyl; and
X is a nucleoside or a nucleoside analog; and

ii) a pharmaceutically acceptable carrier.

In another aspect, the invention relates to a composition comprising

i) an effective amount of a compound of formula (B-II) and pharmaceutically acceptable salts thereof:

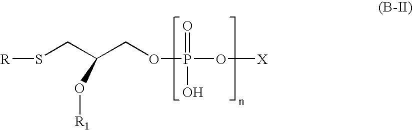

wherein
R is optionally substituted C1-C24, straight-chained or branched, unsaturated or saturated alkyl;
R₁ is optionally substituted C1-C24 straight-chained or branched, unsaturated or saturated alkyl;
n is 1, 2 or 3; and
X is a nucleoside or a nucleoside analog; and

ii) a pharmaceutically acceptable carrier.

In another aspect, the invention relates to a composition comprising

i) an effective amount of a first compound of structure (C-I) and pharmaceutically acceptable salts thereof:

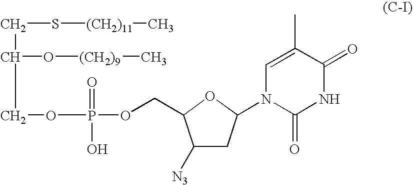

and

ii) an effective amount of a second compound of structure (C-II), (C-III), (C-IV), (C-V) or (C-VI):

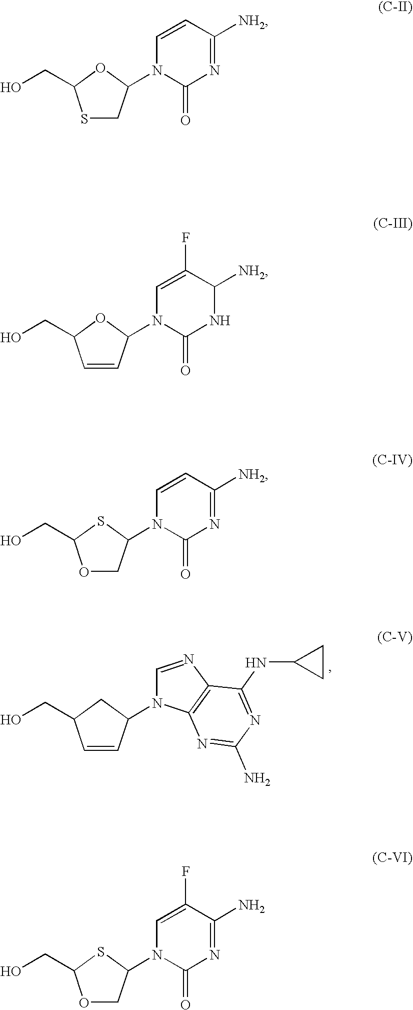

and pharmaceutically acceptable salts thereof.

In another aspect, the invention relates to a composition comprising

i) an effective amount of a first compound of formula (C-VII) and pharmaceutically acceptable salts thereof:

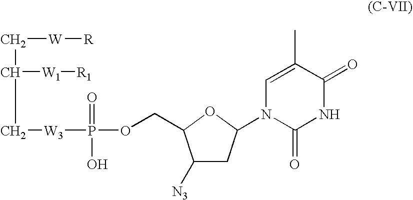

wherein
W is —S—, —S(O)—, —O—, —S—C═C—, —SC(O)—, —O—C═C— or —OC(O)—;
W₁ is —S—, —S(O)—, —O—, —S—C═C—, —SC(O)—, —O—C═C— or —OC(O)—;
W₃ is —O— or a covalent bond;
R is C1-C7, C8-C12 or C19-C24 cyclic, straight-chained or branched, unsaturated or saturated alkyl;
R₁ is C1, C2, C3-C10, C1-C15 or C18-C24 cyclic, straight-chained or branched, unsaturated or saturated alkyl; and

ii) an effective amount of a second compound of structure (C-II), (C-III), (C-IV), (C-V) or (C-VI) and pharmaceutically acceptable salts thereof:

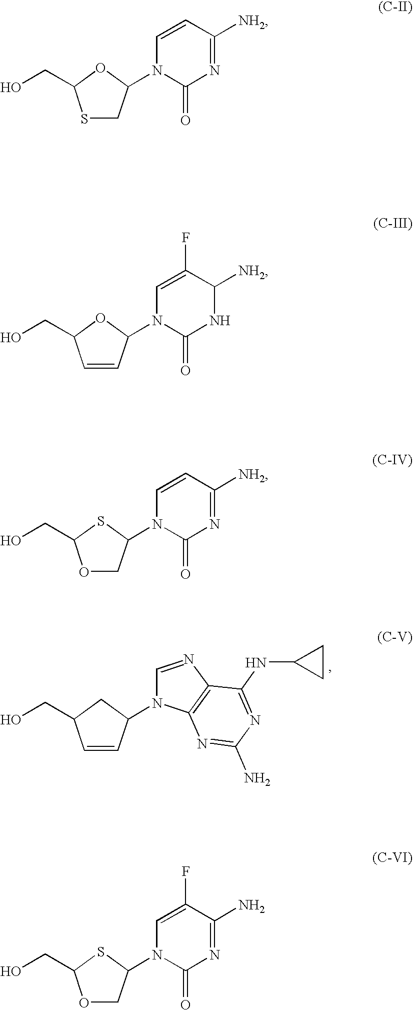

In another aspect, the invention relates to a method of inhibiting a reverse transcriptase enzyme comprising contacting the enzyme with an effective amount of a compound or pharmaceutically acceptable salt thereof of formula (A-I).

In another aspect, the invention relates to a method of inhibiting a reverse transcriptase enzyme comprising contacting the enzyme with an effective amount of a compound or pharmaceutically acceptable salt thereof of formula (A-II).

In another aspect, the invention relates to a method of inhibiting a reverse transcriptase enzyme comprising contacting the enzyme with an effective amount of a compound or pharmaceutically acceptable salt thereof of formula (B-I).

In another aspect, the invention relates to a method of inhibiting a reverse transcriptase enzyme comprising contacting the enzyme with an effective amount of a compound or pharmaceutically acceptable salt thereof of formula (B-II).

In another aspect, the invention relates to a method of inhibiting viral replication comprising contacting the virus with an effective amount of a compound or pharmaceutically acceptable salt thereof of formula (A-I).

In another aspect, the invention relates to a method of inhibiting viral replication comprising contacting the virus with an effective amount of a compound or pharmaceutically acceptable salt thereof of formula (A-II).

In another aspect, the invention relates to a method of inhibiting viral replication comprising contacting the virus with an effective amount of a compound or pharmaceutically acceptable salt thereof of formula (B-I).

In another aspect, the invention relates to a method of inhibiting viral replication comprising contacting the virus with an effective amount of a compound or pharmaceutically acceptable salt thereof of formula B-II).

In another aspect, the invention relates to a method for treating a viral disease, comprising administering to a subject in need of thereof an effective amount of a compound of formula (A-I) or a pharmaceutically acceptable salt thereof.

In another aspect, the invention relates to a method for treating a viral disease, comprising administering to a subject in need of thereof an effective amount of a compound of formula (A-II) or a pharmaceutically acceptable salt thereof.

In another aspect, the invention relates to a method for treating a viral disease, comprising administering to a subject in need of thereof an effective amount of a compound of formula (B-I) or a pharmaceutically acceptable salt thereof.

In another aspect, the invention relates to a method for treating a viral disease, comprising administering to a subject in need of thereof an effective amount of a compound of formula (B-II) or a pharmaceutically acceptable salt thereof.

In another aspect, the invention relates to a method for treating cancer, comprising administering to a subject in need of thereof an effective amount of a compound of formula (A-I), (A-II), (B-I) or (B-II) or a pharmaceutically acceptable salt thereof.

In another aspect, the invention relates to a method for treating a viral disease, comprising administering to a subject in need of thereof:

i) an effective amount of a first compound of structure (C-I):

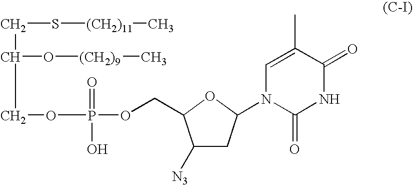

or a pharmaceutically acceptable salt thereof; and

ii) an effective amount of a second compound of structure (C-II), (C-III), (C-IV), (C-V) or (C-VI) or a pharmaceutically acceptable salt thereof:

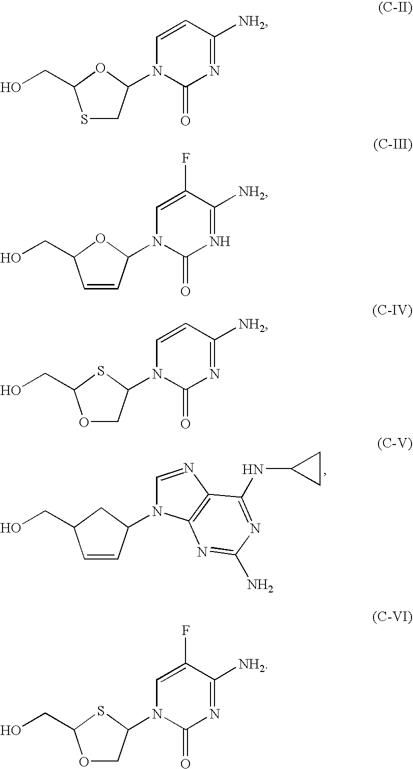

In another aspect, the invention relates to a method for treating a viral disease, comprising administering to a subject in need of thereof:

i) an effective amount of a first compound of formula (C-VII) or a pharmaceutically acceptable salt thereof:

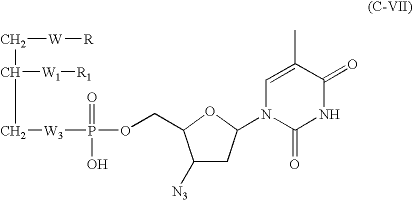

wherein

W is —S—, —S(O)—, —O—, —S—C═C—, —SC(O)—, —O—C═C— or —OC(O)—;

W₁ is —S—, —S(O)—, —O—, —S—C═C—, —SC(O)—, —O—C═C— or —OC(O)—;

W₃ is —O— or a covalent bond;

R is C1-C7, C8-C12 or C19-C24 cyclic, straight-chained or branched, unsaturated or saturated alkyl;

R₁ is C1, C2, C3-C10, C11-C15 or C18-C24 cyclic, straight-chained or branched, unsaturated or saturated alkyl; and

ii) an effective amount of a second compound of structure (C-II), (C-III), (C-IV), (C-V) or (C-VI) or a pharmaceutically acceptable salt thereof:

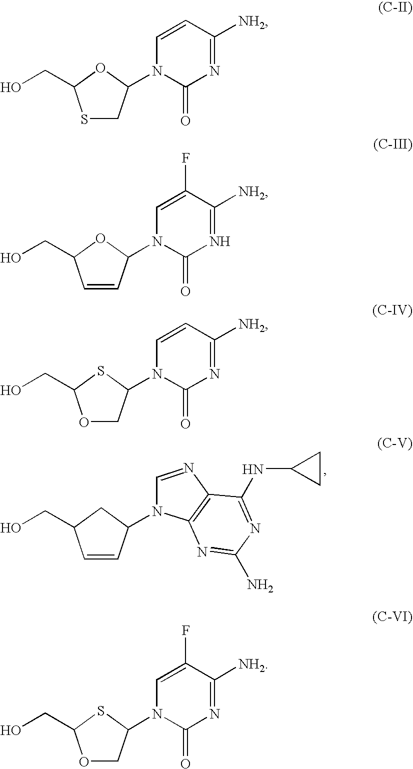

In another aspect, the invention relates to a method for treating cancer, comprising administering to a subject in need of thereof

i) an effective amount of a first compound of structure (C-I) or a pharmaceutically acceptable salt thereof; and

ii) an effective amount of a second compound of structure (C-II), (C-III), (C-IV), (C-V) or (C-VI) or a pharmaceutically acceptable salt thereof.

In another aspect, the invention relates to a method for treating cancer, comprising administering to a subject in need of thereof

i) an effective amount of a first compound of structure (C-VII) or a pharmaceutically acceptable salt thereof; and

ii) an effective amount of a second compound of structure (C-II), (C-III), (C-IV), (C-V) or (C-VI) or a pharmaceutically acceptable salt thereof.

In another aspect, the invention relates to a kit comprising a compound of formula (A-I) or a pharmaceutically acceptable salt thereof contained in a container and instructions for use.

In another aspect, the invention relates to a kit comprising a compound of formula (A-II) or a pharmaceutically acceptable salt thereof contained in a container and instructions for use.

In another aspect, the invention relates to a kit comprising a compound of formula (B-I) or a pharmaceutically acceptable salt thereof contained in a container and instructions for use.

In another aspect, the invention relates to a kit comprising a compound of formula (B-II) or a pharmaceutically acceptable salt thereof contained in a container and instructions for use.

### DETAILED DESCRIPTION OF THE INVENTION

Reference will now be made in detail to various embodiments of the invention. Examples of the particular embodiments are illustrated in the following Examples section.

Unless defined otherwise, all technical and scientific terms used herein have the same meaning as is commonly understood by one of skill in the art to which this invention belongs. All patents and publications referred to herein are incorporated by reference.

### Certain Chemical Terminology

As used herein, C1-Cx includes C1-C2, C1-C3 . . . C1-Cx. By way of example only, a group designated as “C1-C4” indicates that there are one to four carbon atoms in the moiety, i.e. groups containing 1 carbon atom, 2 carbon atoms, 3 carbon atoms or 4 carbon atoms, as well as the ranges C1-C2 and C1-C3. Thus, by way of example only, “C1-C4 alkyl” indicates that there are one to four carbon atoms in the alkyl group. Whenever it appears herein, a numerical range such as “1 to 10” refers to each integer in the given range; e.g., “1 to 10 carbon atoms” means that the group may have 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

The term “alkyl” refers to a cyclic, straight-chained or branched, unsaturated or saturated hydrocarbon chain. The term “lower alkyl” refers to a cyclic, straight-chained or branched, unsaturated or saturated alkyl of one to ten carbon atoms. Unless otherwise indicated, alkyl means unsubstituted alky.

As used herein the term “halogen” refers to fluorine, bromine, chlorine, or iodine.

The term “alkoxy” refers to —OR′, wherein R′ is lower alkyl, substituted lower alkyl, acyl, aryl, substituted aryl, arylalkyl, substituted arylalkyl, heteroarylalkyl, cycloalkyl, substituted cycloalkyl, cycloheteroaryl or substituted cycloheteroaryl.

The term “carbonyl” refers to a carbon atom to which three atoms are covalently bound, wherein one of the three atoms is bound to the carbon atom via a double bond.

The term “alkylmercapto” refers to a sulfide group that is substituted with a lower alkyl group.

The term “alkylcarbonyl” refers to a carbonyl group that is substituted with a lower alkyl group.

The term “alkoxycarbonyl” refers to a carbonyl group that is substituted with an alkoxy group.

The term “alkylsulphonyl” refers to a sulphonyl group (—S(O)₂—) that is substituted with a lower alkyl group.

The term “aryl” refers to an unsaturated aromatic carbocyclic group of from 6 to 20 carbon atoms having a single ring (e.g., phenyl) or multiple condensed (fused) rings (e.g., naphthyl or anthryl).

The term “substituted alkyl” refers to an alkyl group as defined above, having from 1 to 5 substituents, including, but not limited to alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl; or an alkyl group as defined above that is interrupted by 1-20 atoms independently chosen from oxygen, sulfur and NR^{a}—, where R^{a} is chosen from hydrogen, or optionally substituted alkyl, cycloalkyl, alkenyl, cycloalkenyl, alkenyl, cycloalkenyl, alkynyl, aryl, heteroaryl and heterocyclic; or an alkyl group as defined above that has both from 1 to 5 substituents as defined above and is also interrupted by 1-20 atoms as defined above.

### Certain Pharmaceutical Terminology

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The compounds described herein may possess acidic or basic groups and therefore may react with any of a number of inorganic or organic bases, and inorganic and organic acids, to form a pharmaceutically acceptable salt. These salts can be prepared in situ during the final isolation and purification of the compounds of the invention, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. Other acids, such as oxalic, while not in themselves pharmaceutically acceptable, may be employed in the preparation of salts useful as intermediates in obtaining the compounds of the invention and their pharmaceutically acceptable acid addition salts.

Those compounds described herein which may comprise a free acid group may react with a suitable base, such as the hydroxide, carbonate, bicarbonate, sulfate, of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, N⁺ (C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they may contain. Water or oil-soluble or dispersible products may be obtained by such quaternization. The compounds described herein can be prepared as pharmaceutically acceptable salts formed when an acidic proton present in the parent compound either is replaced by a metal ion, for example an alkali metal ion, an alkaline earth ion, or an aluminum ion; or coordinates with an organic base. Base addition salts can also be prepared by reacting the free acid form of the compounds described herein with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like. In addition, the salt forms of the disclosed compounds can be prepared using salts of the starting materials or intermediates.

The term “effective amount” refers to that amount of a compound or composition of the invention that is effective for treating a viral disease or cancer, when administered to a subject in need of such treatment. The therapeutically effective amount may vary depending upon the subject and disease condition being treated, the weight and age of the subject, the severity of the disease condition, the manner of administration and the like, which can readily be determined by one of ordinary skill in the art. The specific dose may vary depending on the particular compound chosen, the dosing regimen to be followed, timing of administration, and the physical delivery system in which it is carried. When a compound of the invention and another agent are administered for treating a viral disease or cancer, the effective amount is the total amount of the compound of the invention and the other agent that is useful for treating a viral disease or cancer.

The term “treating” and its grammatical equivalents as used herein include achieving a therapeutic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

The terms “prevent” or “preventing” refer to administration of a compound or composition described herein to a patient at risk of developing a particular disease, or to a patient reporting one or more of the physiological symptoms of a disease, even though a diagnosis of this disease may not have been made.

The term “subject” as used herein in reference to an individual suffering from a disorder, encompasses mammals and non-mammals. Mammals are any member of the Mammalian class, including but not limited to humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds,

fish and the like. In some embodiments of the methods and compositions provided herein, the subject is a mammal. In some embodiments, the subject is a human.

The terms “co-administration”, “administered in combination with” and their grammatical equivalents, as used herein, encompass administration of two or more agents to a subject so that both agents and/or their metabolites exhibit a therapeutic effect concurrently. Co-administration includes simultaneous administration in separate compositions, administration at different times in separate compositions, or administration in a composition in which both agents are present.

### Compounds

In one aspect the present invention is directed to compounds of formula (A-I) below and pharmaceutically acceptable salts thereof:

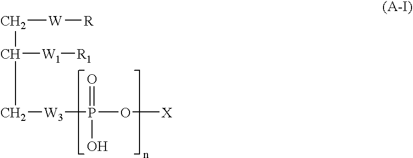

wherein

### W is —S, —SO, —O, —S—C═C, —SC(O), —O—C═C, —OC(O);

### W₁ is —S, —SO, —O, —S—C═C, —SC(O), —O—C═C, —OC(O);

W₃ is —O— or a covalent bond;
n is 1, 2 or 3;
R is C1-C7, C8-C12, C13-C18, or C19-C24 cyclic, straight-chained or branched, unsaturated or saturated alkyl which can be optionally substituted;
R₁ is C1, C2, C3-C10, C11-C15, C16-C17, or C18-C24 cyclic, straight-chained or branched, unsaturated or saturated alkyl which can be optionally substituted; and X is a nucleoside or nucleoside analog radical.

In some embodiments W is —S or —SO. In another embodiment W is —O. In some embodiments, n is 1.

In some embodiments, R and R₁ are independently substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonyl groups.

In some embodiments R is a C8-C12 cyclic, straight-chained or branched, unsaturated or saturated alkyl which can be optionally substituted. In some embodiments R is a C12 straight-chained or branched, unsaturated or saturated alkyl which can be optionally substituted.

In some embodiments, R₁ is a C1, C2, or C3-C10 cyclic, straight-chained or branched, unsaturated or saturated alkyl which can be optionally substituted. In some embodiments R₁ is a C2 unsaturated or saturated alkyl which can be optionally substituted.

In some embodiments, X is cytidine, a cytidine analog, uridine, a uridine analog, adenosine, an adenosine analog, guanosine, a guanosine analog, thymidine, a thymidine analog, inosine or an inosine analog radical.

In one embodiment, X is cytidine or a cytidine analog radical. Examples of cytidine analogs include, but are not limited to, deoxycytidine; 2′,3′-dideoxycytidine; 2′,3′-didehydrocytidine carbocyclic; 2′,3′-didehydro-2′,3′-dideoxycytidine (D4C); 2′,3′-didehydro-2′,3′-dideoxy-5-methylcytidine (D4MeC); fluoro-2′,3′-dideoxycytidine (5-F-ddC); 3-(4-hydroxy-1′,2′-butadienyl)cytosine; 3′-azido-2′,3′-dideoxy-5-methylcytosine (AzddMeC); 3′-azido-2′,3′-dideoxy-5-methylcytosine (AzddMeC); 3′-azido-2′,3′-dideoxy-5-methylcytosine-N4OH (AzddMeC N4-OH); 3′-azido-2′,3′-dideoxy-5-methylcytosine-N4Me, (AzddMeC N4Me); 3′-azido-2′,3′-dideoxycytosine (AzddC); 3′-azido-2′,3′-dideoxy-5-fluorocytosine (AzddFC); 2′,3′-dideoxy-2′,3′-didehydrocytidine; and beta-L-5-fluoro-2′,3′-dideoxy-2′,3′-didehydrocytidine. In another embodiment, X is cytidine.

In one embodiment, X is uridine or a uridine analog radical. Examples of uridine analogs include, but are not limited to, deoxyuridine; 5-Methyluridine; 3′-azido-2′,3′-dideoxy-5-chlorouridine (AzddClU); 3′-azido-2′,3′-dideoxy-5-ethyluridine (AzddEtU); 3′-azido-2′,3′-dideoxyuridine (AzddU); 3′-fluoro-2′,3′-dideoxy-5-bromouridine (3′FddBrU); 3′-fluoro-2′,3′-dideoxy-5-ethyluridine (3′FddEtU); 3′-azido-2′,3′-dideoxy-5-bromouridine (AzddBrU); 3′-azido-2′,3′-dideoxyuridine (AzddIU); 3′-fluoro-2′,3′-dideoxy-5-chlorouridine (FddClU); 3′-fluoro-2′,3′-dideoxyuridine (3′FddU); 2′,3′-dideoxy-3′-azidouridine; and 2′,3′-dideoxy-3′-3′-fluoro-5-chlorouridine. In another embodiment, X is uridine.

In another embodiment X is adenosine or an adenosine analog radical. Example of adenosine analogs include, but are not limited to, deoxyadenosine; 2′,3′-dideoxyadenosine; 2′,3′-dideoxy-2′-fluoro-ara-adenosine; 2-chlorodeoxyadenosine; 9-(4-hydroxy-1′,2′-butadienyl)adenine; 9-(2-phosphonomethoxyethyl)adenine; 2′,3′-didehydro-2′,3′-dideoxyadenosine (D4A); dideoxyadenosine (ddA); 5-methyl-2′,3′-dideoxyadenosine (ddMeA); 3′-fluoro-2′,3′-dideoxy-arabinofuranosyl-adenine (3-Fddara-A); 3′-fluoro-2′,3′-dideoxyadenosine (3-FddA); 2′,3′-dideoxy-2′,3′-didehydro-N-6-(O-methylbenzyl)adenosine; 2′,3′-dideoxy-2′,3′-didehydro-N-6-(2-methylpropyl)adenosine; and 2′,3′-dideoxy-3′-fluoroadenosine. In another embodiment, X is adenosine.

In another embodiment, X is guanosine or a guanosine analog radical. Examples of guanosine analogs include, but are not limited to, of deoxyguanosine; 2′,3′-dideoxyguanosine; 2′,3′-didehydroguanosine; 3′-azido-3′-deoxyguanosine; 3′-fluoro-2′,3′-dideoxyguanosine; dideoxyguanosine (ddG); 3′-azideo-2′,3′-dideoxyguanosine (3-N.sub.3 ddG); 3′-fluoro-2′,3′-dideoxyguanosine (3-FddG); and 2′,3′-dideoxy-3′-azidoguanosine. In another embodiment, X is guanosine.

In yet another embodiment, X is thymidine or a thymidine analog radical. Examples of thymidine analogs include, but are not limited to, deoxythymidine; 3′-deoxythymidine; 2′,3′-dideoxythymidine; 2′,3′-didehydrothymidine; 3′-azido-3′-deoxythymidine; 3′-fluoro-3′-deoxythymidine; 3′-fluoro-2′,3′-dideoxythymidine (3′FddT); 3′-deoxy-2′,3′-didehydrothymidine; and 2′,3′-didehydro-2′,3′-dideoxythymidine (D4T). In another embodiment, X is thymidine.

In yet another embodiment, X is inosine or an inosine analog radical. Examples of inosine analogs include, but are not limited to, deoxyinosine; dideoxyinosine (ddI); and 2′,3′-dideoxyinosine. In another embodiment, X is inosine.

In some embodiments X is a radical of 2,6-diaminopurine-2′,3′-dideoxyriboside; 2,6-diaminopurine-3′-azido-2′,3′-dideoxyriboside; 2,6-diaminopurine-3′-fluoro-2′,3′-dideoxyriboside; 3-phosphonomethoxyethyl-2,6-diaminopurine; 2,6-diaminopurine-2′,3′-dideoxyriboside (ddDAPR); 3′-azido-2′,3′-dideoxy-diaminopurine (N₃ ddDAPR); 3′-fluoro-2′,3′-dideoxy-diaminopurine (3-FddDAPR); or 2′,3′-dideoxy-3′-fluoro-2,6-diaminopurineriboside.

In other embodiments X is a radical of Abacavir, Aciclovir, Adefovir, Alovudine, Amantadine, amprenavir, Cidofovir, Cytarabine, Desciclovir, Didanosine, Docosanol, Edoxudine, Elvucitabine, Emtricitabine, Famciclovir, Fomivirsen, Foscarnet, Ganciclovir, Idoxuridine, Lamivudine, Oseltamivir, Penciclovir, Peramivir, Rimantadine, Ribavirin, Stavudine, Tenofovir, Tenofovir, Fiacitabine, Fialuridine, doxuridine, Foscamet, Lobucavir, Sorivudine, Trifluridine, Tromantadine, ribavirine, stavudine, Valaciclovir, Valganciclovir, Vidarabine, Viramidine, Zalcitabine, Zanamivir, or Zidovudine. In some embodiments, X is alovudine, ribavirin, Zidovudine, Viramidine and Elvucitabine.

The invention also provides compounds of formula (A-II) below and pharmaceutically acceptable salts thereof:

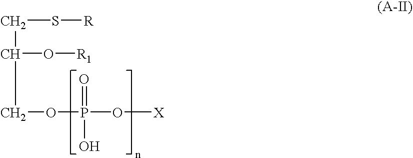

wherein R, R₁, n and X are defined as described in formula (A-I) above.

In addition, provided herein are compounds of formula (A-III) below and pharmaceutically acceptable salts thereof:

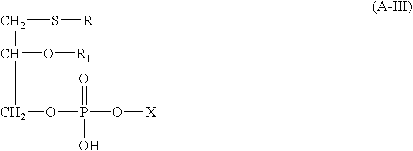

wherein R, R₁ and X are defined and described in formula (A-I) above.

Further, the invention also provides compounds of formula (A-IV) below and pharmaceutically acceptable salts thereof:

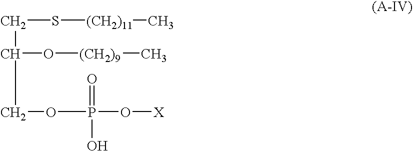

wherein X is defined and described in formula (A-I) above.

Additionally, provided herein is a compounds of formula (A-V) below and pharmaceutically acceptable salts thereof:

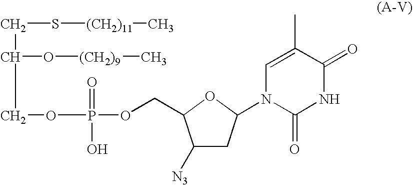

The compounds of general formulae (A-I) to (A-V) contain one or more asymmetrical carbon atoms. Unless otherwise indicated, all optically-active forms, including enantiomers, diastereomers, racemates and racemic mixtures thereof of the compounds are also the subject of the present invention.

In another aspect the invention provides methods for inhibiting a reverse transcriptase enzyme comprising contacting the enzyme with an effective amount of a compound or pharmaceutically acceptable salt thereof of formulas (A-I)-(A-XIV). In some embodiments, the contacting is in a cell. In some embodiments, the contacting is in an organ. In some embodiments, the contacting is in a tissue. In some embodiments, the contacting is in an organism. In some embodiments, the contacting takes place extracellularly.

In another aspect the invention provides methods for inhibiting viral replication comprising contacting the virus with an effective amount of a compound or pharmaceutically acceptable salt thereof of formulas (A-I)-(A-XIV). In some embodiments, the viral replication occurs in a cell. In some embodiments, the viral replication occurs in an organ. In some embodiments, the viral replication occurs in a tissue. In some embodiments the viral replication occurs in an organism. In some embodiments, the viral replication occurs in extracellularly. In some embodiments, the virus is herpes simplex virus, cytomegalovirus, Papovavirus, varicella zoster virus, or Epstein-Barr virus, togaviruses, retroviruses, HTLV-I, HTLV-II, lentiviruses, HIV-1 or HIV-2. In some embodiments, the virus is HIV-1 or HIV-2.

In another aspect the invention is directed to methods of treating subject in need thereof by administering an effective amount of the compounds or pharmaceutically acceptable salts thereof of formula (A-I)-(A-XIV) to the subject. In one embodiment the disorder is a viral disease.

Also, the invention provides methods of treating a subject in need thereof comprising administering an effective amount of a compound or pharmaceutically acceptable salts thereof of formula (A-VI):

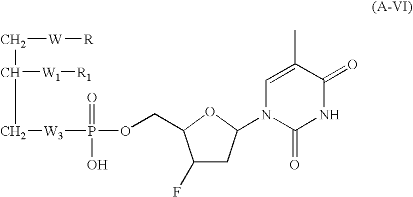

wherein W, W₁, W₃, R, and R₁ are defined and described in formula (A-I) above. In one embodiment the disorder is a viral disease.

In another aspect, the invention provides pharmaceutical compositions comprising the compounds or pharmaceutically acceptable salts thereof of formulas (A-I)-(A-XIV) and a pharmaceutically acceptable carrier.

In yet another aspect, the invention provides kits comprising the compounds or pharmaceutically acceptable salts thereof of formula (A-I) contained in a container and instructions for use.

The compounds of formula (A-I) are moieties having a phospholipid group represented by a short alkyl backbone (CH₂—CH—CH₂ in formula (A-I)), a hydrophobic end (R and R₁) linked to one end of the alkyl backbone by the functional group W and W₁, and a phosphate group (or multiple phosphate groups) linked to the alkyl backbone by functional group W₃. Without being limited to any theory, the lipophilicity of R and R₁ may allow the compounds of formula (A-I) to interact with the cell membrane, e.g., bind with the cell membrane of cells to provide an anchor thereto or to cross the cell membrane.

In one aspect of the invention, these phospholipid groups may avoid or reduce the side effects associated with many nucleoside analogs. A nucleoside or nucleoside analog radical (X in formula (A-I)), is attached via a phosphate group (or multiple phosphate group) to a lipid. The nucleoside/nucleoside analog-phosphate-lipid can be cleaved by an enzyme that is present in some, but not all, cells to the nucleoside/nucleoside analog-(mono-, di-, or tri-) phosphate. The nucleoside/nucleoside analog-phosphate may be metabolized within the cells to the nucleoside/nucleoside analog-triphosphate, the active form.

In some embodiments W is —S; W₁ is —O; W₃ is —O; n is 1, 2 or 3; R is a C8-C12 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C9 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C10 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C11 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C12 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical.

In some embodiments, W is —S; W¹ is —O; W₃ is —O; n is 1, 2 or 3; R is a C1-C7 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C1 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C2 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C3 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C4 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C5 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C6 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C7 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical.

In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C14 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C15 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C16 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C17 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C18 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical.

In some embodiments, W is —S; W₁ is —O; W₃ is —O; n is 1, 2 or 3; R is a C19-C24 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C20 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is 40; W₃ is —O; n is 1, 2 or 3; R is a C21 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C22 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C23 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C24 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical. In some embodiments W is —S; W₁ is —; W₃ is —O; n is 1, 2 or 3; R is a C19-C24 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical.

In some embodiments W is —S; W₁ is 4; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical.

In some embodiments W is —S; W is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a CS cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a CS cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical. In some embodiments W is —S; W₁ is —O; W₃ is 40; n is 1, 2 or 3; R is a C19-C24 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical.

In some embodiments W is —S; W₁ is —O; W3 is —O; n is 1, 2 or 3; R is a C1-C7, C8-C12, C13-C18, or C19-C24 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical.

In some embodiments W is —S; W₁ is —O; W3 is —O; n is 1, 2 or 3; R is a C1-C7, C8-C12, C13-C18, or C19-C24 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical. In some embodiments W is —S; W₁ is —O; W3 is —O; n is 1, 2 or 3; R is a C1-C7 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C13 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C14 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C14 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C14 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C14 cyclic, straight-chained or branched, unsaturated or saturated alkyl which can be optionally substituted; and X is a nucleoside or nucleoside analog radical.

In some embodiments W is —S; W₁ is —O; W3 is —O; n is 1, 2 or 3; R is a C1-C7, C8-C12, C13-C18, or C19-C24 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C16 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C17 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C18 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C19 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C20 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C21 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C22 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C23 cyclic, straight-chained or branched, unsaturated or saturated alkyl which can be optionally substituted; and X is a nucleoside or nucleoside analog radical. In some embodiments W is —S; W₁ is —O; W3 is —O; n is 1, 2 or 3; R is a C1-C7, C8-C12, C13-C18, or C19-C24 cyclic, straight-chained or branched, unsaturated or saturated alkyl which can be optionally substituted; R₁ is a C24 cyclic, straight-chained or branched, unsaturated or saturated alkyl which can be optionally substituted; and X is a nucleoside or nucleoside analog radical.

In one embodiment, X is cytidine or a cytidine analog radical. Examples of cytidine analogs include, but are not limited to, deoxycytidine; 2′,3′-dideoxycytidine; 2′,3′-didehydrocytidine carbocyclic; 2′,3′-didehydro-2′,3′-dideoxycytidine (D4C); 2′,3′-didehydro-2′,3′-dideoxy-5-methylcytidine (D4MeC); fluoro-2′,3′-dideoxycytidine (5-F-ddC); 3-(4-hydroxy-1′,2′-butadienyl)cytosine; 3′-azido-2′,3′-dideoxy-5-methylcytosine (AzddMeC); 3′-azido-2′,3′-dideoxy-5-methylcytosine (AzddMeC); 3′-azido-2′,3′-dideoxy-5-methylcytosine-N4-OH (AzddMeC N4-OH); 3′-azido-2′,3′-dideoxy-5-methylcytosine-N4Me, (AzddMeC N4Me); 3′-azido-2′,3′-dideoxycytosine (AzddC); 3′-azido-2′,3′-dideoxy-5-fluorocytosine (AzddFC); 2′,3′-dideoxy-2′,3′-didehydrocytidine; and beta-L-5-fluoro-2′,3′-dideoxy-2′,3′-didehydrocytidine. In another embodiment, X is cytidine.

In another embodiment, X is uridine or a uridine analog radical. Examples of uridine analogs include, but are not limited to, deoxyuridine; 5-Methyluridine; 3′-azido-2′,3′-dideoxy-5-chlorouridine (AzddClU); 3′-azido-2′,3′-dideoxy-5-ethyluridine (AzddEtU); 3′-azido-2′,3′-dideoxyuridine (AzddU); 3′-fluoro-2′,3′-dideoxy-5-bromouridine (3′FddBrU); 3′-fluoro-2′,3′-dideoxy-5-ethyluridine (3′FddEtU); 3′-azido-2′,3′-dideoxy-5-bromouridine (AzddBrU); 3′-azido-2′,3′-dideoxyuridine (AzddIU); 3′-fluoro-2′,3′-dideoxy-5-chlorouridine (FddClU); 3′-fluoro-2′,3′-dideoxyuridine (3′FddU); 2′,3′-dideoxy-3′-azidouridine; and 2′,3′-dideoxy-3′-3′-fluoro-5-chlorouridine. In another embodiment, X is uridine.

In another embodiment, X is adenosine or an adenosine analog radical. Example of adenosine analogs include, but are not limited to, deoxyadenosine; 2′,3′-dideoxyadenosine; 2′,3′-dideoxy-2′-fluoro-ara-adenosine; 2-chlorodeoxyadenosine; 9-(4-hydroxy-1′,2′-butadienyl)adenine; 9-(2-phosphonomethoxyethyl)adenine; 2′,3′-didehydro-2′,3′-dideoxyadenosine (D4A); dideoxyadenosine (ddA); 5-methyl-2′,3′-dideoxyadenosine (ddMeA); 3′-fluoro-2′,3′-dideoxy-arabinofuranosyl-adenine (3-Fddara-A); 3′-fluoro-2′,3′-dideoxyadenosine (3-FddA); 2′,3′-dideoxy-2′,3′-didehydro-N6-(O-methylbenzyl)adenosine; 2′,3′-dideoxy-2′,3′-didehydro-N6-(2-methylpropyl)adenosine; and 2′,3′-dideoxy-3′-fluoroadenosine. In another embodiment, X is adenosine.

In another embodiment, X is guanosine or a guanosine analog radical. Examples of guanosine analogs include, but are not limited to, of deoxyguanosine; 2′,3′-dideoxyguanosine; 2′,3′-didehydroguanosine; 3′-azido-3′-deoxyguanosine; 3′-fluoro-2′,3′-dideoxyguanosine; dideoxyguanosine (ddG); 3′-azideo-2′,3′-dideoxyguanosine (3-N.sub.3 ddG); 3′-fluoro-2′,3′-dideoxyguanosine (3-FddG); and 2′,3′-dideoxy-3′-azidoguanosine. In another embodiment, X is guanosine.

In yet another embodiment, X is thymidine or a thymidine analog radical. Examples of thymidine analogs include, but are not limited to, deoxythymidine; 3′-deoxythymidine; 2′,3′-dideoxythymidine; 2′,3′-didehydrothymidine; 3′-azido-3′-deoxythymidine; 3′-fluoro-3′-deoxythymidine; 3′-fluoro-2′,3′-dideoxythymidine (3′FddT); 3′-deoxy-2′,3′-didehydrothymidine; and 2′,3′-didehydro-2′,3′-dideoxythymidine (D4T). In another embodiment, X is thymidine.

In yet another embodiment, X is inosine or an inosine analog radical. Examples of inosine analogs include, but are not limited to, deoxyinosine; dideoxyinosine (ddI); and 2′,3′-dideoxyinosine. In another embodiment, X is inosine.

In some embodiments X is a radical of 2,6-diaminopurine-2′,3′-dideoxyriboside; 2,6-diaminopurine-3′-azido-2′,3′-dideoxyriboside; 2,6-diaminopurine-3′-fluoro-2′,3′-dideoxyriboside; 3-phosphonomethoxyethyl-2,6-diaminopurine; 2,6-diaminopurine-2′,3′-dideoxyriboside (ddDAPR); 3′-azido-2′,3′-dideoxy-diaminopurine (N3 ddDAPR); 3′-fluoro-2′,3′-dideoxy-diaminopurine (3-FddDAPR); or 2′,3′-dideoxy-3′-fluoro-2,6-diaminopurineriboside.

In other embodiments X is a radical of Abacavir, Aciclovir, Adefovir, Alovudine, Amantadine, amprenavir, Cidofovir, Cytarabine, Desciclovir, Didanosine, Docosanol, Edoxudine, Elvucitabine, Emtricitabine, Famciclovir, Fomivirsen, Foscarnet, Ganciclovir, Idoxuridine, Lamivudine, Oseltamivir, Penciclovir, Peramivir, Rimantadine, Ribavirin, Stavudine, Tenofovir, Tenofovir, Fiacitabine, Fialuridine, doxuridine, Foscamet, Lobucavir, Sorivudine, Trifluridine, Tromantadine, ribavirine, stavudine, Valaciclovir, Valganciclovir, Vidarabine, Viramidine, Zalcitabine, Zanamivir, or Zidovudine. In some embodiments, X is alovudine, ribavirin, Zidovudine, Viramidine and Elvucitabine.

Compounds of formula (A-VI), (A-VII), (A-VIII), (A-IX) and (A-X) represent some embodiments of the compounds of formula (A-I).

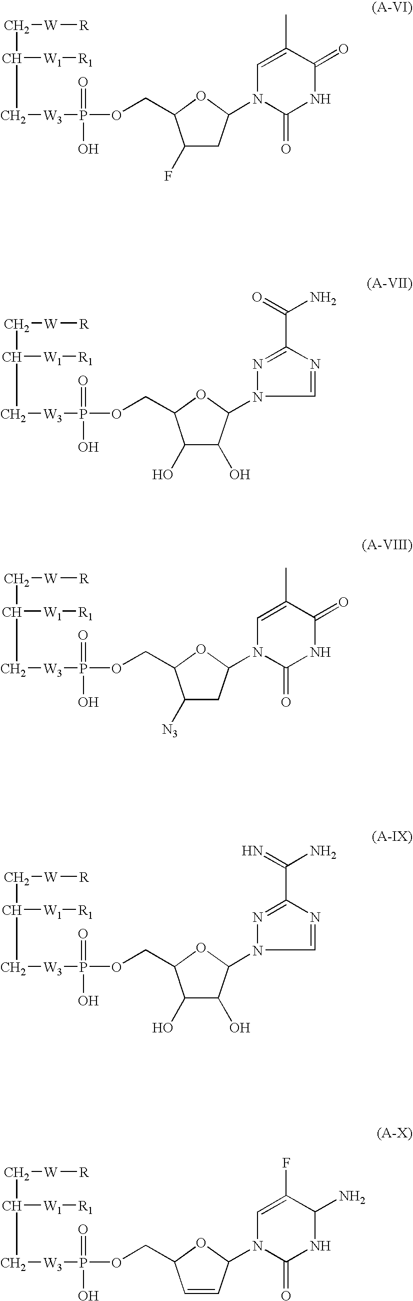

wherein W, W₁, W₃, R and R₁ are defined and described above in formula (A-I).

Compounds of formulae (A-II) and (A-III) represent some embodiments of the compounds of formula (A-I).

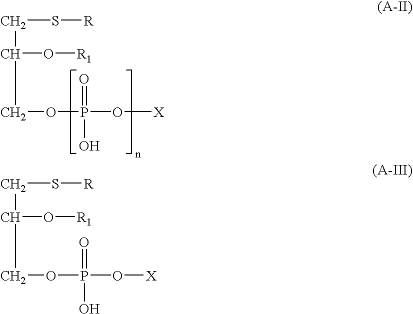

wherein R, R₁, n and X are defined and described above in formula (A-I)

Compounds of formulae (A-IV), (A-V), (A-XI), (A-XII), (A-XIII) and (A-XIV) represent some embodiments of the compounds of formula (A-III).

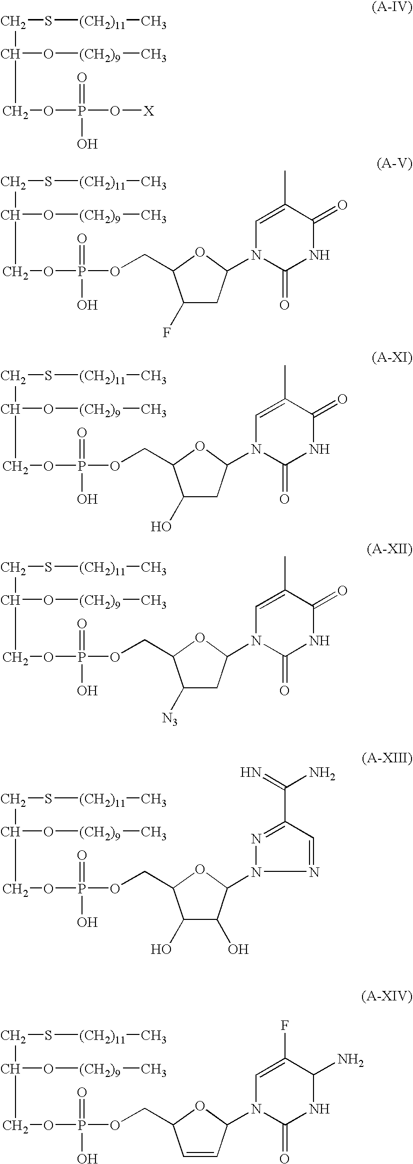

wherein X is defined and described above in formula (A-I).

In another aspect, the invention is directed to compounds of formula (B-I) and the pharmaceutically acceptable salts thereof:

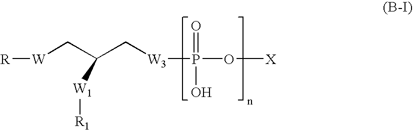

wherein

### W is —S, —SO, —O, —S—C═C, —SC(O), —O—C═C, —OC(O);

### W₁ is —S, —SO, —O, —S—C═C, —SC(O), —O—C═C, —OC(O);

W₃ is oxygen or a covalent bond;
n is 1, 2 or 3;
R is C1-C24 cyclic, straight-chained or branched, unsaturated or saturated alkyl which can be optionally substituted;
R₁ is C1-C24 cyclic, straight-chained or branched, unsaturated or saturated alkyl which can be optionally substituted; and
X is nucleoside or nucleoside analog radical.

In some embodiments W is —S or —SO. In another embodiment W is —O. In some embodiments, n is 1.

In some embodiments, R and R₁ are independently substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonyl groups.

In some embodiments R is a C8-C12 cyclic, straight-chained or branched, unsaturated or saturated alkyl which can be optionally substituted. In some embodiments R is a C12 straight-chained or branched, unsaturated or saturated alkyl which can be optionally substituted.

In some embodiments, R₁ is a C1, C2, or C3-C10 cyclic, straight-chained or branched, unsaturated or saturated alkyl which can be optionally substituted. In some embodiments R₁ is a C10 unsaturated or saturated alkyl which can be optionally substituted.

In some embodiments W is —S or —SO. In some embodiments W is —O. In another embodiment W₁ is —O. In some embodiments, n is 1. In some embodiments, R and R₁ are independently substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonyl groups.

In some embodiments R is C1-C7, C8-C12, C14, C13-C18, or C19-C24 cyclic, straight-chained or branched, unsaturated or saturated alkyl which can be optionally substituted. In some embodiments R is a C8-C12 cyclic, straight-chained or branched, unsaturated or saturated alkyl which can be optionally substituted. In some embodiments R is a C12 straight-chained or branched, unsaturated or saturated alkyl which can be optionally substituted.

In some embodiments R₁ is a C1, C2, C3-C10, C11-C15, C16-C17, or C18-C24 cyclic, straight-chained or branched, unsaturated or saturated alkyl which can be optionally substituted. In some embodiments, R₁ is a C1, C2, or C3-C10 cyclic, straight-chained or branched, unsaturated or saturated alkyl which can be optionally substituted. In some embodiments R₁ is a C10 unsaturated or saturated alkyl which can be optionally substituted.

The compounds of formula (B-I) are moieties having a phospholipid group represented by a short alkyl backbone (CH₂—CH—CH₂ in formula (B-I)), a hydrophobic end (R and R₁) linked to one end of the alkyl backbone by the functional group W and W₁, and a phosphate group (or multiple phosphate groups) linked to the alkyl backbone by functional group W₃. The lipophilicity of R and R₁ may allow the compounds of formula (B-I) to interact with the cell membrane, e.g., bind with the cell membrane of cells to provide an anchor thereto or to cross the cell membrane.

In one aspect of the invention, these phospholipid groups are designed to avoid or reduce the side effects associated with many nucleoside analogs. A nucleoside or nucleoside analog radical (X), is attached via a phosphate group (or multiple phosphate group) to a lipid. The nucleoside/nucleoside analog-phosphate-lipid may be cleaved by an enzyme that is present in some, but not all, cells to the nucleoside/nucleoside analog-(mono-, di-, or tri-) phosphate. The nucleoside/nucleoside analog-phosphate may be metabolized within the cells to the nucleoside/nucleoside analog-triphosphate, the active form. In another aspect of the invention, a specific enantiomer of the compounds of general formula (B-I) is less toxic than the other enantiomer.

In some embodiments W is —S or —SO. In another embodiment W is —O.

In some embodiments W₁ is —S or —SO. In another embodiment W₁ is —O.

In some embodiments, R and R₁ are independently substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonyl groups.

In some embodiments R is a C8-C12 cyclic, straight-chained or branched, unsaturated or saturated alkyl which can be optionally substituted. In some embodiments R is a C12 straight-chained or branched, unsaturated or saturated alkyl which can be optionally substituted.

In some embodiments, R₁ is a C1, C2, or C3-C10 cyclic, straight-chained or branched, unsaturated or saturated alkyl which can be optionally substituted. In some embodiments R₁ is a C2 unsaturated or saturated alkyl which can be optionally substituted.

In some embodiments, W is —S or —SO; W₁ is O; R is a C8-C12 cyclic, straight-chained or branched, unsaturated or saturated alkyl which can be optionally substituted and R₁ is a C1, C2, or C3-C10 cyclic, straight-chained or branched, unsaturated or saturated alkyl which can be optionally substituted. In some embodiments, W is —S or —SO; W₁ is O; R is a C12 straight-chained or branched, unsaturated or saturated alkyl which can be optionally substituted and R₁ is a C2 unsaturated or saturated alkyl which can be optionally substituted. In some embodiments, W is —S or —SO; W₁ is O; R is a C12 straight-chained or branched, unsaturated or saturated alkyl which can be optionally substituted and R₁ is a C10 unsaturated or saturated alkyl which can be optionally substituted.

In some embodiments, W is —S or —SO; W₁ is O; R is a C8-C12 cyclic, straight-chained or branched, unsaturated or saturated alkyl which can be optionally substituted and R₁ is C11-C15 cyclic, straight-chained or branched, unsaturated or saturated alkyl which can be optionally substituted. In some embodiments, W is —S or —SO; W₁ is O; R is a C8-C12 cyclic, straight-chained or branched, unsaturated or saturated alkyl which can be optionally substituted and R₁ is C18-C24 cyclic, straight-chained or branched, unsaturated or saturated alkyl which can be optionally substituted.

In some embodiments, W is —S or —SO; W₁ is O; R is a C1-C7 cyclic, straight-chained or branched, unsaturated or saturated alkyl which can be optionally substituted and R₁ is C1, C2, or C3-C10cyclic, straight-chained or branched, unsaturated or saturated alkyl which can be optionally substituted. In some embodiments, W is —S or —SO; W₁ is O; R is a C1-C7 cyclic, straight-chained or branched, unsaturated or saturated alkyl which can be optionally substituted and R₁ is C11-C15 cyclic, straight-chained or branched, unsaturated or saturated alkyl which can be optionally substituted. In some embodiments, W is —S or —SO; W₁ is O; R is a C1-C7 cyclic, straight-chained or branched, unsaturated or saturated alkyl which can be optionally substituted and R₁ is C18-C24 cyclic, straight-chained or branched, unsaturated or saturated alkyl which can be optionally substituted.

In some embodiments, W is —S or —SO; W₁ is O; R is a C19-C24 cyclic, straight-chained or branched, unsaturated or saturated alkyl which can be optionally substituted and R₁ is C1, C2, or C3-C10 cyclic, straight-chained or branched, unsaturated or saturated alkyl which can be optionally substituted. In some embodiments, W is —S or —SO; W₁ is O; R is a C19-C24 cyclic, straight-chained or branched, unsaturated or saturated alkyl which can be optionally substituted and R₁ is C11-C15 cyclic, straight-chained or branched, unsaturated or saturated alkyl which can be optionally substituted. In some embodiments, W is —S or —SO; W₁ is O; R is a C19-C24 cyclic, straight-chained or branched, unsaturated or saturated alkyl which can be optionally substituted and R₁ is C18-C24 cyclic, straight-chained or branched, unsaturated or saturated alkyl which can be optionally substituted.

In some embodiments W is —S; W₁ is —O; W₃ is —O; n is 1, 2 or 3; R is a C8-C12 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C9 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C10 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C11 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C12 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical.

In some embodiments, W is —S; W₁ is —O; W₃ is —O; n is 1, 2 or 3; R is a C1-C7 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a Cl cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C2 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C3 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C4 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C5 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C6 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C7 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical.

In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C14 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C15 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C16 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C17 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C18 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical.

In some embodiments, W is —S; W₁ is —O; W₃ is —O; n is 1, 2 or 3; R is a C19-C24 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C20 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C21 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C22 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C23 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C24 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical.

In some embodiments W is —S; W₁ is —O; W3 is —O; n is 1, 2 or 3; R is a C1-C7, C8-C12, C13-C18, or C19-C24 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical.

In some embodiments W is —S; W₁ is —O; W3 is —O; n is 1, 2 or 3; R is a C1-C7, C8-C12, C13-C18, or C19-C24 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical. In some embodiments W is —S; W₁ is —O; W3 is —O; n is 1, 2 or 3; R is a C1-C7 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C13 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C14 cyclic, straight-chained or branched, unsaturated or saturated alkyl which can be optionally substituted; and X is a nucleoside or nucleoside analog radical. In some embodiments, W is —S; W₁ is —O; W₃ is 40; n is 1, 2 or 3; R is a C8-C12 cyclic, straight-chained or branched, unsaturated or saturated alkyl which can be optionally substituted; R₁ is a C14 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C14 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C14 cyclic, straight-chained or branched, unsaturated or saturated alkyl which can be optionally substituted; and X is a nucleoside or nucleoside analog radical.

In some embodiments W is —S; W₁ is —O; W3 is —O; n is 1, 2 or 3; R is a C1-C7, C8-C12, C13-C18, or C19-C24 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C16 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C17 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C18 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C19 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C20 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C21 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C22 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C23 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C24 cyclic, straight-chained or branched, unsaturated or saturated alkyl which can be optionally substituted; and X is a nucleoside or nucleoside analog radical.

In some embodiments, X is cytidine, a cytidine analog, uridine, a uridine analog, adenosine, an adenosine analog, guanosine, a guanosine analog, thymidine, a thymidine analog, inosine or an inosine analog radical.

In one embodiment, X is cytidine or a cytidine analog radical. Examples of cytidine analogs include, but are not limited to, deoxycytidine; 2′,3′-dideoxycytidine; 2′,3′-didehydrocytidine carbocyclic; 2′,3′-didehydro-2′,3′-dideoxycytidine (D4C); 2′,3′-didehydro-2′,3′-dideoxy-5-methylcytidine (D4MeC); fluoro-2′,3′-dideoxycytidine (5-F-ddC); 3-(4-hydroxy-1′,2′-butadienyl)cytosine; 3′-azido-2′,3′-dideoxy-5-methylcytosine (AzddMeC); 3′-azido-2′,3′-dideoxy-5-methylcytosine (AzddMeC); 3′-azido-2′,3′-dideoxy-5-methylcytosine-N4OH (AzddMeC N4-OH); 3′-azido-2′,3′-dideoxy-5-methylcytosine-N4Me, (AzddMeC N4Me); 3′-azido-2′,3′-dideoxycytosine (AzddC); 3′-azido-2′,3′-dideoxy-5-fluorocytosine (AzddFC); 2′,3′-dideoxy-2′,3′-didehydrocytidine; and beta-L-5-fluoro-2′,3′-dideoxy-2′,3′-didehydrocytidine. In another embodiment, X is cytidine.

In one embodiment, X is uridine or a uridine analog radical. Examples of uridine analogs include, but are not limited to, deoxyuridine; 5-Methyluridine; 3′-azido-2′,3′-dideoxy-5-chlorouridine (AzddClU); 3′-azido-2′,3′-dideoxy-5-ethyluridine (AzddEtU); 3′-azido-2′,3′-dideoxyuridine (AzddU); 3′-fluoro-2′,3′-dideoxy-5-bromouridine (3′FddBrU); 3′-fluoro-2′,3′-dideoxy-5-ethyluridine (3′FddEtU); 3′-azido-2′,3′-dideoxy-5-bromouridine (AzddBrU); 3′-azido-2′,3′-dideoxyuridine (AzddIU); 3′-fluoro-2′,3′-dideoxy-5-chlorouridine (FddClU); 3′-fluoro-2′,3′-dideoxyuridine (3′FddU); 2′,3′-dideoxy-3′-azidouridine; and 2′,3′-dideoxy-3′-3′-fluoro-5-chlorouridine. In another embodiment, X is uridine.

In another embodiment X is adenosine or an adenosine analog radical. Example of adenosine analogs include, but are not limited to, deoxyadenosine; 2′,3′-dideoxyadenosine; 2′,3′-dideoxy-2′-fluoro-ara-adenosine; 2-chlorodeoxyadenosine; 9-(4-hydroxy-1′,2′-butadienyl)adenine; 9-(2-phosphonomethoxyethyl)adenine; 2′,3′-didehydro-2′,3′-dideoxyadenosine (D4A); dideoxyadenosine (ddA); 5-methyl-2′,3′-dideoxyadenosine (ddMeA); 3′-fluoro-2′,3′-dideoxy-arabinofuranosyl-adenine (3-Fddara-A); 3′-fluoro-2′,3′-dideoxyadenosine (3-FddA); 2′,3′-dideoxy-2′,3′-didehydro-N6-(O-methylbenzyl)adenosine; 2′,3′-dideoxy-2′,3′-didehydro-N6-(2-methylpropyl)adenosine; and 2′,3′-dideoxy-3′-fluoroadenosine. In another embodiment, X is adenosine.

In another embodiment, X is guanosine or a guanosine analog radical. Examples of guanosine analogs include, but are not limited to, of deoxyguanosine; 2′,3′-dideoxyguanosine; 2′,3′-didehydroguanosine; 3′-azido-3′-deoxyguanosine; 3′-fluoro-2′,3′-dideoxyguanosine; dideoxyguanosine (ddG); 3′-azideo-2′,3′-dideoxyguanosine (3-N.sub.3 ddG); 3′-fluoro-2′,3′-dideoxyguanosine (3-FddG); and 2′,3′-dideoxy-3′-azidoguanosine. In another embodiment, X is guanosine.

In yet another embodiment, X is thymidine or a thymidine analog radical. Examples of thymidine analogs include, but are not limited to, deoxythymidine; 3′-deoxythymidine; 2′,3′-dideoxythymidine; 2′,3′-didehydrothymidine; 3′-azido-3′-deoxythymidine; 3′-fluoro-3′-deoxythymidine; 3′-fluoro-2′,3′-dideoxythymidine (3′FddT); 3′-deoxy-2′,3′-didehydrothymidine; and 2′,3′-didehydro-2′,3′-dideoxythymidine (D4T). In another embodiment, X is thymidine.

In yet another embodiment, X is inosine or an inosine analog radical. Examples of inosine analogs include, but are not limited to, deoxyinosine; dideoxyinosine (ddI); and 2′,3′-dideoxyinosine. In another embodiment, X is inosine.

In some embodiments X is a radical of 2,6-diaminopurine-2′,3′-dideoxyriboside; 2,6-diaminopurine-3′-azido-2′,3′-dideoxyriboside; 2,6-diaminopurine-3′-fluoro-2′,3′-dideoxyriboside; 3-phosphonomethoxyethyl-2,6-diaminopurine; 2,6-diaminopurine-2′,3′-dideoxyriboside (ddDAPR); 3′-azido-2′,3′-dideoxy-diaminopurine (N3 ddDAPR); 3′-fluoro-2′,3′-dideoxy-diaminopurine (3-FddDAPR); or 2′,3′-dideoxy-3′-fluoro-2,6-diaminopurineriboside.

In other embodiments X is a radical of Abacavir, Aciclovir, Adefovir, Alovudine, Amantadine, amprenavir, Cidofovir, Cytarabine, Desciclovir, Didanosine, Docosanol, Edoxudine, Elvucitabine, Emtricitabine, Famciclovir, Fomivirsen, Foscarnet, Ganciclovir, Idoxuridine, Lamivudine, Oseltamivir, Penciclovir, Peramivir, Rimantadine, Ribavirin, Stavudine, Tenofovir, Tenofovir, Fiacitabine, Fialuridine, doxuridine, Foscamet, Lobucavir, Sorivudine, Trifluridine, Tromantadine, ribavirine, stavudine, Valaciclovir, Valganciclovir, Vidarabine, Viramidine, Zalcitabine, Zanamivir, or Zidovudine. In some embodiments, X is alovudine, ribavirin, Zidovudine, Viramidine or Elvucitabine.

Illustrative compounds of formula (B-I) include compounds of formula (B-II), (B-III), (B-IV), (B-V) and (B-VI) and pharmaceutically acceptable salts thereof:

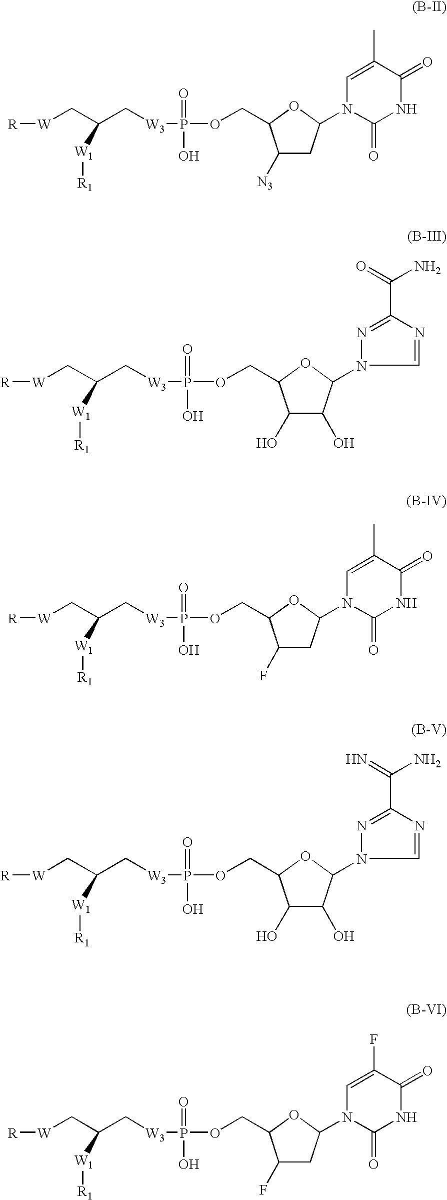

wherein W, W₁, W₃, R and R₁ are defined as described above in formula (B-I)

The invention also provides compounds of formula (B-VII), (B-VIII) and (B-IX) and pharmaceutically acceptable salts thereof, which represent some embodiments of the compounds of formula (B-I).

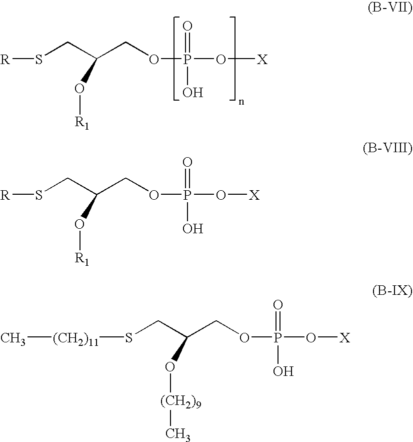

wherein R, R₁, n and X are defined as described in formula (B-I) above.

Additionally, provided herein are compounds of structure (B-X)-(B-XIV), and pharmaceutically acceptable salts thereof:

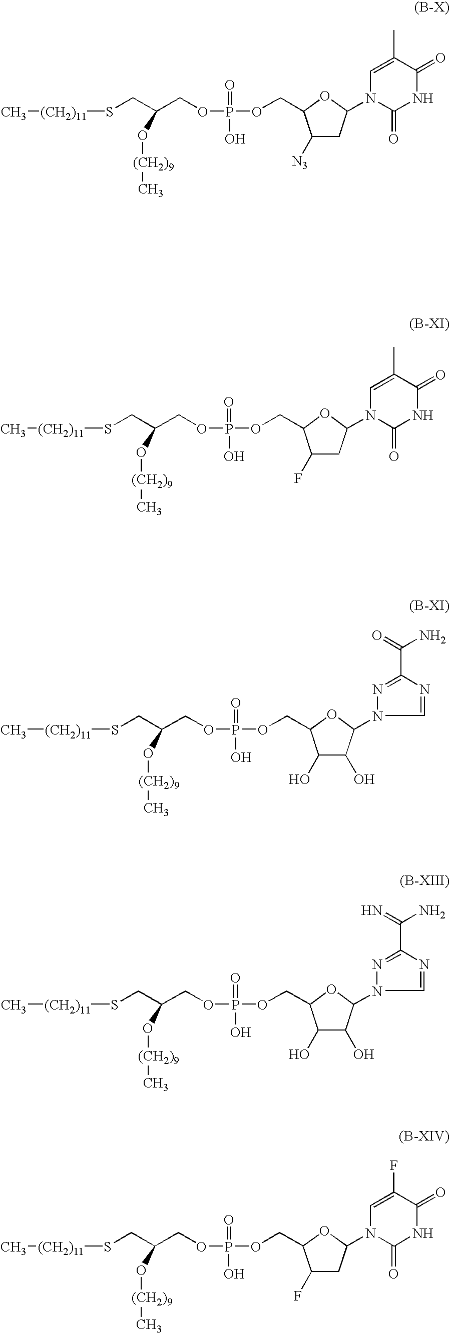

In other aspects, the invention provides compounds of formula (B-I-A) or pharmaceutically acceptable salts thereof.

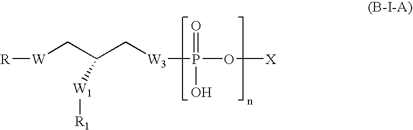

### W is —S, —SO, —O, —S—C═C, —SC(O), —O—C═C, —OC(O);

### W₁ is —S, —SO, —O, —S—C═C, —SC(O), —O—C═C, —OC(O);

W₃ is —O— or a covalent bond;
n is 1, 2 or 3;
R is C1-C24, straight-chained or branched, unsaturated or saturated alkyl which can be optionally substituted;
R₁ is C1-C24 straight-chained or branched, unsaturated or saturated alkyl which can be optionally substituted; and
X is a nucleoside or a nucleoside analog radical.

In some embodiments W is —S or —SO. In some embodiments W is —O. In another embodiment W₁ is —O. In some embodiments, n is 1. In some embodiments, R and R₁ are independently substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonylgroups.

In some embodiments R is C1-C7, C8-C12, C13-C18, or C19-C24 cyclic, straight-chained or branched, unsaturated or saturated alkyl which can be optionally substituted. In some embodiments R is a C8-C12 cyclic, straight-chained or branched, unsaturated or saturated alkyl which can be optionally substituted. In some embodiments R is a C12 straight-chained or branched, unsaturated or saturated alkyl which can be optionally substituted.

In some embodiments R₁ is a C1, C2, C3-C10, C11-C15, C16-C17, or C18-C24 cyclic, straight-chained or branched, unsaturated or saturated alkyl which can be optionally substituted. In some embodiments, R₁ is a C1, C2, or C3-C10 cyclic, straight-chained or branched, unsaturated or saturated alkyl which can be optionally substituted. In some embodiments R₁ is a C2 unsaturated or saturated alkyl which can be optionally substituted.

In one aspect of the invention, a specific enantiomer of the compounds of general formula (B-I) is less toxic than the other enantiomer.

In some embodiments W is —S or —SO. In another embodiment W is —O.

In some embodiments W₁ is —S or —SO. In another embodiment W₁ is —O.

In some embodiments, R and R₁ are independently substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonyl groups.

In some embodiments R is a C8-C12 cyclic, straight-chained or branched, unsaturated or saturated alkyl which can be optionally substituted. In some embodiments R is a C12 straight-chained or branched, unsaturated or saturated alkyl which can be optionally substituted.

In some embodiments, R₁ is a C1, C2, or C3-C10 cyclic, straight-chained or branched, unsaturated or saturated alkyl which can be optionally substituted. In some embodiments R₁ is a C2 unsaturated or saturated alkyl which can be optionally substituted.

In some embodiments, W is —S or —SO; W₁ is O; R is a C8-C12 cyclic, straight-chained or branched, unsaturated or saturated alkyl which can be optionally substituted and R₁ is a C1, C2, or C3-C10 cyclic, straight-chained or branched, unsaturated or saturated alkyl which can be optionally substituted. In some embodiments, W is —S or —SO; W₁ is O; R is a C12 straight-chained or branched, unsaturated or saturated alkyl which can be optionally substituted and R₁ is a C2 unsaturated or saturated alkyl which can be optionally substituted. In some embodiments, W is —S or —SO; W₁ is O; R is a C12 straight-chained or branched, unsaturated or saturated alkyl which can be optionally substituted and R₁ is a C10 unsaturated or saturated alkyl which can be optionally substituted.

In some embodiments, W is —S or —SO; W₁ is O; R is a C8-C12 cyclic, straight-chained or branched, unsaturated or saturated alkyl which can be optionally substituted and R₁ is C11-C15 cyclic, straight-chained or branched, unsaturated or saturated alkyl which can be optionally substituted. In some embodiments, W is —S or —SO; W₁ is O; R is a C8-C12 cyclic, straight-chained or branched, unsaturated or saturated alkyl which can be optionally substituted and R₁ is C18-C24 cyclic, straight-chained or branched, unsaturated or saturated alkyl which can be optionally substituted.

In some embodiments, W is —S or —SO; W₁ is O; R is a C1-C7 cyclic, straight-chained or branched, unsaturated or saturated alkyl which can be optionally substituted and R₁ is C1, C2, or C3-C₁₀ cyclic, straight-chained or branched, unsaturated or saturated alkyl which can be optionally substituted. In some embodiments, W is —S or —SO; W₁ is O; R is a C1-C7 cyclic, straight-chained or branched, unsaturated or saturated alkyl which can be optionally substituted and R₁ is C11-C15 cyclic, straight-chained or branched, unsaturated or saturated alkyl which can be optionally substituted. In some embodiments, W is —S or —SO; W₁ is O; R is a C1-C7 cyclic, straight-chained or branched, unsaturated or saturated alkyl which can be optionally substituted and R₁ is C18-C24 cyclic, straight-chained or branched, unsaturated or saturated alkyl which can be optionally substituted.

In some embodiments, W is —S or —SO; W₁ is O; R is a C19-C24 cyclic, straight-chained or branched, unsaturated or saturated alkyl which can be optionally substituted and R₁ is C1, C2, or C3-C10 cyclic, straight-chained or branched, unsaturated or saturated alkyl which can be optionally substituted. In some embodiments, W is —S or —SO; W₁ is O; R is a C19-C24 cyclic, straight-chained or branched, unsaturated or saturated alkyl which can be optionally substituted and R₁ is C11-C15 cyclic, straight-chained or branched, unsaturated or saturated alkyl which can be optionally substituted. In some embodiments, W is —S or —SO; W₁ is O; R is a C19-C24 cyclic, straight-chained or branched, unsaturated or saturated alkyl which can be optionally substituted and R₁ is C18-C24 cyclic, straight-chained or branched, unsaturated or saturated alkyl which can be optionally substituted.

In some embodiments W is —S; W₁ is —O; W₃ is —O; n is 1, 2 or 3; R is a C8-C12 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C9 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C10 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C11 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C12 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical.

In some embodiments, W is —S; W₁ is —O; W₃ is —O; n is 1, 2 or 3; R is a C1-C7 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a Cl cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C2 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C3 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C4 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C5 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C6 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C7 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical.

In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C14 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C15 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C16 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C17 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C18 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical.

In some embodiments, W is —S; W₁ is —O; W₃ is —O; n is 1, 2 or 3; R is a C19-C24 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C20 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C21 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is 40; n is 1, 2 or 3; R is a C22 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C23 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C24 cyclic, straight-chained or branched, unsaturated or saturated alkyl which can be optionally substituted; R₁ is a C1, C2, or C3-C10 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C1 cyclic, straight-chained or branched; unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C1 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C2 cyclic, straight-chained or branched, unsaturated or saturated alkyl which can be optionally substituted; and X is a nucleoside or nucleoside analog radical.

In some embodiments W is —S; W₁ is —O; W₃ is 4; n is 1, 2 or 3; R is a C1-C7, C8-C12, C13-C18, or C19-C24 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical. In some embodiments, W is S; W₁ is —O; W₃ is —O; n is 1, 2 or 3; R is a C13-C18 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C3 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C4 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C5 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C6 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C7 cyclic, straight-chained or branched, unsaturated or saturated alkyl which can be optionally substituted; and X is a nucleoside or nucleoside analog radical.

In some embodiments W is —S; W₁ is —O; W3 is —O; n is 1, 2 or 3; R is a C1-C7, C8-C12, C13-C18, or C19-C24 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C8 cyclic, straight-chained or branched, unsaturated or saturated alkyl which can be optionally substituted; and X is a nucleoside or nucleoside analog radical.

In some embodiments W is —S; W₁ is —O; W3 is —O; n is 1, 2 or 3; R is a C1-C7, C8-C12, C13-C18, or C19-C24 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical. In some embodiments, W is —S; W₁ is —O; W₃ is 40; n is 1, 2 or 3; R is a C8-C12 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C9 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical. In some embodiments W is —S; W₁ is —O; W3 is —O; n is 1, 2 or 3; R is a C1-C7 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C10 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C11 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C12 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C13 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7 cyclic, straight-chained or branched, unsaturated or saturated alkyl which can be optionally substituted; R₁ is a C14 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C14 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C14 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C14 cyclic, straight-chained or branched, unsaturated or saturated alkyl which can be optionally substituted; and X is a nucleoside or nucleoside analog radical.

In some embodiments W is —S; W₁ is —O; W3 is —O; n is 1, 2 or 3; R is a C1-C7, C8-C12, C13-C18, or C19-C24 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical. In some embodiments W is —S; W₁ is —O; W₃ is 40; n is 1, 2 or 3; R is a C1-C7 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C8-C12 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C13-C18 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C19-C24 cyclic, straight-chained or branched, unsaturated or saturated alkyl which can be optionally substituted; R₁ is a C15 cyclic, straight-chained or branched, unsaturated or saturated alkyl which can be optionally substituted; and X is a nucleoside or nucleoside analog radical.

In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C16 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C17 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C18 cyclic, straight-chained or branched, unsaturated or saturated alkyl which can be optionally substituted; and X is a nucleoside or nucleoside analog radical. In some embodiments, W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C19 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C20 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C21 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C22 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12; C13-C18, or C19-C24 cyclic, straight-chained or branched, unsaturated or saturated alkyl which can be optionally substituted; R₁ is a C23 cyclic, straight-chained or branched, unsaturated or saturated alkyl which can be optionally substituted; and X is a nucleoside or nucleoside analog radical. In some embodiments W is —S; W₁ is —O; W₃ is —O; n is 1, 2 or 3; R is a C1-C7, C8-C12, C13-C18, or C19-C24 cyclic, straight-chained or branched, unsaturated or saturated alkyl which can be optionally substituted; R₁ is a C24 cyclic, straight-chained or branched, unsaturated or saturated alkyl which can be optionally substituted; and X is a nucleoside or nucleoside analog radical.

In some embodiments, X is cytidine, a cytidine analog, uridine, a uridine analog, adenosine, an adenosine analog, guanosine, a guanosine analog, thymidine, a thymidine analog, inosine or an inosine analog radical.

In one embodiment, X is cytidine or a cytidine analog radical. Examples of cytidine analogs include, but are not limited to, deoxycytidine; 2′,3′-dideoxycytidine; 2′,3′-didehydrocytidine carbocyclic; 2′,3′-didehydro-2′,3′-dideoxycytidine (D4C); 2′,3′-didehydro-2′,3′-dideoxy-5-methylcytidine (D4MeC); fluoro-2′,3′-dideoxycytidine (5-F-ddC); 3-(4-hydroxy-1′,2′-butadienyl)cytosine; 3′-azido-2′,3′-dideoxy-5-methylcytosine (AzddMeC); 3′-azido-2′,3′-dideoxy-5-methylcytosine (AzddMeC); 3′-azido-2′,3′-dideoxy-5-methylcytosine-N4OH (AzddMeC N4-OH); 3′-azido-2′,3′-dideoxy-5-methylcytosine-N4Me, (AzddMeC N4Me); 3′-azido-2′,3′-dideoxycytosine (AzddC); 3′-azido-2′,3′-dideoxy-5-fluorocytosine (AzddFC); 2′,3′-dideoxy-2′,3′-didehydrocytidine; and beta-L-5-fluoro-2′,3′-dideoxy-2′,3′-didehydrocytidine. In another embodiment, X is cytidine.

In another embodiment, X is uridine or a uridine analog radical. Examples of uridine analogs include, but are not limited to, deoxyuridine; 5-Methyluridine; 3′-azido-2′,3′-dideoxy-5-chlorouridine (AzddClU); 3′-azido-2′,3′-dideoxy-5-ethyluridine (AzddEtU); 3′-azido-2′,3′-dideoxyuridine (AzddU); 3′-fluoro-2′,3′-dideoxy-5-bromouridine (3′FddBrU); 3′-fluoro-2′,3′-dideoxy-5-ethyluridine (3′FddEtU); 3′-azido-2′,3′-dideoxy-5-bromouridine (AzddBrU); 3′-azido-2′,3′-dideoxyuridine (AzddIU); 3′-fluoro-2′,3′-dideoxy-5-chlorouridine (FddClU); 3′-fluoro-2′,3′-dideoxyuridine (3′FddU); 2′,3′-dideoxy-3′-azidouridine; and 2′,3′-dideoxy-3′-3′-fluoro-5-chlorouridine. In another embodiment, X is uridine.

In another embodiment, X is adenosine or an adenosine analog radical. Example of adenosine analogs include, but are not limited to, deoxyadenosine; 2′,3′-dideoxyadenosine; 2′,3′-dideoxy-2′-fluoro-ara-adenosine; 2-chlorodeoxyadenosine; 9-(4-hydroxy-1′,2′-butadienyl)adenine; 9-(2-phosphonomethoxyethyl)adenine; 2′,3′-didehydro-2′,3′-dideoxyadenosine (D4A); dideoxyadenosine (ddA); 5-methyl-2′,3′-dideoxyadenosine (ddMeA); 3′-fluoro-2′,3′-dideoxy-arabinofuranosyl-adenine (3-Fddara-A); 3′-fluoro-2′,3′-dideoxyadenosine (3-FddA); 2′,3′-dideoxy-2′,3′-didehydro-N6-(O-methylbenzyl)adenosine; 2′,3′-dideoxy-2′,3′-didehydro-N6-(2-methylpropyl)adenosine; and 2′,3′-dideoxy-3′-fluoroadenosine. In another embodiment, X is adenosine.

In another embodiment, X is guanosine or a guanosine analog radical. Examples of guanosine analogs include, but are not limited to, of deoxyguanosine; 2′,3′-dideoxyguanosine; 2′,3′-didehydroguanosine; 3′-azido-3′-deoxyguanosine; 3′-fluoro-2′,3′-dideoxyguanosine; dideoxyguanosine (ddG); 3′-azideo-2′,3′-dideoxyguanosine (3-N.sub.3 ddG); 3′-fluoro-2′,3′-dideoxyguanosine (3-FddG); and 2′,3′-dideoxy-3′-azidoguanosine. In another embodiment, X is guanosine.

In yet another embodiment, X is thymidine or a thymidine analog radical. Examples of thymidine analogs include, but are not limited to, deoxythymidine; 3′-deoxythymidine; 2′,3′-dideoxythymidine; 2′,3′-didehydrothymidine; 3′-azido-3′-deoxythymidine; 3′-fluoro-3′-deoxythymidine; 3′-fluoro-2′,3′-dideoxythymidine

(3′FddT); 3′-deoxy-2′,3′-didehydrothymidine; and 2′,3′-didehydro-2′,3′-dideoxythymidine (D4T). In another embodiment, X is thymidine.

In yet another embodiment, X is inosine or an inosine analog radical. Examples of inosine analogs include, but are not limited to, deoxyinosine; dideoxyinosine (ddI); and 2′,3′-dideoxyinosine. In another embodiment, X is inosine.

In some embodiments X is a radical of 2,6-diaminopurine-2′,3′-dideoxyriboside; 2,6-diaminopurine-3′-azido-2′,3′-dideoxyriboside; 2,6-diaminopurine-3′-fluoro-2′,3′-dideoxyriboside; 3-phosphonomethoxyethyl-2,6-diaminopurine; 2,6-diaminopurine-2′,3′-dideoxyriboside (ddDAPR); 3′-azido-2′,3′-dideoxy-diaminopurine (N3 ddDAPR); 3′-fluoro-2′,3′-dideoxy-diaminopurine (3-FddDAPR); or 2′,3′-dideoxy-3′-fluoro-2,6-diaminopurineriboside.

In some embodiments X is a radical of Abacavir, Aciclovir, Adefovir, Alovudine, Amantadine, amprenavir, Cidofovir, Cytarabine, Desciclovir, Didanosine, Docosanol, Edoxudine, Elvucitabine, Emtricitabine, Famciclovir, Fomivirsen, Foscarnet, Ganciclovir, Idoxuridine, Lamivudine, Oseltamivir, Penciclovir, Peramivir, Rimantadine, Ribavirin, Stavudine, Tenofovir, Tenofovir, Fiacitabine, Fialuridine, doxuridine, Foscamet, Lobucavir, Sorivudine, Trifluridine, Tromantadine, ribavirine, stavudine, Valaciclovir, Valganciclovir, Vidarabine, Viramidine, Zalcitabine, Zanamivir, or Zidovudine. In some embodiments, X is alovudine, ribavirin, Zidovudine, Viramidine or Elvucitabine.

Illustrative compounds of formula (B-I-A) includes compounds of formula (B-II-A), (B-III-A), (B-IV-A), (B-V-A), (B-VI-A), (B-VII-A), (B-VIII-A), (B-IX-A), (B-X-A), (B-XI-A), (B-XII-A), (B-XIII-A) and (B-XIV-A):

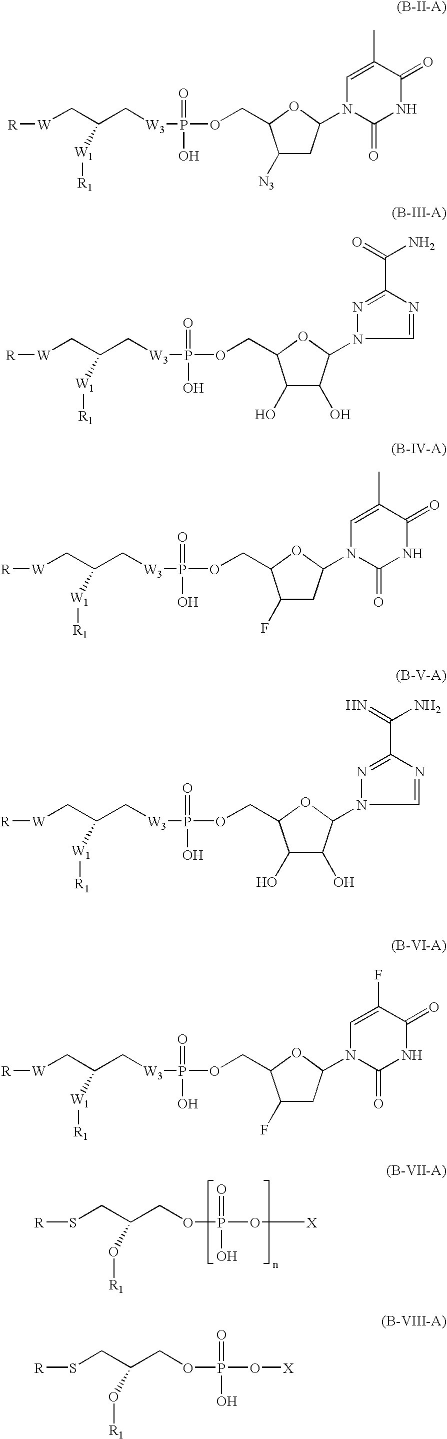

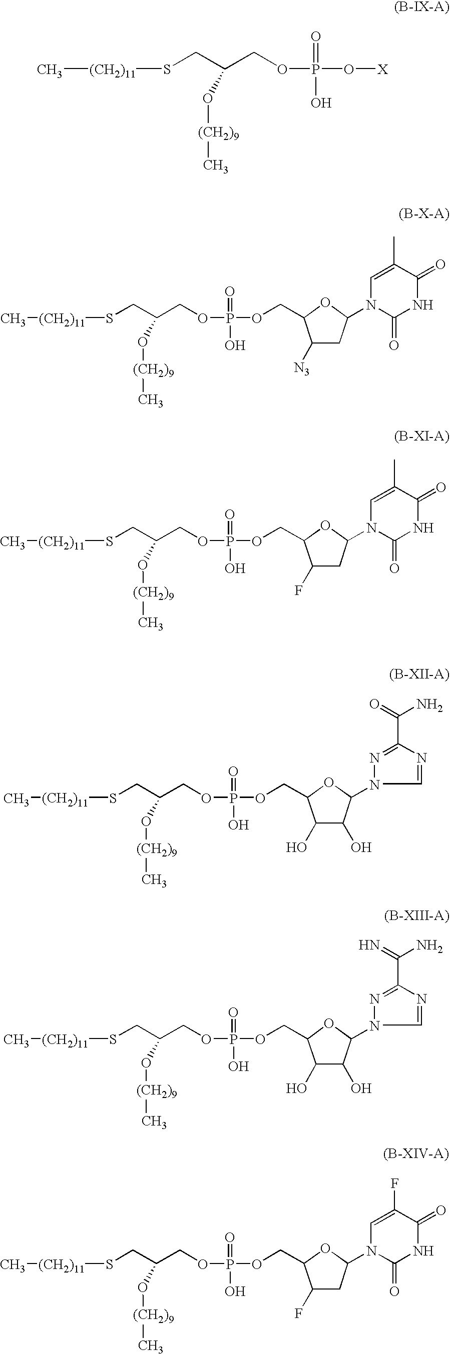

wherein W, W₁, W₃, R and R₁ are defined as described above in formula (B-I-A).

Also described herein are compounds of formula (C-VII):

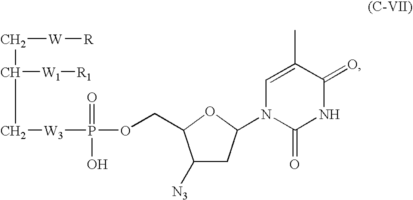

wherein

### W is —S—, —S(O)—, —O—, —S—C═C—, —SC(O)—, —O—C═C— or —OC(O)—;

### W₁ is —S—, —S(O)—, —O—, —S—C═C—, —SC(O)—, —O—C═C— or —OC(O)—;

W₃ is —O— or a covalent bond;
R is C1-C7, C8-C12 or C19-C24 cyclic, straight-chained or branched, unsaturated or saturated alkyl; and
R₁ is C1, C2, C3-C10, C11-C15 or C18-C24 cyclic, straight-chained or branched, unsaturated or saturated alkyl.

In some embodiments, W is —S— or —S(O)—.

In further or additional embodiments, W is —O—.

In some embodiments, W₁ is —S— or —S(O)—.

In further or additional embodiments, W₁ is —O—.

In some embodiments, W₃ is —O—.

In further or additional embodiments, W₃ is a covalent bond.

In some embodiments, R is unsubstituted.

In further or additional embodiments, R is substituted.

In some embodiments, R is C1 alkyl. In further or additional embodiments, R is C2 alkyl. In further or additional embodiments, R is C3 alkyl. In further or additional embodiments, R is C4 alkyl. In further or additional embodiments, R is C5 alkyl. In further or additional embodiments, R is C6 alkyl. In further or additional embodiments, R is C7 alkyl. In further or additional embodiments, R is C1-C7 alkyl. In further or additional embodiments, R is C8 alkyl. In further or additional embodiments, R is C9 alkyl. In further or additional embodiments, R is C10 alkyl. In further or additional embodiments, R is C11 alkyl. In further or additional embodiments, R is C12 alkyl. In further or additional embodiments, R is C8-C12 alkyl. In further or additional embodiments, R is C19 alkyl. In further or additional embodiments, R is C20 alkyl. In further or additional embodiments, R is C21 alkyl. In further or additional embodiments, R is C22 alkyl. In further or additional embodiments, R is C23 alkyl. In further or additional embodiments, R is C24 alkyl. In further or additional embodiments, R is C19-C24 alkyl.

In further or additional embodiments, R is substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonyl groups.

In some embodiments R is a C8-C12 cyclic, straight-chained or branched, unsaturated or saturated alkyl which can be optionally substituted.

In some embodiments R is a C12 straight-chained or branched, unsaturated or saturated alkyl which can be optionally substituted.

In further or additional embodiments, R₁ is unsubstituted.

In further or additional embodiments, R₁ is substituted.

In some embodiments, R₁ is C1 alkyl. In further or additional embodiments, R₁ is C2 alkyl. In further or additional embodiments, R₁ is C3 alkyl. In further or additional embodiments, R₁ is C4 alkyl. In further or additional embodiments, R₁ is C5 alkyl. In further or additional embodiments, R₁ is C6 alkyl. In further or additional embodiments, R₁ is C7 alkyl. In further or additional embodiments, R₁ is C8 alkyl. In further or additional embodiments, R₁ is C9 alkyl. In further or additional embodiments, R₁ is C10 alkyl. In further or additional embodiments, R₁ is C3-C10 alkyl. In some embodiments, R₁ is C11 alkyl. In further or additional embodiments, R₁ is C12 alkyl. In further or additional embodiments, R₁ is C13 alkyl. In further or additional embodiments, R₁ is C14 alkyl. In further or additional embodiments, R₁ is C15 alkyl. In further or additional embodiments, R₁ is C11-C15 alkyl. In some embodiments, R₁ is C18 alkyl. In further or additional embodiments, R₁ is C19 alkyl. In further or additional embodiments, R₁ is C20 alkyl. In further or additional embodiments, R₁ is C21 alkyl. In further or additional embodiments, R₁ is C22 alkyl. In further or additional embodiments, R₁ is C23 alkyl. In further or additional embodiments, R₁ is C24 alkyl. In further or additional embodiments, R₁ is C18-C24 alkyl.

In further or additional embodiments, R₁ is substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonyl groups.

In some embodiments, R₁ is a C1, C2, or C3-C10 cyclic, straight-chained or branched, unsaturated or saturated alkyl which can be optionally substituted.

In some embodiments R₁ is a C2 unsaturated or saturated alkyl which can be optionally substituted.

In some embodiments, W is —S— or —S(O)—; R is a C8-C12 cyclic, straight-chained or branched, unsaturated or saturated alkyl which can be optionally substituted and R₁ is a C1, C2, or C3-C10 cyclic, straight-chained or branched, unsaturated or saturated alkyl which can be optionally substituted.

In some embodiments, W is —S— or —S(O)—; R is a C12 straight-chained or branched, unsaturated or saturated alkyl which can be optionally substituted and R₁ is a C2 unsaturated or saturated alkyl which can be optionally substituted.

In some embodiments, W is —S— or —S(O)—; R is a C8-C12 cyclic, straight-chained or branched, unsaturated or saturated alkyl which can be optionally substituted and R₁ is C1-C15 cyclic, straight-chained or branched, unsaturated or saturated alkyl which can be optionally substituted.

In some embodiments, W is —S— or —S(O)—; R is a C8-C12 cyclic, straight-chained or branched, unsaturated or saturated alkyl which can be optionally substituted and R₁ is C18-C24 cyclic, straight-chained or branched, unsaturated or saturated alkyl which can be optionally substituted.

In some embodiments, W is —S— or —S(O)—; R is a C1-C7 cyclic, straight-chained or branched, unsaturated or saturated alkyl which can be optionally substituted and R₁ is C1, C2, or C3-C10cyclic, straight-chained or branched, unsaturated or saturated alkyl which can be optionally substituted.

In some embodiments, W is —S— or —S(O)—; R is a C1-C7 cyclic, straight-chained or branched, unsaturated or saturated alkyl which can be optionally substituted and R₁ is C11-C15 cyclic, straight-chained or branched, unsaturated or saturated alkyl which can be optionally substituted.

In some embodiments, W is —S— or —S(O)—; R is a C1-C7 cyclic, straight-chained or branched, unsaturated or saturated alkyl which can be optionally substituted and R₁ is C18-C24 cyclic, straight-chained or branched, unsaturated or saturated alkyl which can be optionally substituted.

In some embodiments, W is —S— or —SO; R is a C19-C24 cyclic, straight-chained or branched, unsaturated or saturated alkyl which can be optionally substituted and R₁ is C1, C2, or C3-C10 cyclic, straight-chained or branched, unsaturated or saturated alkyl which can be optionally substituted.

In some embodiments, W is —S— or —S(O)—; R is a C19-C24 cyclic, straight-chained or branched, unsaturated or saturated alkyl which can be optionally substituted and R₁ is C11-C15 cyclic, straight-chained or branched, unsaturated or saturated alkyl which can be optionally substituted.

In some embodiments, W is —S— or —S(O)—; R is a C19-C24 cyclic, straight-chained or branched, unsaturated or saturated alkyl which can be optionally substituted and R₁ is C18-C24 cyclic, straight-chained or branched, unsaturated or saturated alkyl which can be optionally substituted.

The compounds of general formula (C-I) contain asymmetrical carbon atoms All optically-active forms and racemic mixtures of these compounds are also the subject of the present invention.

### Compositions

In one aspect the invention provides compositions comprising a compound of general formula (A-I). In another aspect the invention provides compositions comprising a compound of general formula (A-I) and another agent, e.g., nucleoside or a nucleoside analog radical. In some embodiments, a compound of general formula (A-I) is co-administered with a nucleoside or a nucleoside analog.

In some embodiments, the compositions of the invention are useful for inhibiting viral replication. In some embodiments the compositions of the invention are useful for the treatment of viral infections. In some embodiments, the compositions of the invention are useful for the treatment of infections which are caused by DNA viruses, such as e.g. herpes simplex virus, the cytomegalovirus, Papovavirus, the varicella zoster virus or Epstein-Barr virus. In some embodiments, the compositions of the invention are useful for the treatment of infections which are caused by RNA viruses, such as togaviruses or retroviruses. In some embodiments, the compositions of the invention are useful for the treatment of infections which are caused by HTLV-I and II. In some embodiments, the compositions of the invention are useful for the treatment of infections which are caused by lentiviruses. In some embodiments, the compositions of the invention are useful for the treatment of infections which are caused by HIV-1 and 2.

In some embodiments, the invention provides compositions comprising one or more compounds of general formula (A-I) and pharmaceutically acceptable salts thereof. In some embodiments, the invention provides a composition comprising at least about 1, 5, 10, 20, 30, 40, 50, 60, 70, 80, 90, 95, 99, 99.5, 99.9, or 99.99% by weight of the compounds of general formula (A-I) or a pharmaceutically acceptable salt thereof. In some embodiments, the invention provides a composition for the oral delivery of the compounds of general formula (A-I) comprising no more than about 2, 5, 10, 20, 30, 40, 50, 60, 70, 80, 90, 95, 99, 99.5, 99.9, 99.99, or 100% of the compounds of general formula (A-I). In some embodiments, the invention provides a composition comprising about 1-100% of the compounds of general formula (A-I), or about 10-100% of the compounds of general formula (A-I), or about 20-100% of the compounds of general formula (A-I), or about 50-100% of the compounds of general formula (A-I), or about 80-100% of the compounds of general formula (A-I), or about 90-100% of the compounds of general formula (A-I), or about 95-100% of the compounds of general formula (A-I), or about 99-100% of the compounds of general formula (A-I). In some embodiments, the invention provides a composition comprising about 1-90% of the compounds of general formula (A-I), or about 10-90% of the compounds of general formula (A-I), or about 20-90% of the compounds of general formula (A-I), or about 50-90% of the compounds of general formula (A-I), or about 80-90% of the compounds of general formula (A-I). In some embodiments, the invention provides a composition comprising about 1-75% of the compounds of general formula (A-I), or about 10-75% of the compounds of general formula (A-I), or about 20-75% of the compounds of general formula (A-I), or about 50-75% of the compounds of general formula (A-I). In some embodiments, the invention provides a composition comprising about 1-50% of the compounds of general formula (A-I), or about 10-50% of the compounds of general formula (A-I), or about 20-50% of the compounds of general formula (A-I), or about 30-50% of the compounds of general formula (A-I), or about 40-50% of the compounds of general formula (A-I). In some embodiments, the invention provides a composition comprising about 1-40% of the compounds of general formula (A-I), or about 10-40% of the compounds of general formula (A-I), or about 20-40% of the compounds of general formula (A-I), or about 30-40% of the compounds of general formula (A-I). In some embodiments, the invention provides a composition comprising about 1-30% of the compounds of general formula (A-I), or about 10-30% of the compounds of general formula (A-I), or about 20-30% of the compounds of general formula (A-I). In some embodiments, the invention provides a composition comprising about 1-20% of the compounds of general formula (A-I), or about 10-20% of the compounds of general formula (A-I). In some embodiments, the invention provides a composition comprising about 1-10% of the compounds of general formula (A-I). In some embodiments, the invention provides a composition comprising about 1, 2, 5, 10, 20, 30, 40, 50, 60, 70, 80, 90, 95, 96, 97, 98, or 99% of the compounds of general formula (A-I).

In some of these embodiments, a pharmaceutically acceptable excipient is also included.

In some embodiments, the invention provides a composition comprising one or more of the compounds of general formula (A-I) and pharmaceutically acceptable salts thereof. In some embodiments, the amount of one or more of the compound of general formula (A-I) is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% by weight.

In some embodiments, the concentration of one or more of the compounds of general formula (A-I) or a pharmaceutically acceptable salt thereof, is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% by weight.

In some embodiments, the amount of one or more compounds of general formula (A-I) or a pharmaceutically acceptable salt thereof is in a range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% by weight.

In some embodiments, the amount of one or more of the compounds of general formula (A-I) or a pharmaceutically acceptable salt thereof is in a range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% by weight.

In some embodiments, the amount of one or more of the compounds of general formula (A-I) or a pharmaceutically acceptable salt thereof present in the composition is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more of the compounds of general formula (A-I), or a pharmaceutically acceptable salt thereof, present in the composition is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more of the compounds of general formula (A-I), or a pharmaceutically acceptable salt thereof, present in the composition is in a range of about 0.0001-10 g, 0.0005-9 g, 0.001-8 g, 0.005-7 g, 0.01-6 g, 0.05-5 g, 0.1-4 g, 0.5-4 g, or 1-3 g.

In some embodiments the invention provides pharmaceutical compositions that further include a pharmaceutically acceptable excipient. In some embodiments, the pharmaceutical compositions are administered orally. In some embodiments, the pharmaceutical compositions are administered transdermally. In some embodiments, the pharmaceutical compositions are injection. Other forms of administration are also compatible with embodiments of the pharmaceutical compositions of the invention, as described herein.

In some embodiments, the invention provides compositions containing a combination of compound of general formula (A-I) and another agent, such as a nucleoside or a nucleoside analog. Example of agents include but are not limited to nucleosides, nucleosides analogs, interferons such as α, β or γ-interferon, renal excretion inhibitors such as probenecid, nucleoside transport inhibitors such as dipyridamole, immunomodulators such as interleukin II (IL2) and granulocyte macrophage colony stimulating factor (GM-CSF), erythropoetin, empligen, thymomudulin, thymopentin, foscarnet, ribavirin and inhibitors of HIV binding to CD4 receptors e.g. soluble CD4, CD4 fragments, CD4 hybrid molecules, glycosylation inhibitors such as 2-deoxy-D-glucose, castanospermine and 1-deoxynojirimycin.

In some embodiments the agent is a nucleoside analog. In some embodiments, the nucleoside analog is lamivudine, racivir, elvucitabine, apricitabine or emtricitabine. In some embodiments, the nucleoside analog is lamivudine. In some embodiments, the nucleoside analog is racivir. In some embodiments, the nucleoside analog is elvucitabine. In some embodiments, the nucleoside analog is apricitabine. In some embodiments, the nucleoside analog is emtricitabine.

In some embodiments, the invention provides a composition comprising a compound of general formula (A-I) as described herein and a nucleoside or nucleoside analog, e.g. lamivudine. In some embodiments, the amount of the one or more nucleoside or nucleoside analogs, e.g., lamivudine is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% by weight.

In some embodiments, the amount of one or more of the nucleoside or nucleoside analogs, e.g., lamivudine is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% by weight.

In some embodiments, the concentration of one or more of the nucleoside or nucleoside analog, e.g., lamivudine is in a range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10%.

In some embodiments, the amount of one or more of the nucleoside or nucleoside analogs, e.g., lamivudine is in a range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% by wieght.

In some embodiments, the amount of one or more of the nucleoside or nucleoside analog present in the composition, e.g., lamivudine, is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more of the nucleoside or nucleoside analog present in the composition, e.g., lamivudine, is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more of the nucleoside or nucleoside analog present in the compositions, e.g., lamivudine, is in a range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments, the molar ratio of one or more of the compound of general formula (A-I) to the nucleoside or nucleoside analog, e.g. lamivudine, can be 0.0001:1 to 1:1. Without limiting the scope of the invention, the molar ratio of one or more of the Compound of general formula (A-I) to the nucleoside or nucleoside analog, e.g. lamivudine can be about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1.

Without limiting the scope of the present invention, the molar ratio of one or more of the Compound of general formula (A-I) to the nucleoside or nucleoside analog can be about 0.03×10⁻⁵:1, 0.1×10⁻⁵: 1, 0.04×10⁻³: 1, 0.03×10⁵:1, 0.02×10⁻⁵:1, 0.01×10⁻³:1, 0.1×10⁻³:1, 0.15×10⁻³:1, 0.2×10⁻³:1, 0.3×10⁻³:1, 0.4×10⁻³:1, 0.5×10⁻³:1, 0.15×10⁻²:1, 0.1×10⁻²:1, 0.2×10⁻²:1, 0.3×10⁻²:1, 0.4×10⁻²:1, 0.5×10⁻²:1, 0.6×10⁻²:1, 0.9×10⁻²:1, 0.01:1, 0.1:1; or 0.2:1 per dose.

Without limiting the scope of the present invention, the molar ratio of one or more of the compound of general formula (A-I) to the nucleoside or nucleoside analog, e.g. lamivudine, can be about 0.001:1, 0.002:1, 0.003:1, 0.004:1, 0.005:1, 0.006:1, 0.007:1, 0.008:1, 0.009:1, 0.01:1, 0.02:1, 0.03:1, 0.04:1, 0.05:1, 0.06:1, 0.07:1, 0.08:1, 0.09:1, 0.1:1, 0.2:1, 0.3:1, 0.4:1, 0.5:1, 0.6:1, 0.7:1, 0.8:1, 0.9:1, 1:1, 2:1, 3:1, 4:1, or 5:1 per dose.

In some embodiments, the invention provides a composition comprising at least about 1, 5, 10, 20, 30, 40, 50, 60, 70, 80, 90, 95, 99, 99.5, 99.9, or 99.99% by weight of the compound of general formula (B-I) or (B-I-A) or pharmaceutically acceptable salts thereof. In some embodiments, the invention provides a composition for the oral delivery of the compounds of general formula (B-I) or (B-I-A) or pharmaceutically acceptable salts thereof comprising no more than about 2, 5, 10, 20, 30, 40, 50, 60, 70, 80, 90, 95, 99, 99.5, 99.9, 99.99, or 100% of the compounds of general formula (B-I) or (B-I-A). In some embodiments, the invention provides a composition comprising about 1-100% of the compounds of general formula (B-I) or (B-I-A), or about 10-100% of the compounds of general formula (B-I) or (B-I-A), or about 20-100% of the compounds of general formula (B-I) or (B-I-A), or about 50-100% of the compounds of general formula (B-I) or (B-I-A), or about 80-100% of the compounds of general formula (B-I) or (B-I-A), or about 90-100% of the compounds of general formula (B-I) or (B-I-A), or about 95-100% of the compounds of general formula (B-I) or (B-I-A), or about 99-100% of the compounds of general formula (B-I) or (B-I-A). In some embodiments, the invention provides a composition comprising about 1-90% of the compounds of general formula (B-I) or (B-I-A), or about 10-90% of the compounds of general formula (B-I) or (B-I-A), or about 20-90% of the compounds of general formula (B-I) or (B-I-A), or about 50-90% of the compounds of general formula (B-I) or (B-I-A), or about 80-90% of the compounds of general formula (B-I) or (B-I-A). In some embodiments, the invention provides a composition comprising about 1-75% of the compounds of general formula (B-I) or (B-I-A), or about 10-75% of the compounds of general formula (B-I) or (B-I-A), or about 20-75% of the compounds of general formula (B-I) or (B-I-A), or about 50-75% of the compounds of general formula (B-I) or (B-I-A). In some embodiments, the invention provides a composition comprising about 1-50% of the compounds of general formula (B-I) or (B-I-A), or about 10-50% of the compounds of general formula (B-I) or (B-I-A), or about 20-50% of the compounds of general formula (B-I) or (B-I-A), or about 30-50% of the compounds of general formula (B-I) or (B-I-A), or about 40-50% of the compounds of general formula (B-I) or (B-I-A). In some embodiments, the invention provides a composition comprising about 1-40% of the compounds of general formula (B-I) or (B-I-A), or about 10-40% of the compounds of general formula (B-I) or (B-I-A), or about 20-40% of the compounds of general formula (B-A) or (B-I-A), or about 30-40% of the compounds of general formula (B-I) or (B-I-A). In some embodiments, the invention provides a composition comprising about 1-30% of the compounds of general formula (B-I) or (B-I-A), or about 10-30% of the compounds of general formula (B-I) or (B-I-A), or about 20-30% of the compounds of general formula (B-I) or (B-I-A). In some embodiments, the invention provides a composition comprising about 1-20% of the compounds of general formula (B-I) or (B-I-A), or about 10-20% of the compounds of general formula (B-I) or (B-I-A). In some embodiments, the invention provides a composition comprising about 1-10% of the compounds of general formula (B-I) or (B-I-A). In some embodiments, the invention provides a composition comprising about 1, 2, 5, 10, 20, 30, 40, 50, 60, 70, 80, 90, 95, 96, 97, 98, or 99% of the compounds of general formula (B-I) or (B-I-A).

In some of these embodiments, a pharmaceutically acceptable excipient is also included.

In one aspect the invention provides compositions comprising a compound of general formula (B-I) or (B-I-A) as described herein. In another aspect the invention provides compositions comprising a compound of general formula (B-I) or (B-I-A) and another agent. In another aspect the invention provides compositions comprising a compound of general formula (B-I) or (B-I-A) and a nucleoside or a nucleoside analog. In some embodiments, a compound of general formula (B-I) or (B-I-A) is co-administered with a nucleoside or a nucleoside analog. Co-administration encompasses administration of two or more agents to a subject so that both agents and/or their metabolites are present in the subject at the same time. Co-administration includes simultaneous administration in separate compositions, administration at different times in separate compositions, or administration in a composition in which both agents are present.

In some embodiments, the compositions of the invention are useful for inhibiting viral replication. In some embodiments the compositions of the invention are useful for the treatment of viral infections. In some embodiments, the compositions of the invention are useful for the treatment of infections which are caused by DNA viruses, such as e.g. herpes simplex virus, the cytomegalovirus, Papovavirus, the varicella zoster virus or Epstein-Barr virus. In some embodiments, the compositions of the invention are useful for the treatment of infections which are caused by RNA viruses, such as togaviruses or retroviruses. In some embodiments, the compositions of the invention are useful for the treatment of infections which are caused by HTLV-I and II. In some embodiments, the compositions of the invention are useful for the treatment of infections which are caused by lentiviruses. In some embodiments, the compositions of the invention are useful for the treatment of infections which are caused by HIV-1 and 2.

In some embodiments, the invention provides a composition comprising one or more of the compounds of general formula (B-I) or (B-I-A) or a pharmaceutically acceptable salt thereof. In some embodiments, the amount of one or more of the compound of general formula (B-I) or (B-I-A) is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% by weight.

In some embodiments, the amount of one or more of the compounds of general formula (B-I) or (B-I-A) or a pharmaceutically acceptable salt thereof, is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% by weight.

In some embodiments, the amount of one or more compounds of general formula (B-I) or (B-I-A) or a pharmaceutically acceptable salt thereof is in a range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10%.

In some embodiments, the amount or a pharmaceutically acceptable salt thereof of one or more of the compounds of general formula (B-I) or (B-I-A) or a pharmaceutically acceptable salt thereof is in a range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% by weight.

In some embodiments, the amount of one or more of the compounds of general formula (B-I) or (B-I-A) or a pharmaceutically acceptable salt thereof present in the composition is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more of the compounds of general formula (B-I) or (B-I-A) or a pharmaceutically acceptable salt thereof present in the composition is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more of the compounds of general formula (B-I) or (B-I-A) or a pharmaceutically acceptable salt thereof present in the composition is in a range of about 0.0001-10 g, 0.0005-9 g, 0.001-8 g, 0.005-7 g, 0.01-6 g, 0.05-5 g, 0.1-4 g, 0.5-4 g, or 1-3 g.

In some embodiments the invention provides pharmaceutical compositions that further include a pharmaceutically acceptable excipient. In some embodiments, the pharmaceutical compositions are administered orally. In some embodiments, the pharmaceutical compositions are administered trandermally. In some embodiments, the pharmaceutical compositions are injected. Other forms of administration are also compatible with embodiments of the pharmaceutical compositions of the invention, as described herein

In some embodiments, the invention provides compositions containing a combination of compound of general formula (B-I) or (B-I-A) and another agent, such as a nucleoside or a nucleoside analog. Example of agents that can be used in combination with compounds of general formula (B-I) or (B-I-A) include but are not limited to nucleosides, nucleosides analogs, interferons such as α, β or γ-interferon, renal excretion inhibitors such as probenecid, nucleoside transport inhibitors such as dipyridamole, immunomodulators such as interleukin II (IL2) and granulocyte macrophage colony stimulating factor (GM-CSF), erythropoetin, empligen, thymomudulin, thymopentin, foscarnet, ribavirin and inhibitors of HIV binding to CD4 receptors e.g. soluble CD4, CD4 fragments, CD4 hybrid molecules, glycosylation inhibitors such as 2-deoxy-D-glucose, castanospernine and 1-deoxynojirimycin.

In some embodiments the agent is a nucleoside analog. In some embodiments, the nucleoside analog is lamivudine, racivir, elvucitabine, apricitabine or emtricitabine. In some embodiments, the nucleoside analog is lamivudine. In some embodiments, the nucleoside analog is racivir. In some embodiments, the nucleoside analog is elvucitabine. In some embodiments, the nucleoside analog is apricitabine. In some embodiments, the nucleoside analog is emtricitabine.

In some embodiments, the invention provides a composition comprising a compound of general formula (B-I) or (B-I-A) or a pharmaceutically acceptable salt thereof as described herein and a nucleoside or nucleoside analog, e.g. lamivudine. In some embodiments, the amount of one or more nucleoside or nucleoside analog, e.g., lamivudine is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% by weight.

In some embodiments, the amount of one or more of the nucleoside or nucleoside analog, e.g., lamivudine is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% by weight.

In some embodiments, the amount of one or more of the nucleoside or nucleoside analog, e.g., lamivudine is in a range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% by weight.

In some embodiments, the amount of one or more of the nucleoside or nucleoside analog, e.g., lamivudine is in a range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% by weight.

In some embodiments, the amount of one or more of the nucleoside or nucleoside analog present in the composition, e.g., lamivudine, is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more of the nucleoside or nucleoside analog present in the composition, e.g., lamivudine, is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more of the nucleoside or nucleoside analog present in the compositions, e.g., lamivudine, is in a range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments, the molar ratio of one or more of the compound of general formula (B-I) or (B-I-A) to the nucleoside or nucleoside analog, e.g. lamivudine, can be 0.0001:1 to 1:1. Without limiting the scope of the invention, the molar ratio of one or more of the compound of formula (B-I) or (B-I-A) to the nucleoside or nucleoside analog, e.g. lamivudine can be about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1.

Without limiting the scope of the present invention, the molar ratio of one or more of the compound of general formula (B-I) or (B-I-A) to the nucleoside or nucleoside analog can be about 0.03×10⁻⁵:1, 0.1×10⁻⁵:1, 0.04×10⁻³:1, 0.03×10⁻⁵:1, 0.02×10⁻⁵:1, 0.01×10⁻³:1, 0.1×10⁻³:1, 0.15×10⁻³:1, 0.2×10⁻³:1, 0.3×10⁻³:1, 0.4×10⁻³:1, 0.5×10⁻³:1, 0.15×10⁻²:1, 0.1×10⁻²:1, 0.2×10⁻²:1, 0.3×10⁻²:1, 0.4×10⁻²:1, 0.5×10⁻²:1, 0.6×10⁻²:1, 0.8×10⁻²:1, 0.01:1, 0.1:1; or 0.2:1 per dose.

Without limiting the scope of the present invention, the molar ratio of one or more of the compound of general formula (B-I) or (B-I-A) to the nucleoside or nucleoside analog, e.g. lamivudine, can be about 0.001:1, 0.002:1, 0.003:1, 0.004:1, 0.005:1, 0.006:1, 0.007:1, 0.008:1, 0.009:1, 0.01:1, 0.02:1, 0.03:1, 0.04:1, 0.05:1, 0.06:1, 0.07:1, 0.08:1, 0.09:1, 0.1:1, 0.2:1, 0.3:1, 0.4:1, 0.5:1, 0.6:1, 0.7:1, 0.8:1, 0.9:1, 1:1, 2:1, 3:1, 4:1, or 5:1 per dose.

In some embodiments, the invention provides compositions comprising a compound of formula (C-VII) or a pharmaceutically acceptable salt thereof. In some embodiments the compositions comprise a compound of formula (C-VII) and a nucleoside or a nucleoside analog. In some embodiments, the nucleoside or nucleoside analog is cytidine, a cytidine analog, uridine, a uridine analog, adenosine, an adenosine analog, guanosine, a guanosine analog, thymidine, a thymidine analog, inosine or an inosine analog.

In some embodiments, the invention provides a composition comprising a compound of formula (C-VII) or a pharmaceutically acceptable salt thereof and cytidine or a cytidine analog. Examples of cytidine analogs include, but are not limited to, deoxycytidine; 2′,3′-dideoxycytidine; 2′,3′-didehydrocytidine carbocyclic; 2′,3′-didehydro-2′,3′-dideoxycytidine (D4C); 2′,3′-didehydro-2′,3′-dideoxy-5-methylcytidine (D4MeC); fluoro-2′,3′-dideoxycytidine (5-F-ddC); 3-(4-hydroxy-1′,2′-butadienyl)cytosine; 3′-azido-2′,3′-dideoxy-5-methylcytosine (AzddMeC); 3′-azido-2′,3′-dideoxy-5-methylcytosine (AzddMeC); 3′-azido-2′,3′-dideoxy-5-methylcytosine-N4-OH (AzddMeC N4-OH); 3′-azido-2′,3′-dideoxy-5-methylcytosine-N4Me, (AzddMeC N4Me); 3′-azido-2′,3′-dideoxycytosine (AzddC); 3′-azido-2′,3′-dideoxy-5-fluorocytosine (AzddFC); 2′,3′-dideoxy-2′,3′-didehydrocytidine; and beta-L-5-fluoro-2′,3′-dideoxy-2′,3′-didehydrocytidine.

In some embodiments, the invention provides a composition comprising a compound of formula (C-VII) or a pharmaceutically acceptable salt thereof and uridine or a uridine analog. Examples of uridine analogs include, but are not limited to, deoxyuridine; 5-Methyluridine; 3′-azido-2′,3′-dideoxy-5-chlorouridine (AzddClU); 3′-azido-2′,3′-dideoxy-5-ethyluridine (AzddEtU); 3′-azido-2′,3′-dideoxyuridine (AzddU); 3′-fluoro-2′,3′-dideoxy-5-bromouridine (3′FddBrU); 3′-fluoro-2′,3′-dideoxy-5-ethyluridine (3′FddEtU); 3′-azido-2′,3′-dideoxy-5-bromouridine (AzddBrU); 3′-azido-2′,3′-dideoxyuridine (AzddIU); 3′-fluoro-2′,3′-dideoxy-5-chlorouridine (FddClU); 3′-fluoro-2′,3′-dideoxyuridine (3′FddU); 2′,3′-dideoxy-3′-azidouridine; and 2′,3′-dideoxy-3′-3′-fluoro-5-chlorouridine.

In some embodiments, the invention provides a composition comprising a compound of formula (C-VII) or a pharmaceutically acceptable salt thereof and adenosine or an adenosine analog. Example of adenosine analogs include, but are not limited to, deoxyadenosine; 2′,3′-dideoxyadenosine; 2′,3′-dideoxy-2′-fluoro-ara-adenosine; 2-chlorodeoxyadenosine; 9-(4-hydroxy-1′,2′-butadienyl)adenine; 9-(2-phosphonomethoxyethyl)adenine; 2′,3′-didehydro-2′,3′-dideoxyadenosine (D4A); dideoxyadenosine (ddA); 5-methyl-2′,3′-dideoxyadenosine (ddMeA); 3′-fluoro-2′,3′-dideoxy-arabinofuranosyl-adenine (3-Fddara-A); 3′-fluoro-2′,3′-dideoxyadenosine (3-FddA); 2′,3′-dideoxy-2′,3′-didehydro-N6-(O-methylbenzyl)adenosine; 2′,3′-dideoxy-2′,3′-didehydro-N6-(2-methylpropyl)adenosine; and 2′,3′-dideoxy-3′-fluoroadenosine.

In some embodiments, the invention provides a composition comprising a compound of formula (C-VII) or a pharmaceutically acceptable salt thereof and guanosine or a guanosine analog. Examples of guanosine analogs include, but are not limited to, of deoxyguanosine; 2′,3′-dideoxyguanosine; 2′,3′-didehydroguanosine; 3′-azido-3′-deoxyguanosine; 3′-fluoro-2′,3′-dideoxyguanosine; dideoxyguanosine (ddG); 3′-azideo-2′,3′-dideoxyguanosine (3-N.sub.3 ddG); 3′-fluoro-2′,3′-dideoxyguanosine (3-FddG); and 2′,3′-dideoxy-3′-azidoguanosine. In another embodiment, X is guanosine.

In some embodiments, the invention provides a composition comprising a compound of formula (C-VII) or a pharmaceutically acceptable salt thereof and thymidine or a thymidine analog. Examples of thymidine analogs include, but are not limited to, deoxythymidine; 3′-deoxythymidine; 2′,3′-dideoxythymidine; 2′,3′-didehydrothymidine; 3′-azido-3′-deoxythymidine; 3′-fluoro-3′-deoxythymidine; 3′-fluoro-2′,3′-dideoxythymidine (3′FddT); 3′-deoxy-2′,3′-didehydrothymidine; and 2′,3′-didehydro-2′,3′-dideoxythymidine (D4T). In another embodiment, X is thymidine.

In some embodiments, the invention provides a composition comprising a compound of formula (C-VII) or a pharmaceutically acceptable salt thereof and inosine or an inosine analog. Examples of inosine analogs include, but are not limited to, deoxyinosine; dideoxyinosine (ddI); and 2′,3′-dideoxyinosine.

In some embodiments, the invention provides a composition comprising a compound of formula (C-VII) or a pharmaceutically acceptable salt thereof and a nucleoside analog. In some embodiments, the nucleoside analog is 2,6-diaminopurine-2′,3′-dideoxyriboside; 2,6-diaminopurine-3′-azido-2′,3′-dideoxyriboside; 2,6-diaminopurine-3′-fluoro-2′,3′-dideoxyriboside; 3-phosphonomethoxyethyl-2,6-diaminopurine; 2,6-diaminopurine-2′,3′-dideoxyriboside (ddDAPR); 3′-azido-2′,3′-dideoxy-diaminopurine (N3 ddDAPR); 3′-fluoro-2′,3′-dideoxy-diaminopurine (3-FddDAPR); or 2′,3′-dideoxy-3′-fluoro-2,6-diaminopurineriboside.

In some embodiments, the invention provides a composition comprising a compound of formula (C-VII) or a pharmaceutically acceptable salt thereof and a pharmaceutically acceptable salt of the nucleoside and nucleoside analogs described above.

In some embodiments, the invention provides a composition comprising a compound of formula (C-VII) or a pharmaceutically acceptable salt thereof and Abacavir, Aciclovir, Adefovir, Alovudine, Amantadine, amprenavir, Cidofovir, Cytarabine, Desciclovir, Didanosine, Docosanol, Edoxudine, Elvucitabine, Emtricitabine, Famciclovir, Fomivirsen, Foscamet, Ganciclovir, Idoxuridine, Lamivudine, Oseltamivir, Penciclovir, Peramivir, Rimantadine, Ribavirin, Stavudine, Tenofovir, Tenofovir, Fiacitabine, Fialuridine, doxuridine, Foscamet, Lobucavir, Sorivudine, Trifluridine, Tromantadine, ribavirine, stavudine, Valaciclovir, Valganciclovir, Vidarabine, Viramidine, Zalcitabine, Zanamivir, or Zidovudine.

In some embodiments, the invention provides a composition comprising a compound of formula (C-VII) or a pharmaceutically acceptable salt thereof comprising at least about 1, 5, 10, 20, 30, 40, 50, 60, 70, 80, 90, 95, 99, 99.5, 99.9, or 99.99% by weight of a compound of formula (C-VII) or a pharmaceutically acceptable salt thereof. In some embodiments, the invention provides a composition for the oral delivery of a compound of formula (C-VII) or a pharmaceutically acceptable salt thereof comprising no more than about 2, 5, 10, 20, 30, 40, 50, 60, 70, 80, 90, 95, 99, 99.5, 99.9, 99.99, or 100% by weight of the compound of formula (C-VII) or a pharmaceutically acceptable salt thereof. In some embodiments, the invention provides a composition comprising about 1-100% of the compound of formula (C-VII), or about 10-100% of the compound of formula (C-VII), or about 20-100% of the compound of formula (C-VII), or about 50-100% of the compound of formula (C-VII), or about 80-100% of the compound of formula (C-VII), or about 90-100% of the compound of formula (C-VII), or about 95-100% of the compound of formula (C-VII), or about 99-100% of the compound of formula (C-VII). In some embodiments, the invention provides a composition comprising about 1-90% of the compound of formula (C-VII), or about 10-90% of the compound of formula (C-VII), or about 20-90% of the compound of formula (C-VII), or about 50-90% of the compound of formula (C-VII), or about 80-90% of the compound of formula (C-VII). In some embodiments, the invention provides a composition comprising about 1-75% of the compound of formula (C-VII), or about 10-75% of the compound of formula (C-VII), or about 20-75% of the compound of formula (C-VII), or about 50-75% of the compound of formula (C-VII). In some embodiments, the invention provides a composition comprising about 1-50% of the compound of formula (C-VII), or about 10-50% of the compound of formula (C-VII), or about 20-50% of the compound of formula (C-VII), or about 30-50% of the compound of formula (C-VII), or about 40-50% of the compound of formula (C-VII). In some embodiments, the invention provides a composition comprising about 1-40% of the compound of formula (C-VII), or about 10-40% of the compound of formula (C-VII), or about 20-40% of the compound of formula (C-VII), or about 30-40% of the compound of formula (C-VII). In some embodiments, the invention provides a composition comprising about 1-30% of the compound of formula (C-VII), or about 10-30% of the compound of formula (C-VII), or about 20-30% of the compound of formula (C-VII). In some embodiments, the invention provides a composition comprising about 1-20% of the compound of formula (C-VII), or about 10-20% of the compound of formula (C-VII). In some embodiments, the invention provides a composition comprising about 1-10% of the compound of formula (C-VII). In some embodiments, the invention provides a composition comprising about 1, 2, 5, 10, 20, 30, 40, 50, 60, 70, 80, 90, 95, 96, 97, 98, or 99% of the compound of formula (C-VII). Unless specified otherwise percentages refer to percent by weight.

In some embodiments, the compositions further comprise a pharmaceutically acceptable excipient.

In some embodiments, the invention provides compositions comprising a compound of structure (C-I).

The compound of structure (C-I) contains an asymmetrical carbon atom. It should be understood that all optically-active forms and racemic mixtures of the compounds are also the subject of the present invention.

In some embodiments the invention provides compositions comprising a compound of structure (C-I) and a nucleoside or a nucleoside analog. In some embodiments, the nucleoside or nucleoside analog is cytidine, a cytidine analog, uridine, a uridine analog, adenosine, an adenosine analog, guanosine, a guanosine analog, thymidine, a thymidine analog, inosine or an inosine analog. Examples of nucleosides analogs are described above.

In one aspect the invention provides compositions comprising an effective amount of a compound of structure (C-I) or a pharmaceutically acceptable salt thereof and an effective amount of a compound of structure (C-II) or a pharmaceutically acceptable salt thereof. In some embodiments the composition further comprises one or more pharmaceutically acceptable carriers.

In some embodiments, the compound of structure (C-II) is a mixture of its cis-isomers. In some embodiments, the compound of structure (C-II) is a mixture of its trans-isomers. In some embodiments, the compound of structure (C-II) is in the form of a single enantiomer. In some embodiments, the compound of structure (C-II) is in the form of the (−) enantiomer.

In one aspect the invention provides compositions comprising an effective amount of a compound of structure (C-I) or a pharmaceutically acceptable salt thereof and an effective amount of a compound of structure (C-III) or a pharmaceutically acceptable salt thereof. In some embodiments the composition further comprises one or more pharmaceutically acceptable carriers.

In one aspect the invention provides compositions comprising an effective amount of a compound of structure (C-I) or a pharmaceutically acceptable salt thereof and an effective amount of a compound of structure (C-IV) or a pharmaceutically acceptable salt thereof. In some embodiments the composition further comprises one or more pharmaceutically acceptable carriers.

In some the compound of structure (C-IV) is a mixture of its cis-isomers. In some embodiments, the compound of structure (C-IV) is the (cis) isomer in the form of a single enantiomer. In some embodiments, the compound of structure (C-IV) is the (1S,4R) isomer.

In one aspect the invention provides compositions comprising an effective amount of a compound of structure (C-I) or a pharmaceutically acceptable salt thereof and an effective amount of a compound of structure (C-V) or a pharmaceutically acceptable salt thereof. In some embodiments the composition further comprises one or more pharmaceutically acceptable carriers.

In some embodiments, the compound of structure (C-V) is a mixture of its cis-isomers. In some embodiments, the compound of structure (C-V) is a mixture of its trans-isomers. In some embodiments, the compound of structure (C-V) is in the form of a single enantiomer. In some embodiments, the compound of structure (C-V) is in the form of the (−) enantiomer.

In one aspect the invention provides compositions comprising an effective amount of a compound of structure (C-I) or a pharmaceutically acceptable salt thereof and an effective amount of a compound of structure (C-VI) or a pharmaceutically acceptable salt thereof. In some embodiments the composition further comprises one or more pharmaceutically acceptable carriers.

In some embodiments, the compound of structure (C-VI) is a mixture of its cis-isomers. In some embodiments, the compound of structure (C-VI) is a mixture of its trans-isomers. In some embodiments, the compound of structure (C-VI) is in the form of a single enantiomer. In some embodiments, the compound of structure (C-VI) is in the form of the (−) enantiomer.

In some embodiments, the invention provides a composition comprising a compound of structure (C-I), that contain at least about 1, 5, 10, 20, 30, 40, 50, 60, 70, 80, 90, 95, 99, 99.5, 99.9, or 99.99% by weight of a compound of structure (C-I). In some embodiments, the invention provides a composition for the oral delivery of a compound of structure (C-I) comprising no more than about 2, 5, 10, 20, 30, 40, 50, 60, 70, 80, 90, 95, 99, 99.5, 99.9, 99.99, or 100% a compound of structure (C-I). In some embodiments, the invention provides a composition comprising about 1-100% a compound of structure (C-I), or about 10-100% a compound of structure (C-I), or about 20-100% a compound of structure (C-I), or about 50-100% a compound of structure (C-I), or about 80-100% a compound of structure (C-I), or about 90-100% a compound of structure (C-I), or about 95-100% a compound of structure (C-I), or about 99-100% a compound of structure (C-I). In some embodiments, the invention provides a composition comprising about 1-90% a compound of structure (C-I), or about 10-90% a compound of structure (C-I), or about 20-90% a compound of structure (C-I), or about 50-90% a compound of structure (C-I), or about 80-90% a compound of structure (C-I). In some embodiments, the invention provides a composition comprising about 1-75% a compound of structure (C-I), or about 10-75% a compound of structure (C-I), or about 20-75% a compound of structure (C-I), or about 50-75% a compound of structure (C-I). In some embodiments, the invention provides a composition comprising about 1-50% a compound of structure (C-I), or about 10-50% a compound of structure (C-I), or about 20-50% a compound of structure (C-I), or about 30-50% a compound of structure (C-I), or about 40-50% a compound of structure (C-I). In some embodiments, the invention provides a composition comprising about 1-40% a compound of structure (C-I), or about 10-40% a compound of structure (C-I), or about 20-40% a compound of structure (C-I), or about 30-40% a compound of structure (C-I). In some embodiments, the invention provides a composition comprising about 1-30% a compound of structure (C-I), or about 10-30% a compound of structure (C-I), or about 20-30% a compound of structure (C-I). In some embodiments, the invention provides a composition comprising about 1-20% a compound of structure (C-I), or about 10-20% a compound of structure (C-I). In some embodiments, the invention provides a composition comprising about 1-10% a compound of structure (C-I). In some embodiments, the invention provides a composition comprising about 1, 2, 5, 10, 20, 30, 40, 50, 60, 70, 80, 90, 95, 96, 97, 98, or 99% a compound of structure (C-I). Unless otherwise specified all percentages are by weight.

In some of these embodiments, the composition further comprises a pharmaceutically acceptable excipient.

In some embodiments, the invention provides a composition comprising at least about 1, 5, 10, 20, 30, 40, 50, 60, 70, 80, 90, 95, 99, 99.5, 99.9, or 99.99% by weight of a compound of structure (C-II) or a pharmaceutically acceptable salt thereof. In some embodiments, the invention provides a composition for the oral delivery of a compound of structure (C-II) comprising no more than about 2, 5, 10, 20, 30, 40, 50, 60, 70, 80, 90, 95, 99, 99.5, 99.9, 99.99, or 100% of a compound of structure (C-II). In some embodiments, the invention provides a composition comprising about 1-100% of a compound of structure (C-II), or about 10-100% of a compound of structure (C-II), or about 20-100% of a compound of structure (C-II), or about 50-100% of a compound of structure (C-II), or about 80-100% of a compound of structure (C-II), or about 90-100% of a compound of structure (C-II), or about 95-100% of a compound of structure (C-II), or about 99-100% of a compound of structure (C-II). In some embodiments, the invention provides a composition comprising about 1-90% of a compound of structure (C-II), or about 10-90% of a compound of structure (C-II), or about 20-90% of a compound of structure (C-II), or about 50-90 of a compound of structure (C-II), or about 80-90% of a compound of structure (C-II). In some embodiments, the invention provides a composition comprising about 1-75 of a compound of structure (C-II), or about 10-75% of a compound of structure (C-II), or about 20-75% of a compound of structure (C-II), or about 50-75% of a compound of structure (C-II). In some embodiments, the invention provides a composition comprising about 1-50% of a compound of structure (C-II), or about 10-50% of a compound of structure (C-II), or about 20-50% of a compound of structure (C-II), or about 30-50% of a compound of structure (C-II), or about 40-50% of a compound of structure (C-II). In some embodiments, the invention provides a composition comprising about 1-40% of a compound of structure (C-II), or about 10-40% of a compound of structure (C-II), or about 20-40% of a compound of structure (C-II), or about 30-40% of a compound of structure (C-II). In some embodiments, the invention provides a composition comprising about 1-30% of a compound of structure (C-II), or about 10-30% of a compound of structure (C-II), or about 20-30% of a compound of structure (C-II). In some embodiments, the invention provides a composition comprising about 1-20 of a compound of structure (C-II), or about 10-20% of a compound of structure (C-II). In some embodiments, the invention provides a composition comprising about 1-10% of a compound of structure (C-II). In some embodiments, the invention provides a composition comprising about 1, 2, 5, 10, 20, 30, 40, 50, 60, 70, 80, 90, 95, 96, 97, 98, or 99% of a compound of structure (C-II).

In some of these embodiments, the composition further comprises a pharmaceutically acceptable excipient.

In some embodiments, the invention provides a composition comprising at least about 1, 5, 10, 20, 30, 40, 50, 60, 70, 80, 90, 95, 99, 99.5, 99.9, or 99.99% of a compound of structure (C-III). In some embodiments, the invention provides a composition for the oral delivery of a compound of structure (C-III) comprising no more than about 2, 5, 10, 20, 30, 40, 50, 60, 70, 80, 90, 95, 99, 99.5, 99.9, 99.99, or 100% of a compound of structure (C-III). In some embodiments, the invention provides a composition comprising about 1-100% of a compound of structure (C-III), or about 10-100% of a compound of structure (C-III), or about 20-100% of a compound of structure (C-III), or about 50-100% of a compound of structure (C-III), or about 80-100% of a compound of structure (C-III), or about 90-100% of a compound of structure (C-III), or about 95-100% of a compound of structure (C-III), or about 99-100% of a compound of structure (C-III). In some embodiments, the invention provides a composition comprising about 1-90% of a compound of structure (C-III), or about 10-90% of a compound of structure (C-III), or about 20-90% of a compound of structure (C-III), or about 50-90 of a compound of structure (C-III), or about 80-90% of a compound of structure (C-III). In some embodiments, the invention provides a composition comprising about 1-75 of a compound of structure (C-III), or about 10-75% of a compound of structure (C-III), or about 20-75% of a compound of structure (C-III), or about 50-75% of a compound of structure (C-III). In some embodiments, the invention provides a composition comprising about 1-50% of a compound of structure (C-III), or about 10-50% of a compound of structure (C-III), or about 20-50% of a compound of structure (C-III), or about 30-50% of a compound of structure (C-III), or about 40-50% of a compound of structure (C-III). In some embodiments, the invention provides a composition comprising about 1-40% of a compound of structure (C-III), or about 10-40% of a compound of structure (C-III), or about 20-40% of a compound of structure (C-III), or about 30-40% of a compound of structure (C-III). In some embodiments, the invention provides a composition comprising about 1-30% of a compound of structure (C-III), or about 10-30% of a compound of structure (C-III), or about 20-30% of a compound of structure (C-III). In some embodiments, the invention provides a composition comprising about 1-20 of a compound of structure (C-III), or about 10-20% of a compound of structure (C-III). In some embodiments, the invention provides a composition comprising about 1-10% of a compound of structure (C-III). In some embodiments, the invention provides a composition comprising about 1, 2, 5, 10, 20, 30, 40, 50, 60, 70, 80, 90, 95, 96, 97, 98, or 99% of a compound of structure (C-III).

In some of these embodiments, the composition further comprises a pharmaceutically acceptable excipient.

In some embodiments, the invention provides a composition comprising at least about 1, 5, 10, 20, 30, 40, 50, 60, 70, 80, 90, 95, 99, 99.5, 99.9, or 99.99% of a compound of structure (C-IV). In some embodiments, the invention provides a composition for the oral delivery of a compound of structure (C-IV) comprising no more than about 2, 5, 10, 20, 30, 40, 50, 60, 70, 80, 90, 95, 99, 99.5, 99.9, 99.99, or 100% of a compound of structure (C-IV). In some embodiments, the invention provides a composition comprising about 1-100% of a compound of structure (C-IV), or about 10-100% of a compound of structure (C-IV), or about 20-100% of a compound of structure (C-IV), or about 50-100% of a compound of structure (C-IV), or about 80-100% of a compound of structure (C-IV), or about 90-100% of a compound of structure (C-IV), or about 95-100% of a compound of structure (C-IV), or about 99-100% of a compound of structure (C-IV). In some embodiments, the invention provides a composition comprising about 1-90% of a compound of structure (C-IV), or about 10-90% of a compound of structure (C-IV), or about 20-90% of a compound of structure (C-IV), or about 50-90 of a compound of structure (C-IV), or about 80-90% of a compound of structure (C-IV). In some embodiments, the invention provides a composition comprising about 1-75 of a compound of structure (C-IV), or about 10-75% of a compound of structure (C-IV), or about 20-75% of a compound of structure (C-IV), or about 50-75% of a compound of structure (C-IV). In some embodiments, the invention provides a composition comprising about 1-50% of a compound of structure (C-IV), or about 10-50% of a compound of structure (C-IV), or about 20-50% of a compound of structure (C-IV), or about 30-50% of a compound of structure (C-IV), or about 40-50% of a compound of structure (C-IV). In some embodiments, the invention provides a composition comprising about 1-40% of a compound of structure (C-IV), or about 10-40% of a compound of structure (C-IV), or about 20-40% of a compound of structure (C-IV), or about 30-40% of a compound of structure (C-IV). In some embodiments, the invention provides a composition comprising about 1-30% of a compound of structure (C-IV), or about 10-30% of a compound of structure (C-IV), or about 20-30% of a compound of structure (C-IV). In some embodiments, the invention provides a composition comprising about 1-20 of a compound of structure (C-IV), or about 10-20% of a compound of structure (C-IV). In some embodiments, the invention provides a composition comprising about 1-10% of a compound of structure (C-IV). In some embodiments, the invention provides a composition comprising about 1, 2, 5, 10, 20, 30, 40, 50, 60, 70, 80, 90, 95, 96, 97, 98, or 99% of a compound of structure (C-IV).

In some of these embodiments, the composition further comprises a pharmaceutically acceptable excipient.

In some embodiments, the invention provides a composition comprising at least about 1, 5, 10, 20, 30, 40, 50, 60, 70, 80, 90, 95, 99, 99.5, 99.9, or 99.99% of a compound of structure (C-V). In some embodiments, the invention provides a composition for the oral delivery of a compound of structure (C-V) comprising no more than about 2, 5, 10, 20, 30, 40, 50, 60, 70, 80, 90, 95, 99, 99.5, 99.9, 99.99, or 100% of a compound of structure (C-V). In some embodiments, the invention provides a composition comprising about 1-100% of a compound of structure (C-V), or about 10-100% of a compound of structure (C-V), or about 20-100% of a compound of structure (C-V), or about 50-100% of a compound of structure (C-V), or about 80-100% of a compound of structure (C-V), or about 90-100% of a compound of structure (C-V), or about 95-100% of a compound of structure (C-V), or about 99-100% of a compound of structure (C-V). In some embodiments, the invention provides a composition comprising about 1-90% of a compound of structure (C-V), or about 10-90% of a compound of structure (C-V), or about 20-90% of a compound of structure (C-V), or about 50-90 of a compound of structure (C-V), or about 80-90% of a compound of structure (C-V). In some embodiments, the invention provides a composition comprising about 1-75 of a compound of structure (C-V), or about 10-75% of a compound of structure (C-V), or about 20-75% of a compound of structure (C-V), or about 50-75% of a compound of structure (C-V). In some embodiments, the invention provides a composition comprising about 1-50% of a compound of structure (C-V), or about 10-50% of a compound of structure (C-V), or about 20-50% of a compound of structure (C-V), or about 30-50% of a compound of structure (C-V), or about 40-50% of a compound of structure (C-V). In some embodiments, the invention provides a composition comprising about 1-40% of a compound of structure (C-V), or about 10-40% of a compound of structure (C-V), or about 20-40% of a compound of structure (C-V), or about 30-40% of a compound of structure (C-V). In some embodiments, the invention provides a composition comprising about 1-30% of a compound of structure (C-V), or about 10-30% of a compound of structure (C-V), or about 20-30% of a compound of structure (C-V). In some embodiments, the invention provides a composition comprising about 1-20 of a compound of structure (C-V), or about 10-20% of a compound of structure (C-V). In some embodiments, the invention provides a composition comprising about 1-10% of a compound of structure (C-V). In some embodiments, the invention provides a composition comprising about 1, 2, 5, 10, 20, 30, 40, 50, 60, 70, 80, 90, 95, 96, 97, 98, or 99% of a compound of structure (C-V).

In some of these embodiments, the composition further comprises a pharmaceutically acceptable excipient.

In some embodiments, the invention provides a composition comprising at least about 1, 5, 10, 20, 30, 40, 50, 60, 70, 80, 90, 95, 99, 99.5, 99.9, or 99.99% of a compound of structure (C-VI). In some embodiments, the invention provides a composition for the oral delivery of a compound of structure (C-VI) comprising no more than about 2, 5, 10, 20, 30, 40, 50, 60, 70, 80, 90, 95, 99, 99.5, 99.9, 99.99, or 100% of a compound of structure (C-VI). In some embodiments, the invention provides a composition comprising about 1-100% of a compound of structure (C-VI), or about 10-100% of a compound of structure (C-VI), or about 20-100% of a compound of structure (C-VI), or about 50-100% of a compound of structure (C-VI), or about 80-100% of a compound of structure (C-VI), or about 90-100% of a compound of structure (C-VI), or about 95-100% of a compound of structure (C-VI), or about 99-100% of a compound of structure (C-VI). In some embodiments, the invention provides a composition comprising about 1-90% of a compound of structure (C-VI), or about 10-90% of a compound of structure (C-VI), or about 20-90% of a compound of structure (C-VI), or about 50-90 of a compound of structure (C-VI), or about 80-90% of a compound of structure (C-VI). In some embodiments, the invention provides a composition comprising about 1-75 of a compound of structure (C-VI), or about 10-75% of a compound of structure (C-VI), or about 20-75% of a compound of structure (C-VI), or about 50-75% of a compound of structure (C-VI). In some embodiments, the invention provides a composition comprising about 1-50% of a compound of structure (C-VI), or about 10-50% of a compound of structure (C-VI), or about 20-50% of a compound of structure (C-VI), or about 30-50% of a compound of structure (C-VI), or about 40-50% of a compound of structure (C-VI). In some embodiments, the invention provides a composition comprising about 1-40% of a compound of structure (C-VI), or about 10-40% of a compound of structure (C-VI), or about 20-40% of a compound of structure (C-VI), or about 30-40% of a compound of structure (C-VI). In some embodiments, the invention provides a composition comprising about 1-30% of a compound of structure (C-VI), or about 10-30% of a compound of structure (C-VI), or about 20-30% of a compound of structure (C-VI). In some embodiments, the invention provides a composition comprising about 1-20 of a compound of structure (C-VI), or about 10-20% of a compound of structure (C-VI). In some embodiments, the invention provides a composition comprising about 1-10% of a compound of structure (C-VI). In some embodiments, the invention provides a composition comprising about 1, 2, 5, 10, 20, 30, 40, 50, 60, 70, 80, 90, 95, 96, 97, 98, or 99% of a compound of structure (C-VI).

In some of these embodiments, the composition further comprises a pharmaceutically acceptable excipient.

In another aspect the invention provides compositions comprising a compound of structure (C-I) or a pharmaceutically acceptable salt thereof. In another aspect the invention provides compositions comprising a compound of structure (C-I) and a nucleoside or a nucleoside analog as described herein. In some embodiments, a compound of structure (C-I) is co-administered with a nucleoside or a nucleoside analog. In some embodiments the nucleoside or a nucleoside analog is a compound of structure (C-II), (C-III), (C-IV), (C-V) or (C-VI). In some embodiments the compositions further comprise a pharmaceutically acceptable excipient. In some embodiments, the compositions are administered orally. In some embodiments, the compositions are administered trandermally. In some embodiments, the compositions injected. Other forms of administration are also compatible with embodiments of the compositions of the invention, as described herein.

In some embodiments, the amount of the compound of structure (C-I) or a pharmaceutically acceptable salt thereof is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w.

In some embodiments, the amount of the compound of structure (C-II), (C-III), (C-IV), (C-V) or (C-VI) or pharmaceutically acceptable salts thereof is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w.

In some embodiments, the amount of the compound of structure (C-I) or pharmaceutically acceptable salts thereof is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w.

In some embodiments, the amount of the compound of structure (C-II), (C-III), (C-IV), (C-V) or (C-VI) or pharmaceutically acceptable salts thereof is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w.

In some embodiments, the amount of the compound of structure (C-I) or a pharmaceutically acceptable salt thereof is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w.

In some embodiments, the amount of the compound of structure (C-II), (C-III), (C-IV), (C-V) or (C-VI) or pharmaceutically acceptable salts thereof is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w.

In some embodiments, the amount of the compound of structure (C-I) or pharmaceutically acceptable salts thereof is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w.

In some embodiments, the amount of the compound of structure (C-II), (C-III), (C-IV), (C-V) or (C-VI) or pharmaceutically acceptable salts thereof is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w.

In some embodiments, the amount of the compound of structure (C-I) or pharmaceutically acceptable salts thereof is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the compound of structure (C-II), (C-III), (C-IV), (C-V) or (C-VI) or pharmaceutically acceptable salts thereof is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments the amount of the compound of structure (C-I) or pharmaceutically acceptable salts thereof is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments the amount of the compound of structure (C-II), (C-III), (C-IV), (C-V) or (C-VI) or pharmaceutically acceptable salts thereof is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the concentration of the compound of structure (C-I) or pharmaceutically acceptable salts thereof is in the range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments, the concentration of the compound of structure (C-II), (C-III), (C-IV), (C-V) or (C-VI) or pharmaceutically acceptable salts thereof is in the range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments, the invention relates to compositions comprising a compound of structure (C-I) and a compound of structure (C-II), where the compound of structure (C-I) is present in an amount from about 1-1000 mg, or about 10-1000 mg, or about 50-1000 mg, or about 100-1000 mg, or about 1-500 mg, or about 5-500 mg, or about 50-500 mg, or about 100-500 mg, or about 200-1000 mg, or about 200-800 mg, or about 400-800 mg, or about 400-800 mg, or about 1 mg, or about 10 mg, or about 25 mg, or about 50 mg, or about 100 mg, or about 200 mg, or about 250 mg, or about 300 mg, or about 400 mg, or about 500 mg, or about 600 mg, or about 700 mg, or about 800 mg, or about 900 mg, or about 1000 mg, and lamivudine is present in an amount from 0.01 to 1000 mg, or about 1-800 mg, or about 1-500 mg, or about 5-500 mg, or about 50-500 mg, or about 100-500 mg, or about 10, 20, 50, 80, 100, 150, 200, 300, 400, or 500 mg.

In some embodiments, the compound of structure (C-I) is present at about 100 mg and the compound of structure (C-II) is present at about 50 mg In some embodiments, the compound of structure (C-I) is present at about 600 mg and the compound of structure (C-II) is present at about 50 mg. In some embodiments, the compound of structure (C-I) is present at about 600 mg and the compound of structure (C-II) is present at about 150 mg. In some embodiments, the compound of structure (C-I) is present at about 600 mg and the compound of structure (C-II) is present at about 200 mg. In some embodiments, the compound of structure (C-I) is present at about 700 mg and the compound of structure (C-II) is present at about 50 mg. In some embodiments, the compound of structure (C-I) is present at about 700 mg and the compound of structure (C-II) is present at about 150 mg. In some embodiments, the compound of structure (C-I) is present at about 700 mg and the compound of structure (C-II) is present at about 200 mg. In some embodiments, the compound of structure (C-I) is present at about 800 mg and the compound of structure (C-II) is present at about 50 mg. In some embodiments, the compound of structure (C-I) is present at about 800 mg and the compound of structure (C-II) is present at about 150 mg. In some embodiments, the compound of structure (C-I) is present at about 800 mg and the compound of structure (C-II) is present at about 200 mg.

In other embodiments, compositions of the invention comprise the compound of structure (C-I) and the compound of structure (C-II), where the compound of structure (C-I) is present in an amount from about 1-1500 mg, or about 10-1500 mg, or about 50-1500 mg, or about 100-1500 mg, or about 500-1500 mg, or about 600-1500 mg, or about 700-1500 mg, or about 800-1500 mg, or about 200-1200 mg, or about 400-1200 mg, or about 600-1200 mg, or about 800-1200 mg, or about 100 mg, or about 200 mg, or about 300 mg, or about 500 mg, or about 600 mg, or about 700 mg, or about 800 mg, or about 900 mg, or about 1000 mg, or about 1100 mg, or about 1200 mg, or about 1500 mg, and the compound of structure (C-II) is present in an amount from 0.01 to 1000 mg, or about 1-800 mg, or about 1-500 mg, or about 1-250 mg, or about 1-100 mg, or about 1-50 mg, or about 1, 5, 10, 20, 50, 80, 100, 150, 200, 300, 400, or 500 mg.

In some embodiments, the compound of structure (C-I) is present at about 500 mg and the compound of structure (C-III) is present at about 5 mg. In some embodiments, the compound of structure (C-I) is present at about 800 mg and the compound of structure (C-III) is present at about 5 mg. In some embodiments, the compound of structure (C-I) is present at about 800 mg and the compound of structure (C-III) is present at about 15 mg. In some embodiments, the compound of structure (C-I) is present at about 800 mg and the compound of structure (C-III) is present at about 20 mg. In some embodiments, the compound of structure (C-I) is present at about 1000 mg and the compound of structure (C-III) is present at about 5 mg. In some embodiments, the compound of structure (C-I) is present at about 1000 mg and the compound of structure (C-III) is present at about 15 mg. In some embodiments, the compound of structure (C-I) is present at about 1000 mg and the compound of structure (C-III) is present at about 20 mg. In some embodiments, the compound of structure (C-I) is present at about 1200 mg and the compound of structure (C-III) is present at about 5 mg. In some embodiments, the compound of structure (C-I) is present at about 1200 mg and the compound of structure (C-III) is present at about 15 mg. In some embodiments, the compound of structure (C-I) is present at about 1200 mg and the compound of structure (C-III) is present at about 20 mg.

In some embodiments, compositions of the invention include the compound of structure (C-I) and the compound of structure (C-IV), where the compound of structure (C-I) is present in an amount from about 1-1500 mg, or about 10-1500 mg, or about 50-1500 mg, or about 100-1500 mg, or about 500-1500 mg, or about 600-1500 mg, or about 700-1500 mg, or about 800-1500 mg, or about 200-1200 mg, or about 400-1200 mg, or about 600-1200 mg, or about 800-1200 mg, or about 600-800 mg, or about 100 mg, or about 200 mg, or about 300 mg, or about 500 mg, or about 600 mg, or about 700 mg, or about 800 mg, or about 900 mg, or about 1000 mg, or about 1100 mg, or about 1200 mg, or about 1500 mg, and the compound of structure (C-IV) is present in an amount from 1 to 1500 mg, or about 10-1500 mg, or about 50-1500 mg, or about 100-1500 mg, or about 500-1500 mg, or about 600-1500 mg, or about 700-1500 mg, or about 800-1500 mg, or about 200-1200 mg, or about 400-1200 mg, or about 600-1200 mg, or about 800-1200 mg, or about 600-800 mg, or about 100 mg, or about 200 mg, or about 300 mg, or about 500 mg, or about 600 mg, or about 700 mg, or about 800 mg, or about 900 mg, or about 1000 mg, or about 1100 mg, or about 1200 mg, or about 1500 mg.

In some embodiments, the compound of structure (C-I) is present at about 500 mg and the compound of structure (C-IV) is present at about 500 mg. In some embodiments, the compound of structure (C-I) is present at about 600 mg and the compound of structure (C-IV) is present at about 600 mg. In some embodiments, the compound of structure (C-I) is present at about 600 mg and the compound of structure (C-IV) is present at about 800 mg. In some embodiments, the compound of structure (C-I) is present at about 600 mg and the compound of structure (C-IV) is present at about 1200 mg. In some embodiments, the compound of structure (C-I) is present at about 800 mg and the compound of structure (C-IV) is present at about 600 mg. In some embodiments, the compound of structure (C-I) is present at about 800 mg and the compound of structure (C-IV) is present at about 800 mg. In some embodiments, the compound of structure (C-I) is present at about 800 mg and the compound of structure (C-IV) is present at about 1200 mg. In some embodiments, the compound of structure (C-I) is present at about 1200 mg and the compound of structure (C-IV) is present at about 600 mg. In some embodiments, the compound of structure (C-I) is present at about 1200 mg and the compound of structure (C-IV) is present at about 800 mg. In some embodiments, the compound of structure (C-I) is present at about 1200 mg and the compound of structure (C-IV) is present at about 1200 mg.

In some embodiments, compositions of the invention include the compound of structure (C-I) and the compound of structure (C-V), where the compound of structure (C-I) is present in an amount from about 1-1000 mg, or about 10-1000 mg, or about 50-1000 mg, or about 100-1000 mg, or about 1-500 mg, or about 5-500 mg, or about 50-500 mg, or about 100-500 mg, or about 200-1000 mg, or about 200-800 mg, or about 400-800 mg, or about 400-800 mg, or about 1 mg, or about 10 mg, or about 25 mg, or about 50 mg, or about 100 mg, or about 200 mg, or about 250 mg, or about 300 mg, or about 400 mg, or about 500 mg, or about 600 mg, or about 700 mg, or about 800 mg, or about 900 mg, or about 1000 mg, and the compound of structure (C-V) is present in an amount from 0.01 to 1000 mg, or about 1-800 mg, or about 1-500 mg, or about 5-500 mg, or about 50-500 mg, or about 100-500 mg, or about 10, 20, 50, 80, 100, 150, 200, 300, 400, or 500 mg.

In some embodiments, the compound of structure (C-I) is present at about 100 mg and the compound of structure (C-V) is present at about 50 mg. In some embodiments, the compound of structure (C-I) is present at about 600 mg and the compound of structure (C-V) is present at about 200 mg. In some embodiments, the compound of structure (C-I) is present at about 600 mg and the compound of structure (C-V) is present at about 300 mg. In some embodiments, the compound of structure (C-I) is present at about 600 mg and the compound of structure (C-V) is present at about 400 mg. In some embodiments, the compound of structure (C-I) is present at about 700 mg and the compound of structure (C-V) is present at about 200 mg. In some embodiments, the compound of structure (C-I) is present at about 700 mg and the compound of structure (C-V) is present at about 300 mg. In some embodiments, the compound of structure (C-I) is present at about 700 mg and the compound of structure (C-V) is present at about 400 mg. In some embodiments, the compound of structure (C-I) is present at about 800 mg and the compound of structure (C-V) is present at about 200 mg. In some embodiments, the compound of structure (C-I) is present at about 800 mg and the compound of structure (C-V) is present at about 300 mg. In some embodiments, the compound of structure (C-I) is present at about 800 mg and the compound of structure (C-V) is present at about 400 mg.

In other embodiments, compositions of the invention include the compound of structure (C-I) and the compound of structure (C-VI), where the compound of structure (C-I) is present in an amount from about 1-1500 mg, or about 10-1500 mg, or about 50-1500 mg, or about 100-1500 mg, or about 500-1500 mg, or about 600-1500 mg, or about 700-1500 mg, or about 800-1500 mg, or about 200-1200 mg, or about 400-1200 mg, or about 600-1200 mg, or about 800-1200 mg, or about 100 mg, or about 200 mg, or about 300 mg, or about 500 mg, or about 600 mg, or about 700 mg, or about 800 mg, or about 900 mg, or about 1000 mg, or about 1100 mg, or about 1200 mg, or about 1500 mg, and the compound of structure (C-VI) is present in an amount from 0.01 to 1000 mg, or about 1-800 mg, or about 1-600 mg, or about 100-600 mg, or about 200-600 mg, or about 1, 5, 10, 20, 50, 80, 100, 150, 200, 300, 400, 500, 600, 700 or 800 mg.

In some embodiments, the compound of structure (C-I) is present at about 500 mg and the compound of structure (C-VI) is present at about 100 mg. In some embodiments, the compound of structure (C-I) is present at about 800 mg and the compound of structure (C-VI) is present at about 200 mg. In some embodiments, the compound of structure (C-I) is present at about 800 mg and the compound of structure (C-VI) is present at about 400 mg. In some embodiments, the compound of structure (C-I) is present at about 800 mg and the compound of structure (C-VI) is present at about 600 mg. In some embodiments, the compound of structure (C-I) is present at about 1000 mg and the compound of structure (C-VI) is present at about 200 mg. In some embodiments, the compound of structure (C-I) is present at about 1000 mg and the compound of structure (C-VI) is present at about 400 mg. In some embodiments, the compound of structure (C-I) is present at about 1000 mg and the compound of structure (C-VI) is present at about 600 mg. In some embodiments, the compound of structure (C-I) is present at about 1200 mg and the compound of structure (C-VI) is present at about 200 mg. In some embodiments, the compound of structure (C-I) is present at about 1200 mg and the compound of structure (C-VI) is present at about 400 mg. In some embodiments, the compound of structure (C-I) is present at about 1200 mg and the compound of structure (C-VI) is present at about 600 mg.

In some embodiments, compositions of the invention include the compound of structure (C-I) and emtricitabine, where the compound of structure (C-I) is present in an amount from about 1-1500 mg, or about 10-1500 mg, or about 50-1500 mg, or about 100-1500 mg, or about 500-1500 mg, or about 600-1500 mg, or about 700-1500 mg, or about 800-1500 mg, or about 200-1200 mg, or about 400-1200 mg, or about 600-1200 mg, or about 800-1200 mg, or about 100 mg, or about 200 mg, or about 300 mg, or about 500 mg, or about 600 mg, or about 700 mg, or about 800 mg, or about 900 mg, or about 1000 mg, or about 1100 mg, or about 1200 mg, or about 1500 mg, and emtricitabine is present in an amount from 0.01 to 1000 mg, or about 1-800 mg, or about 1-500 mg, or about 5-500 mg, or about 50-500 mg, or about 100-500 mg, or about 10, 20, 50, 80, 100, 150, 200, 300, 400, or 500 mg.

In some embodiments, the molar ratio of the compound of structure (C-I) to the compound of structure (C-II), (C-III), (IV0, (C-V) or (C-VI) is 0.0001:1 to 1:1, about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1.

In some embodiments, the molar ratio of the compound of structure (C-I) to the compound of structure (C-II), (C-III), (IV0, (C-V) or (C-VI) is about 0.03×10⁻⁵:1, 0.1×10⁻⁵:1, 0.04×10⁻³:1, 0.03×10⁻⁵:1, 0.02×10⁻⁵:1, 0.01×10⁻³:1, 0.1×10⁻³:1, 0.15×10⁻³:1, 0.2×10⁻³:1, 0.3×10⁻³:1, 0.4×10⁻³:1, 0.5×10⁻³:1, 0.15×10⁻²:1, 0.1×10⁻²:1, 0.2×10⁻²:1, 0.3×10⁻²:1, 0.4×10⁻²:1, 0.5×10⁻²:1, 0.6×10⁻²:1, 0.8×10⁻²:1, 0.01:1, 0.1:1; or 0.2:1 per dose.

In some embodiments, the molar ratio of the compound of structure (C-I) to the compound of structure (C-II), (C-III), (IV0, (C-V) or (C-VI) is about 0.001:1, 0.002:1, 0.003:1, 0.004:1, 0.005:1, 0.006:1, 0.007:1, 0.008:1, 0.009:1, 0.01:1, 0.02:1, 0.03:1, 0.04:1, 0.05:1, 0.06:1, 0.07:1, 0.08:1, 0.09:1, 0.1:1, 0.2:1, 0.3:1, 0.4:1, 0.5:1, 0.6:1, 0.7:1, 0.8:1, 0.9:1, 1:1, 2:1, 3:1, 4:1, or 5:1 per dose.

### Pharmaceutical Compositions

This invention provides pharmaceutical compositions that contain, as the active ingredient, a compound of general formula (A-I) as described herein, or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants.

This invention further provides pharmaceutical compositions comprising, a compound of general formula (A-I) or a pharmaceutically acceptable salt thereof, another agent as described herein, or a pharmaceutically acceptable salts thereof, and one or more pharmaceutically acceptable excipients, carriers, including inert solid diluents and fillers, diluents, including sterile aqueous solution and various organic solvents, permeation enhancers, solubilizers and adjuvants.

This invention provides pharmaceutical compositions that contain, a compound of general formula (B-I) or (B-I-A) as described herein, or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants.

This invention further provides pharmaceutical compositions that contain, a compound of general formula (B-I) or (B-I-A) or a pharmaceutically acceptable salt thereof, another agent as described herein, or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants.

This invention provides pharmaceutical compositions comprising, a compound of structure (C-I), or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants.

This invention further provides pharmaceutical compositions comprising, a compound of structure (C-I), or a pharmaceutically acceptable salt thereof, and a compound of structure (C-II). (C-III), (C-IV), (C-V) or (C-VI), or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants.

The composition described herein may be prepared as pharmaceutical compositions in dosages as described herein (see, e.g., Compositions). Such compositions are prepared in a manner well known in the pharmaceutical art.

### Pharmaceutical Compositions Useful for Oral Administration.

In some embodiments, the invention provides a pharmaceutical composition useful for oral administration comprising a compound of formula (A-I), or a pharmaceutically acceptable salt thereof, and a pharmaceutical excipient for oral administration.

In some embodiments, the invention provides a solid pharmaceutical composition useful for oral administration comprising: i) an effective amount of a compound of general formula (A-I), or a pharmaceutically acceptable salt thereof, and ii) a pharmaceutical excipient for oral administration.

In some embodiments, the composition further comprises: iii) an effective amount of a second agent. Examples of agents that can be used in combination with compounds of general formula (A-I) include but are not limited to nucleosides, nucleosides analogs, interferons such as □, □ or □-interferon, renal excretion inhibitors such as probenecid, nucleoside transport inhibitors such as dipyridamole, immunomodulators such as interleukin II (IL2) and granulocyte macrophage colony stimulating factor (GM-CSF), erythropoetin, empligen, thymomudulin, thymopentin, foscarnet, ribavirin and inhibitors of HIV binding to CD4 receptors e.g. soluble CD4, CD4 fragments, CD4 hybrid molecules, glycosylation inhibitors such as 2-deoxy-D-glucose, castanospermine and 1-deoxynojirimycin.

In some embodiments, the pharmaceutical composition may be a liquid pharmaceutical composition for oral consumption. In some embodiments, the other agent is lamivudine, racivir, elvucitabine, apricitabine or emtricitabine.

In some embodiments, the invention provides a solid pharmaceutical composition useful for oral administration containing an effective amount of a compound of general formula (A-I), and a pharmaceutically acceptable excipient. In some embodiments, the invention provides a liquid pharmaceutical composition for oral administration containing an effective amount of a compound of formula (A-I), and a pharmaceutically acceptable excipient.

In some embodiments, the invention provides a pharmaceutical composition useful or oral administration comprising a compound of formula (B-I) or (B-I-A), or a pharmaceutically acceptable salt thereof, and a pharmaceutical excipient for oral administration.

In some embodiments, the invention provides a solid pharmaceutical composition for oral administration containing: (i) an effective amount of a compound of general formula (B-I) or (B-I-A), or pharmaceutically acceptable salts thereof, and (ii) a pharmaceutical excipient for oral administration.

In some embodiments, the composition further comprises: iii) an effective amount of a second agent. Examples of agents that can be used in combination with compounds of general formula (B-I) or (B-I-A) include but are not limited to nucleosides, nucleosides analogs, interferons such as □, □ or □-interferon, renal excretion inhibitors such as probenecid, nucleoside transport inhibitors such as dipyridamole, immunomodulators such as interleukin II (IL2) and granulocyte macrophage colony stimulating factor (GM-CSF), erythropoetin, empligen, thymomudulin, thymopentin, foscarnet, ribavirin and inhibitors of HIV binding to CD4 receptors e.g. soluble CD4, CD4 fragments, CD4 hybrid molecules, glycosylation inhibitors such as 2-deoxy-D-glucose, castanospermine and 1-deoxynojirimycin.

In some embodiments, the pharmaceutical composition may be a liquid pharmaceutical composition useful for oral administration. In some embodiments, the other agent is lamivudine, racivir, elvucitabine, apricitabine or emtricitabine.

In some embodiments, the invention provides a solid pharmaceutical composition administration containing an effective amount of a compound of general formula (B-I) or (B-I-A), and a pharmaceutically acceptable excipient. In some embodiments, the invention provides a liquid pharmaceutical composition useful for oral administration containing an effective amount of a compound of formula (B-I) or (B-I-A), and a pharmaceutically acceptable excipient.

In some embodiments, the invention provides a pharmaceutical composition useful for oral administration comprising a compound of structure (C-I) and a compound of structure (C-II), (C-III), (C-IV), (C-V) or (C-VI), or a pharmaceutically acceptable salt thereof, and a pharmaceutical excipient for oral administration.

In some embodiments, the composition further contains an effective amount of a third agent.

In some embodiments, the pharmaceutical composition may be a liquid pharmaceutical composition for oral consumption.

In some embodiments, the invention provides a solid pharmaceutical composition useful for oral administration containing an effective amount of a compound of structure (C-I), an effective amount of a compound of structure (C-II), and a pharmaceutically acceptable excipient. In some embodiments, the invention provides a liquid pharmaceutical composition useful for oral administration containing an effective amount of a compound of structure (C-I), an effective amount of a compound of structure (C-II), and a pharmaceutically acceptable excipient.

In some embodiments, the invention provides a solid pharmaceutical composition useful for oral administration comprising a compound of structure (C-I) at about 100-800 mg, a compound of structure (C-II) at about 10-200 mg and a pharmaceutically acceptable excipient. In some embodiments, the invention provides a liquid pharmaceutical composition useful for oral administration containing a compound of structure (C-I) at about 0.1-800 mg/ml, a compound of structure (C-II) at about 0.05-200 mg/ml and a pharmaceutically acceptable excipient.

In some embodiments, the invention provides a solid pharmaceutical composition useful for oral administration containing an effective amount of a compound of structure (C-I), an effective amount of compound of structure (C-III), and a pharmaceutically acceptable excipient. In some embodiments, the invention provides a liquid pharmaceutical composition useful for oral administration containing an effective amount of a compound of structure (C-I), an effective amount of compound of structure (C-III), and a pharmaceutically acceptable excipient.

In some embodiments, the invention provides a solid pharmaceutical composition useful for oral administration comprising a compound of structure (C-I) at about 100-1200 mg, compound of structure (C-III) at about 10-200 mg and a pharmaceutically acceptable excipient. In some embodiments, the invention provides a liquid pharmaceutical composition useful for oral administration comprising a compound of structure (C-I) at about 0.1-1200 mg/ml, compound of structure (C-III) at about 0.05-200 mg/ml and a pharmaceutically acceptable excipient.

In some embodiments, the invention provides a solid pharmaceutical composition useful for oral administration containing an effective amount of a compound of structure (C-I), an effective amount of a compound of structure (C-IV), and a pharmaceutically acceptable excipient. In some embodiments, the invention provides a liquid pharmaceutical composition useful for oral administration containing an effective amount of a compound of structure (C-I), an effective amount of a compound of structure (C-IV), and a pharmaceutically acceptable excipient.

In some embodiments, the invention provides a solid pharmaceutical composition useful for oral administration containing a compound of structure (C-I) at about 100-1200 mg, a compound of structure (C-IV) at about 100-1200 mg and a pharmaceutically acceptable excipient. In some embodiments, the invention provides a liquid pharmaceutical composition useful for oral administration containing a compound of structure (C-I) at about 0.1-1200 mg/ml, a compound of structure (C-IV) at about 0.05-1200 mg/ml and a pharmaceutically acceptable excipient.

In some embodiments, the invention provides a solid pharmaceutical composition useful for oral administration containing an effective amount of a compound of structure (C-I), an effective amount of a compound of structure (C-V), and a pharmaceutically acceptable excipient. In some embodiments, the invention provides a liquid pharmaceutical composition useful for oral administration containing an effective amount of a compound of structure (C-I), an effective amount of a compound of structure (C-V), and a pharmaceutically acceptable excipient.

In some embodiments, the invention provides a solid pharmaceutical composition useful for oral administration containing a compound of structure (C-I) at about 100-1200 mg, a compound of structure (C-V) at about 100-1200 mg and a pharmaceutically acceptable excipient. In some embodiments, the invention provides a liquid pharmaceutical composition useful for oral administration containing a compound of structure (C-I) at about 0.1-1200 mg/ml, a compound of structure (C-V) at about 0.05-1200 mg/ml and a pharmaceutically acceptable excipient.

In some embodiments, the invention provides a solid pharmaceutical composition useful for oral administration containing an effective amount of a compound of structure (C-I), an effective amount of a compound of structure (C-VI), and a pharmaceutically acceptable excipient. In some embodiments, the invention provides a liquid pharmaceutical composition useful for oral administration containing an effective amount of a compound of structure (C-I), an effective amount of a compound of structure (C-VI), and a pharmaceutically acceptable excipient.

In some embodiments, the invention provides a solid pharmaceutical composition useful for oral administration containing a compound of structure (C-I) at about 100-1200 mg, a compound of structure (C-VI) at about 10-800 mg and a pharmaceutically acceptable excipient. In some embodiments, the invention provides a liquid pharmaceutical composition useful for oral administration containing a compound of structure (C-I) at about 0.1-1200 mg/ml, a compound of structure (C-VI) at about 0.05-800 mg/ml and a pharmaceutically acceptable excipient.

Pharmaceutical compositions of the invention which may be used for oral administration may be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by one of skill in the art. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a machine a mixture of the powdered compound moistened with an inert liquid diluent.

This invention further encompasses anhydrous pharmaceutical compositions and dosage forms comprising an active ingredient, since water can facilitate the degradation of some compounds. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in formulary kits. Examples of packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, carriers include powders, capsules, and tablets, with the solid oral preparations. If desired, tablets can be coated by standard aqueous or nonaqueous techniques.

Binders for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the compositions described herein may further comprise various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A hydrophilic surfactant may generally have an HLB value of at least 10, while lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10.

However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-100 leate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. In some embodiments, the lipophilic surfactants are glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to help provide good solubilization and to minimize precipitation. This can be particularly useful for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Solubilizers include sorbitol, glycerol, triacetin, ethyl alcohol, PEG-400, glycofurol and propylene glycol.

The amount of solubilizer that can be included is not particularly limited. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, disopropylethylamine, ethanolamine, ethylenediamine, triethanolamine, triethylamine, trisopropanolamine, trimethylamine, tris(hydroxymethyl)aminomethane (TRIS) and the like. Bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of inorganic acids include hydrochloric acid, hydrobromic acid, hydriodic acid, sulfuric acid, nitric acid, boric acid, phosphoric acid, and the like. Examples of organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions Useful for Injection

In some embodiments, the invention provides a pharmaceutical composition for injection comprising a compound of formula (A-I) or a pharmaceutically acceptable salt thereof, and a pharmaceutical excipient for injection. In some embodiments, the invention provides a pharmaceutical composition for injection containing a combination of a compound of formula (A-I) or a pharmaceutically acceptable salt thereof, another agent and a pharmaceutical excipient for injection. Examples of components and amounts of agents in the compositions are described herein.

In some embodiments, the invention provides a pharmaceutical composition for injection comprising a compound of formula (B-I) or (B-I-A) or a pharmaceutically acceptable salt thereof, and a pharmaceutical excipient for injection. In some embodiments, the invention provides a pharmaceutical composition for injection containing a compound of formula (B-I) or (B-I-A) or a pharmaceutically acceptable salt thereof, another agent and a pharmaceutical excipient for injection. Examples of components and amounts of agents in the compositions are described herein.

In some embodiments, the invention provides a pharmaceutical composition for injection comprising a compound of structure (C-I) or a pharmaceutically acceptable salt thereof, a compound of structure (C-II), (C-III), (C-IV) (C-V) or (C-VI), and a pharmaceutical excipient for injection. In some embodiments, the invention provides a pharmaceutical composition for injection comprising a compound of structure (C-I) or a pharmaceutically acceptable salt thereof, a compound of structure (C-II), (C-III), (C-IV) (C-V) or (C-VI), another agent and a pharmaceutical excipient for injection. Examples of components and amounts of agents in the compositions are described herein.

The forms in which the compositions described herein may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the appropriate compound in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, methods of preparation include vacuum-drying and freeze-drying techniques which yield a powder of the active ingredient plus any additional desired ingredient from a previously sterile-filtered solution thereof.

Pharmaceutical Compositions Useful for Topical (e.g. Transdermal) Delivery

In some embodiments, the invention provides a pharmaceutical composition for transdermal delivery comprising a compound of formula (A-I), or a pharmaceutically acceptable salt thereof, and a pharmaceutical excipient for transdermal delivery. In some embodiments, the invention provides a composition comprising a compound of formula (A-I) or a pharmaceutically acceptable salt thereof, another agent, and a pharmaceutical excipient for transdermal delivery.

In some embodiments, the invention provides a pharmaceutical composition for transdermal delivery comprising a compound of formula (B-I) or (B-I-A), or a pharmaceutically acceptable salt thereof, and a pharmaceutical excipient for transdermal delivery. In some embodiments, the invention provides a composition comprising a compound of formula (B-I) or (B-I-A) or a pharmaceutically acceptable salt thereof, another agent, and a pharmaceutical excipient for transdermal delivery.

In some embodiments, the invention provides a pharmaceutical composition for transdermal delivery comprising a compound of structure (C-I) and a compound of structure (C-II), (C-III), (C-IV) (C-V) or (C-VI), and a pharmaceutical excipient for transdermal delivery. In some embodiments, the invention provides a pharmaceutical composition for transdermal delivery comprising a compound of structure (C-I) and a compound of structure (C-II), (C-III), (C-IV) (C-V) or (C-VI), another agent and a pharmaceutical excipient for transdermal delivery. Components and amounts of the components in the compositions are as described herein.

Compositions of the present invention can be formulated into preparations in solid, semi-solid, or liquid forms for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise solid or gel phase carriers or excipients, which are compounds that allow increased penetration of, or assist in the delivery of, therapeutic molecules across the stratum corneum permeability barrier of the skin. There are many of these penetration-enhancing molecules known to those skilled in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another formulation for use in the methods of the present invention employs transdermal delivery devices (e.g. patches or minipumps).

Such transdermal devices may be used to provide continuous or discontinuous infusion of the compounds of general formula (A-I), in controlled amounts, either with or without a nucleoside or nucleoside analog. Thus, in some embodiments the invention provides a transdermal device incorporating a compound of general formula (A-I). In some embodiments the invention provides a transdermal device incorporating a compound of general formula (A-I) in combination with another agent such a nucleoside or nucleoside analog, e.g. lamivudine.

Such transdermal devices may be used to provide continuous or discontinuous infusion of the compounds of general formula (B-I) or (B-I-A), in controlled amounts, either with or without a nucleoside or nucleoside analog. Thus, in some embodiments the invention provides a transdermal device incorporating a compound of general formula (B-I) or (B-I-A). In some embodiments the invention provides a transdermal device incorporating a compound of general formula (B-I) or (B-I-A) in combination with another agent such as a nucleoside or nucleoside analog, e.g. lamivudine.

Such transdermal devices may be used to provide continuous or discontinuous infusion of the compound of formula (C-I), in controlled amounts, either with or without a compound of formula (C-II), (C-III), (C-IV), (C-V) or (C-VI). Thus, in some embodiments the invention provides a transdermal device comprising a compound of structure (C-I). In some embodiments the invention provides a transdermal device comprising a compound of structure (C-I) and a compound of structure (C-II).

The construction and use of transdermal devices for the delivery of pharmaceutical agents is well known in the art. See, e.g., U.S. Pat. Nos. 5,023,252, 4,992,445 and 5,001,139. Such devices may be constructed for continuous, pulsatile, or on demand delivery of pharmaceutical agents.

### Pharmaceutical Compositions Useful for Inhalation

Compositions for inhalation or insufflation include solutions and suspensions in pharmaceutically acceptable, aqueous or organic solvents, or mixtures thereof, and powders. The liquid or solid compositions may contain pharmaceutically acceptable excipients as described supra. The compositions can be administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, orally or nasally, from devices that deliver the formulation in an appropriate manner.

### Other Pharmaceutical Compositions

Pharmaceutical compositions may also be prepared from compositions described herein and one or more pharmaceutically acceptable excipients for sublingual, buccal, rectal, intraosseous, intraocular, intranasal, epidural, or intraspinal administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

### Diseases

The compounds and compositions described herein display valuable pharmacological properties.

In some embodiments, the compounds and compositions described herein are useful for the treatment or prophylaxis of a viral infection.

In some embodiments, the compounds and compositions described herein are useful for the treatment or prophylaxis of infections caused by DNA viruses, such as e.g. herpes simplex virus, the cytomegalovirus, Papovavirus, the varicella zoster virus or Epstein-Barr virus.

In some embodiments, the compounds and compositions described herein are useful for the treatment or prophylaxis of infections caused by RNA viruses, such as togaviruses or retroviruses.

In some embodiments, the compounds and compositions described herein are useful for the treatment or prophylaxis of infections caused by oncoviruses.

In some embodiments, the compounds and compositions described herein are useful for the treatment or prophylaxis of infections caused by HTLV-I.

In some embodiments, the compounds and compositions described herein are useful for the treatment or prophylaxis of infections caused by HTLV-II.

In some embodiments, the compounds and compositions described herein are useful for the treatment or prophylaxis of infections which are caused by lentiviruses.

In some embodiments, the compounds and compositions described herein are useful for the treatment or prophylaxis of the clinical manifestations of retroviral HIV infection in humans, such as persistent generalized lymphadenopathy (PGL), the advanced state of AIDS-related complex (ARC) and the clinically complete picture of AIDS.

In some embodiments, the compounds and compositions described herein are useful for the treatment or prophylaxis of infections caused by HIV-1.

In some embodiments, the compounds and compositions described herein are useful for the treatment or prophylaxis of infections caused by HIV-2.

In some embodiments, the compounds and compositions described herein are useful for the treatment or prophylaxis of chronic hepatitis B.

In another aspect the invention is directed to methods of treating cancer comprising administering to a subject in need thereof an effective amount of a compound or composition as described herein.

In some embodiments, the cancer is multiple myeloma, leukemia, lymphoma, acute leukemia, chronic leukemia, acute lymphocytic leukemia (ALL), acute nonlymphocytic leukemia (ANLL), chronic lymphocytic leukemia (CLL), chronic myelogenous leukemia (CML), acute myeloid leukemia (AML), hairy cell leukemia, Hodgkin's lymphoma, non-Hodgkin's lymphoma, multiple myeloma, hematologic cancer is of low, intermediate, or high grade, brain cancer, cancers of the head and neck, lung cancer, breast cancer, cancers of the reproductive system, cancers of the digestive system, pancreatic cancer, and cancers of the urinary system, cancer of the upper digestive tract or colorectal cancer, bladder cancer, renal cell carcinoma, prostate cancer, cancers of oral cavity and pharynx, cancers of the respiratory system, cancers of bones and joints, cancers of soft tissue, skin cancers, cancers of the genital system, cancers of the eye and orbit, cancers of the nervous system, cancers of the lymphatic system, and cancers of the endocrine system. In certain embodiments, these cancer s may be selected from the group consisting of: cancer of the tongue, mouth, pharynx, or other oral cavity; esophageal cancer, stomach cancer, or cancer of the small intestine; colon cancer or rectal, anal, or anorectal cancer; cancer of the liver, intrahepatic bile duct, gallbladder, pancreas, or other biliary or digestive organs; laryngeal, bronchial, and other cancers of the respiratory organs; heart cancer, melanoma, basal cell carcinoma, squamous cell carcinoma, other non-epithelial skin cancer; uterine or cervical cancer; uterine corpus cancer; ovarian, vulvar, vaginal, or other female genital cancer; prostate, testicular, penile or other male genital cancer; urinary bladder cancer; cancer of the kidney; renal, pelvic, or urethral cancer or other cancer of the genito-urinary organs; thyroid cancer or other endocrine cancer; and cutaneous T-cell lymphoma, both granulocytic and monocytic, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, hemangioblastoma, hepatocellular carcinoma, hepatoma, Kaposi's sarcoma, large cell carcinoma, leiomyosarcoma, liposarcoma, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, Wilm's tumor, ductal carcinoma in duct tissue in a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarinoma that has migrated to the bone; pancreatic cancer such as epitheliod carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, small cell cancers; myelodysplasia, myeloproliferative disorders; bone cancer; lung cancer such as non-small cell lung cancer (NSCLC), squamous cell carcinomas, adenocarcinomas, large cell undifferentiated carcinomas, small cell lung cancer; skin cancer, basal cell carcinoma, melanoma, squamous cell carcinoma actinic keratosis, eye retinoblastoma; cutaneous or intraocular (eye) melanoma; primary liver cancer (cancer that begins in the liver); kidney cancer; thyroid cancer such as papillary, follicular, medullary and anaplastic; AIDS-related lymphoma such as diffuse large B-cell lymphoma, B-cell immunoblastic lymphoma and small non-cleaved cell lymphoma; Kaposi's Sarcoma; viral-induced cancers including hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer; central nervous system cancers (CNS), primary brain tumors, gliomas (astrocytoma, anaplastic astrocytoma, or glioblastoma multiforme), Oligodendroglioma, Ependymoma, Meningioma, Lymphoma, Schwannoma, Medulloblastoma; peripheral nervous system (PNS) cancers, acoustic neuromas, malignant peripheral nerve sheath tumor (MPNST) including neurofibromas and schwannomas, malignant fibrous cytoma, malignant fibrous histiocytoma, malignant meningioma, malignant mesothelioma, and malignant mixed Müllerian tumor; oral cavity and oropharyngeal cancer such as, hypopharyngeal cancer, laryngeal cancer, nasopharyngeal cancer, and oropharyngeal cancer; stomach cancer such as lymphomas, gastric stromal tumors, and carcinoid tumors; testicular cancer such as germ cell tumors (GCTs), which include seminomas and nonseminomas, and gonadal stromal tumors, which include Leydig cell tumors and Sertoli cell tumors; thymus cancer such as to thymomas, thymic carcinomas, carcinoids or carcinoid tumors; rectal cancer; and colon cancer.

For the treatment of oncologic diseases and cancers, the compounds and compositions described herein may be administered with an agent selected from aromatase inhibitors, antiestrogen, anti-androgen, corticosteroids, gonadorelin agonists, topoisomerase 1 and 2 inhibitors, microtubule active agents, alkylating agents, nitrosoureas, antineoplastic antimetabolites, platinum containing compounds, lipid or protein kinase targeting agents, IMiDs, protein or lipid phosphatase targeting agents, anti-angiogenic agents, Akt inhibitors, IGF-I inhibitors, FGF3 modulators, mTOR inhibitors, Smac mimetics, HDAC inhibitors, agents that induce cell differentiation, bradykinin 1 receptor antagonists, angiotensin II antagonists, cyclooxygenase inhibitors, heparanase inhibitors, lymphokine inhibitors, cytokine inhibitors, IKK inhibitors, P38MAPK inhibitors, ARRY-797, HSP90 inhibitors, multlikinase inhibitors, bisphosphanates, rapamycin derivatives, anti-apoptotic pathway inhibitors, apoptotic pathway agonists, PPAR agonists, RAR agonists, inhibitors of Ras isoforms, telomerase inhibitors, protease inhibitors, metalloproteinase inhibitors, aminopeptidase inhibitors, SHIP activators-AQX-MN100, Humax-CD20 (ofatumumab), CD20 antagonists, IL2-diptheria toxin fusions, dacarbazine (DTIC), actinomycins C₂, C₃, D, and F₁, cyclophosphamide, melphalan, estramustine, maytansinol, rifamycin, streptovaricin, doxorubicin, daunorubicin, epirubicin, idarubicin, detorubicin, caminomycin, idarubicin, epirubicin, esorubicin, mitoxantrone, bleomycins A, A₂, and B, camptothecin, Irinotecan.RTM., Topotecan.RTM., 9-aminocamptothecin, 10,11-methylenedioxycamptothecin, 9-nitrocamptothecin, bortezomib, temozolomide, TAS103, NPI0052, combretastatin, combretastatin A-2, combretastatin A-4, calicheamicins, neocarcinostatins, epothilones A B, C, and semi-synthetic variants, Herceptin.RTM., Rituxan.RTM., CD40 antibodies, asparaginase, interleukins, interferons, leuprolide, and pegaspargase, 5-fluorouracil, fluorodeoxyuridine, ptorafur, 5′-deoxyfluorouridine, UFT, MITC, S-1 capecitabine, diethylstilbestrol, tamoxifen, toremefine, tolmudex, thymitaq, flutamide, fluoxymesterone, bicalutamide, finasteride, estradiol, trioxifene, dexamethasone, leuproelin acetate, estramustine, droloxifene, medroxyprogesterone, megesterol acetate, aminoglutethimide, testolactone, testosterone, diethylstilbestrol, hydroxyprogesterone, mitomycins A, B and C, porfiromycin, cisplatin, carboplatin, oxaliplatin, tetraplatin, platinum-DACH, ormaplatin, thalidomide, lenalidomide, CI-973, telomestatin, CHIR258, Rad 001, SAHA, Tubacin, 17-AAG, sorafenib, JM-216, podophyllotoxin, epipodophyllotoxin, etoposide, teniposide, Tarceva.RTM., Iressa.RTM., Imatinib.RTM., Miltefosine.RTM., Perifosine.RTM., aminopterin, methotrexate, methopterin, dichloro-methotrexate, 6-mercaptopurine, thioguanine, azattuoprine, allopurinol, cladribine, fludarabine, pentostatin, 2-chloroadenosine, deoxycytidine, cytosine arabinoside, cytarabine, azacitidine, 5-azacytosine, gencitabine, 5-azacytosine-arabinoside, vincristine, vinblastine, vinorelbine, leurosine, leurosidine and vindesine, paclitaxel, taxotere and docetaxel.

### Kits

The invention also provides kits. The kits include the compounds and compositions as described herein, contained in a container, and written material that can include instructions for use, discussion of clinical studies, listing of indications, and the like. In some embodiments, the agents in the combinations described herein are provided as separate compositions in separate containers within the kit. In some embodiments, the agents in the combinations described herein are provided as a single composition within a container in the kit. Suitable packaging and additional articles for use (e.g., measuring cup for liquid preparations, foil wrapping to minimize exposure to air, and the like) are known in the art and may be included in the kit.

### Methods

In one aspect the invention is directed to methods of treating subject in need thereof by administering to the subject an effective amount of any of the compounds described herein or their pharmaceutically acceptable salts to the subject. In one embodiment the disorder is a viral disease.

In some embodiments the invention is directed to methods of treating a viral disease comprising administering to a subject in need thereof an effective amount of a compound of formula (A-I) or a pharmaceutically acceptable salt thereof:

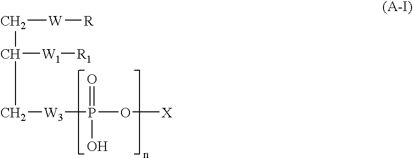

wherein

### W is —S, —SO, —O, —S—C═C, —SC(O), —O—C═C, —OC(O);

### W₁ is —S, —SO, —O, —S—C═C, —SC(O), —O—C═C, —OC(O);

W₃ is —O— or a covalent bond;
n is 1, 2 or 3;
R is C1-C7, C8-C12, C13-C18, or C19-C24 cyclic, straight-chained or branched, unsaturated or saturated alkyl which can be optionally substituted;
R₁ is C1, C2, C3-C10, C11-C15, C16-C17, or C18-C24 cyclic, straight-chained or branched, unsaturated or saturated alkyl which can be optionally substituted; and
X is a nucleoside or nucleoside analog radical.

In some embodiments the invention is directed to methods of treating a viral disease comprising administering to a subject in need thereof an effective amount of a compound of formula (A-VI), (A-VII), (A-VIII), (A-IX) or (A-X), or pharmaceutically acceptable salts thereof:

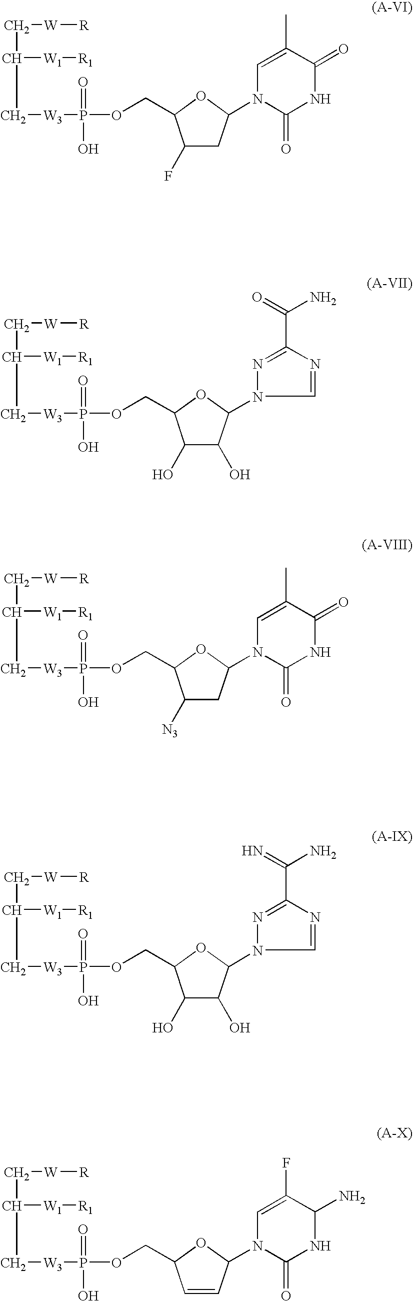

wherein W, W₁, W₃, R, and R₁ are defined and described in formula (A-I) above.

In some embodiments, compounds or pharmaceutically acceptable salts thereof of formula (A-II), (A-VII), (A-VIII), (A-IX) or (A-X) are administered to a subject suffering from a viral disorder.

In some embodiments the invention is directed to methods of treating a viral disease comprising administering to a subject in need thereof an effective amount of a compound of formula (A-II) or a pharmaceutically acceptable salt thereof

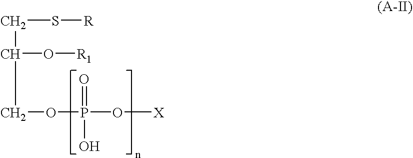

wherein n, R, R₁ and X are defined and described in formula (A-I) above.

In some embodiments, compounds or pharmaceutically acceptable salts thereof of formula (A-II) are administered to a subject suffering from a viral disorder.

In some embodiments the invention is directed to methods of treating a viral disease comprising administering to a subject in need thereof an effective amount of a compound of formula (A-III) or a pharmaceutically acceptable salt thereof:

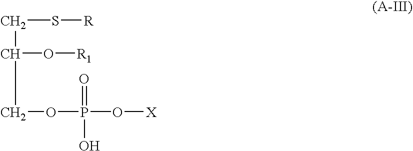

wherein R, R₁ and X are defined and described in formula (A-I) above.

In some embodiments, compounds or pharmaceutically acceptable salts thereof of formula (A-III) are administered to a subject suffering from a viral disorder.

In some embodiments the invention is directed to methods of treating a viral disease comprising administering to a subject in need thereof an effective amount of a compound of formula (A-IV) or a pharmaceutically acceptable salt thereof:

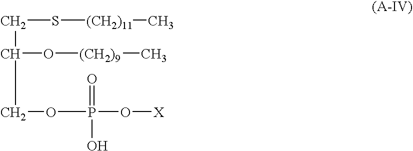

wherein X is defined and described in formula (A-I) above.

In some embodiments, compounds or pharmaceutically acceptable salts thereof of formula (A-IV) are administered to a subject suffering from a viral disorder.

In some embodiments the invention is directed to methods of treating a viral disease comprising administering to a subject in need thereof an effective amount of a compound of formula (A-V) or a pharmaceutically acceptable salt thereof:

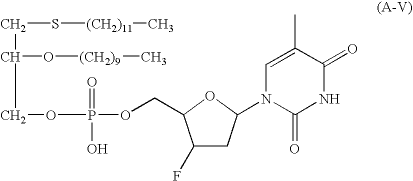

In some embodiments, compounds or pharmaceutically acceptable salts thereof of formula (A-V) are administered to a subject suffering from a viral disorder.

In some embodiments the invention is directed to methods of treating a viral disease comprising administering to a subject in need thereof an effective amount of a compound of formula (A-XI) or a pharmaceutically acceptable salt thereof:

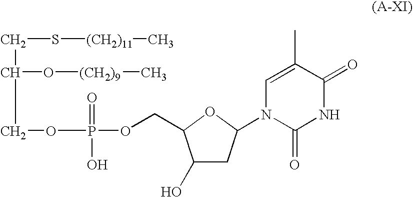

In some embodiments, compounds or pharmaceutically acceptable salts thereof of formula (A-XI) are administered to a subject suffering from a viral disorder.

In some embodiments the invention is directed to methods of treating a viral disease comprising administering to a subject in need thereof an effective amount of a compound of formula (A-XII) or a pharmaceutically acceptable salt thereof:

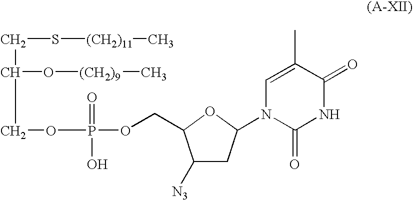

In some embodiments, compounds or pharmaceutically acceptable salts thereof of formula (A-XII) are administered to a subject suffering from a viral disorder.

In some embodiments the invention is directed to methods of treating a viral disease comprising administering to a subject in need thereof an effective amount of a compound of formula (A-XIII) or a pharmaceutically acceptable salt thereof:

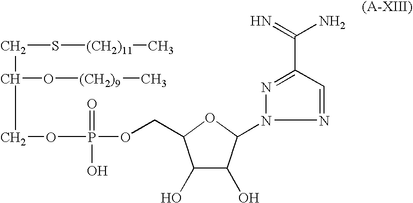

In some embodiments, compounds or pharmaceutically acceptable salts thereof of formula (A-XIII) are administered to a subject suffering from a viral disorder.

In some embodiments the invention is directed to methods of treating a viral disease comprising administering to a subject in need thereof an effective amount of a compound of formula (A-XIV) or a pharmaceutically acceptable salt thereof:

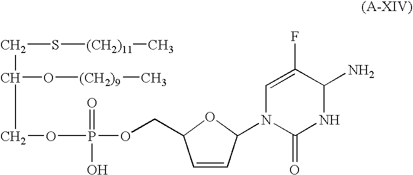

In some embodiments, compounds or pharmaceutically acceptable salts thereof of formula (A-XIV) are administered to a subject suffering from a viral disorder.

In another aspect the invention is directed to methods of treating a viral disease comprising administering to a subject in need thereof an effective amount of general formula (A-I) and another agent such as a nucleoside or a nucleoside analog. Example of agents include but are not limited to nucleosides, nucleosides analogs, interferons such as α, β or γ-interferon, renal excretion inhibitors such as probenecid, nucleoside transport inhibitors such as dipyridamole, immunomodulators such as interleukin II (IL2) and granulocyte macrophage colony stimulating factor (GM-C SF), erythropoetin, empligen, thymomudulin, thymopentin, foscarnet, ribavirin and inhibitors of HIV binding to CD4 receptors e.g. soluble CD4, CD4 fragments, CD4 hybrid molecules, glycosylation inhibitors such as 2-deoxy-D-glucose, castanospermine and 1-deoxynojirimycin.

In some embodiments, the other agent is a nucleoside analog. In some embodiments, the nucleoside or nucleoside analog is cytidine, a cytidine analog, uridine, a uridine analog, adenosine, an adenosine analog, guanosine, a guanosine analog, thymidine, a thymidine analog, inosine or an inosine analog. Without being limited to any theory, the use of combinations of compounds may give rise to an equivalent antiviral effect with reduced toxicity, or an increase in drug efficacy if synergy between compounds occurs.

In some embodiments, the invention is directed to methods of treating a viral disease comprising administering to a subject in need thereof an effective amount of a compound of formula (A-I) and an effective amount of cytidine or a cytidine analog. Examples of cytidine analogs include, but are not limited to, deoxycytidine; 2′,3′-dideoxycytidine; 2′,3′-didehydrocytidine carbocyclic; 2′,3′-didehydro-2′,3′-dideoxycytidine (D4C); 2′,3′-didehydro-2′,3′-dideoxy-5-methylcytidine (D4MeC); fluoro-2′,3′-dideoxycytidine (5-F-ddC); 3-(4-hydroxy-1′,2′-butadienyl)cytosine; 3′-azido-2′,3′-dideoxy-5-methylcytosine (AzddMeC); 3′-azido-2′,3′-dideoxy-5-methylcytosine (AzddMeC); 3′-azido-2′,3′-dideoxy-5-methylcytosine-N4-OH (AzddMeC N4-OH); 3′-azido-2′,3′-dideoxy-5-methylcytosine-N4Me, (AzddMeC N4Me); 3′-azido-2′,3′-dideoxycytosine (AzddC); 3′-azido-2′,3′-dideoxy-5-fluorocytosine (AzddFC); 2′,3′-dideoxy-2′,3′-didehydrocytidine; and beta-L-5-fluoro-2′,3′-dideoxy-2′,3′-didehydrocytidine.

In some embodiments, the cytidine analog is lamivudine, racivir, elvucitabine, apricitabine or emtricitabine. In some embodiments, the cytidine analog is lamivudine. In some embodiments, the cytidine analog is racivir or emtricitabine. In some embodiments, the cytidine analog is elvucitabine. In some embodiments, the cytidine analog is apricitabine.

In some embodiments, the invention is directed to methods of treating a viral disease comprising administering to a subject in need thereof an effective amount of a compound of formula (A-I) or a pharmaceutically acceptable salt thereof and an effective amount of uridine or a uridine analog. Examples of uridine analogs include, but are not limited to, deoxyuridine; 5-Methyluridine; 3′-azido-2′,3′-dideoxy-5-chlorouridine (AzddClU); 3′-azido-2′,3′-dideoxy-5-ethyluridine (AzddEtU); 3′-azido-2′,3′-dideoxyuridine (AzddU); 3′-fluoro-2′,3′-dideoxy-5-bromouridine (3′FddBrU); 3′-fluoro-2′,3′-dideoxy-5-ethyluridine (3′FddEtU); 3′-azido-2′,3′-dideoxy-5-bromouridine (AzddBrU); 3′-azido-2′,3′-dideoxyuridine (AzddIU); 3′-fluoro-2′,3′-dideoxy-5-chlorouridine (FddClU); 3′-fluoro-2′,3′-dideoxyuridine (3′FddU); 2′,3′-dideoxy-3′-azidouridine; and 2′,3′-dideoxy-3′-3′-fluoro-5-chlorouridine.

In some embodiments, the invention is directed to methods of treating a viral disease comprising administering to a subject in need thereof an effective amount of a compound of formula (A-I) and an effective amount of adenosine or an adenosine analog. Example of adenosine analogs include, but are not limited to, deoxyadenosine; 2′,3′-dideoxyadenosine; 2′,3′-dideoxy-2′-fluoro-ara-adenosine; 2-chlorodeoxyadenosine; 9-(4-hydroxy-1′,2′-butadienyl)adenine; 9-(2-phosphonomethoxyethyl)adenine; 2′,3′-didehydro-2′,3′-dideoxyadenosine (D4A); dideoxyadenosine (ddA); 5-methyl-2′,3′-dideoxyadenosine (ddMeA); 3′-fluoro-2′,3′-dideoxy-arabinofuranosyl-adenine (3-Fddara-A); 3′-fluoro-2′,3′-dideoxyadenosine (3-FddA); 2′,3′-dideoxy-2′,3′-didehydro-N6-(O-methylbenzyl)adenosine; 2′,3′-dideoxy-2′,3′-didehydro-N6-(2-methylpropyl)adenosine; and 2′,3′-dideoxy-3′-fluoroadenosine.

In some embodiments, the invention is directed to methods of treating a viral disease comprising administering to a subject in need thereof an effective amount of a compound of formula (A-I) and an effective amount of guanosine or a guanosine analog. Examples of guanosine analogs include, but are not limited to, of deoxyguanosine; 2′,3′-dideoxyguanosine; 2′,3′-didehydroguanosine; 3′-azido-3′-deoxyguanosine; 3′-fluoro-2′,3′-dideoxyguanosine; dideoxyguanosine (ddG); 3′-azideo-2′,3′-dideoxyguanosine (3-N.sub.3 ddG); 3′-fluoro-2′,3′-dideoxyguanosine (3-FddG); and 2′,3′-dideoxy-3′-azidoguanosine. In another embodiment, X is a guanosine radical.

In yet another embodiment, the invention is directed to methods of treating a viral disease comprising administering to a subject in need thereof an effective amount of a compound of formula (A-I) and an effective amount of thymidine or a thymidine analog. Examples of thymidine analogs include, but are not limited to, deoxythymidine; 3′-deoxythymidine; 2′,3′-dideoxythymidine; 2′,3′-didehydrothymidine; 3′-azido-3′-deoxythymidine; 3′-fluoro-3′-deoxythymidine; 3′-fluoro-2′,3′-dideoxythymidine (3′FddT); 3′-deoxy-2′,3′-didehydrothymidine; and 2′,3′-didehydro-2′,3′-dideoxythymidine (D4T). In another embodiment, X is a thymidine radical.

In some embodiments, the invention is directed to methods of treating a viral disease comprising administering to a subject in need thereof an effective amount of a compound of formula (A-I) and an effective amount of inosine or an inosine analog. Examples of inosine analogs include, but are not limited to, deoxyinosine; dideoxyinosine (ddI); and 2′,3′-dideoxyinosine.

In some embodiments the invention is directed to methods of treating a viral disease comprising administering to a subject in need thereof an effective amount of formula (A-I) and an effective amount of a nucleoside analog. In some embodiments, the nucleoside analog is 2,6-diaminopurine-2′,3′-dideoxyriboside; 2,6-diaminopurine-3′-azido-2′,3′-dideoxyriboside; 2,6-diaminopurine-3′-fluoro-2′,3′-dideoxyriboside; 3-phosphonomethoxyethyl-2,6-diaminopurine; 2,6-diaminopurine-2′,3′-dideoxyriboside (ddDAPR); 3′-azido-2′,3′-dideoxy-diaminopurine (N3 ddDAPR); 3′-fluoro-2′,3′-dideoxy-diaminopurine (3-FddDAPR); or 2′,3′-dideoxy-3′-fluoro-2,6-diaminopurineriboside.

In some embodiments, the invention is directed to methods of treating a viral disease comprising administering to a subject in need thereof an effective amount of formula (A-I) and an effective amount of a nucleoside analog. In some embodiments, the nucleoside analog is Abacavir, Aciclovir, Adefovir, Alovudine, Amantadine, amprenavir, Cidofovir, Cytarabine, Desciclovir, Didanosine, Docosanol, Edoxudine, Elvucitabine, Emtricitabine, Famciclovir, Fomivirsen, Foscarnet, Ganciclovir, Idoxuridine, Lamivudine, Oseltamivir, Penciclovir, Peramivir, Rimantadine, Ribavirin, Stavudine, Tenofovir, Tenofovir, Fiacitabine, Fialuridine, doxuridine, Foscamet, Lobucavir, Sorivudine, Trifluridine, Tromantadine, ribavirine, stavudine, Valaciclovir, Valganciclovir, Vidarabine, Viramidine, Zalcitabine, Zanamivir, and Zidovudine.

The invention herein includes pharmaceutically acceptable salts thereof of the nucleoside and nucleoside analogs described above.

In another aspect the invention is directed to methods of treating a viral disease comprising administering to a subject in need thereof an effective amount of a compound of general formula (B-I) or (B-I-A).

In another aspect the invention is directed to methods of treating a viral disease comprising administering to a subject in need thereof an effective amount of a specific enantiomer of a compound of general formula (B-I) or (B-I-A), wherein said specific enantiomer has a desirable effect, e.g, less toxicity when compared to other enantiomer or racemic mixture.

In one embodiment the invention is directed to methods of treating a viral disease comprising administering to a subject in need thereof an effective amount of a compound or a pharmaceutically acceptable salt thereof of formula (B-I) or (B-I-A) to the subject:

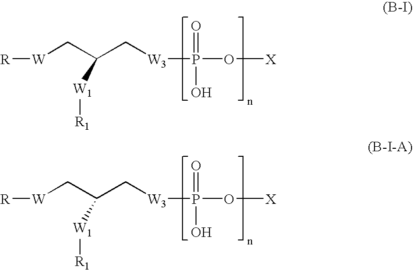

wherein W, W₁, W₃, n, R, R₁ and X are defined and described above. In some embodiments, the compounds or pharmaceutically acceptable salts thereof of formula (B-I) are administered to subjects suffering from a viral disorder.

In some of these embodiments, a pharmaceutically acceptable excipient is also included.

In some embodiments, the invention is directed to methods of treating a viral disease comprising administering to a subject in need thereof an effective amount of a compound or a pharmaceutically acceptable salt thereof of formula (B-II), (B-III), (B-IV), (B-V), (B-VI), (B-VII), (B-VIII), (B-IX), (B-X), (B-XI), (B-XII), (B-XIII) or (B-XIV).

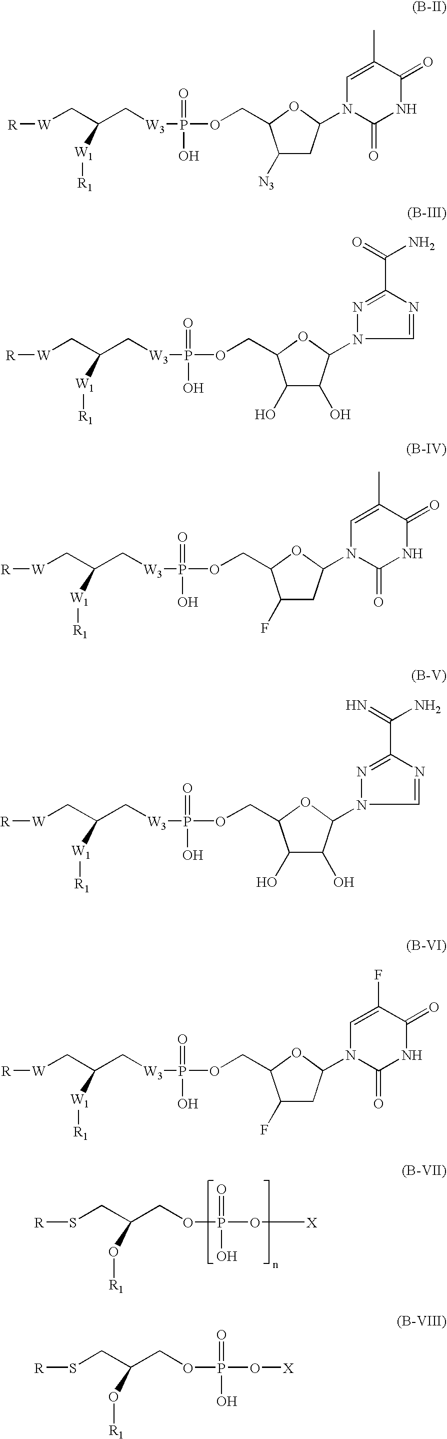

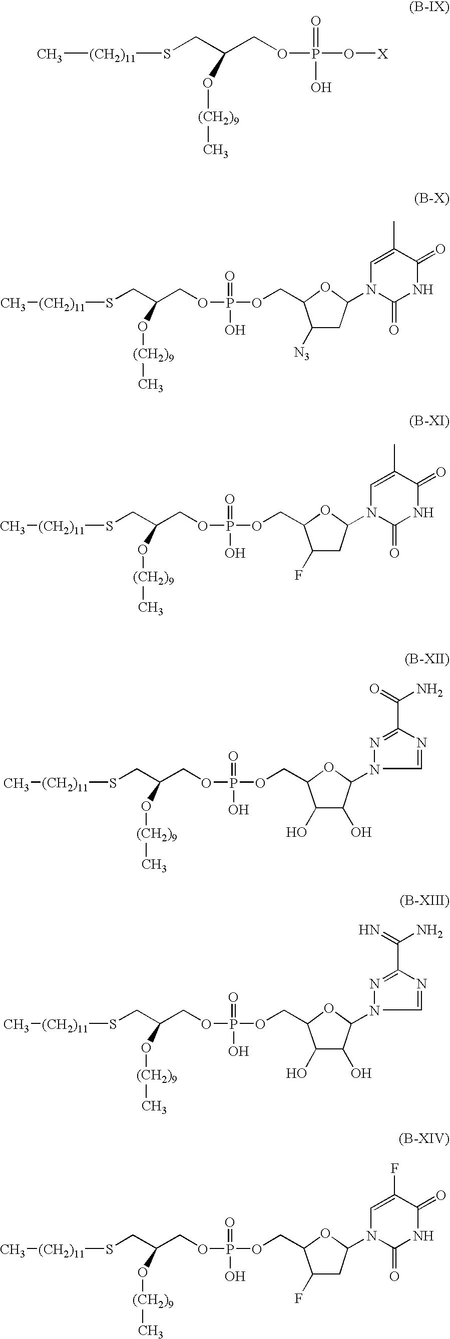

wherein W, W₁, W₃, R, R₁ and X are defined and described in formula (B-I) above.

In some embodiments the compounds are administered to a subject suffering from a viral disorder.

In another aspect the invention is directed to methods of treating a viral disease comprising administering to a subject in need thereof an effective amount of a compound of general formula (B-I) or (B-I-A) and another agent such as a nucleoside or a nucleoside analog. Example of agents include but are not limited to nucleosides, nucleosides analogs, interferons such as α, β or γ-interferon, renal excretion inhibitors such as probenecid, nucleoside transport inhibitors such as dipyridamole, immunomodulators such as interleukin II (IL2) and granulocyte macrophage colony stimulating factor (GM-CSF), erythropoetin, empligen, thymomudulin, thymopentin, foscarnet, ribavirin and inhibitors of HIV binding to CD4 receptors e.g. soluble CD4, CD4 fragments, CD4 hybrid molecules, glycosylation inhibitors such as 2-deoxy-D-glucose, castanospermine and 1-deoxynojirimycin.

In some embodiments, the other agent is a nucleoside or nucleoside analog. In some embodiments, the nucleoside or nucleoside analog is cytidine, a cytidine analog, uridine, a uridine analog, adenosine, an adenosine analog, guanosine, a guanosine analog, thymidine, a thymidine analog, inosine or an inosine analog. Without being limited to any theory, the use of combinations of compounds may give rise to an equivalent antiviral effect with reduced toxicity, or an increase in drug efficacy if synergy between compounds occurs.

In some embodiments, the invention is directed to methods of treating a viral disease comprising administering to a subject in need thereof an effective amount of a compound of formula (B-I) or (B-I-A) and an effective amount of cytidine or a cytidine analog. Examples of cytidine analogs include, but are not limited to, deoxycytidine; 2′,3′-dideoxycytidine; 2′,3′-didehydrocytidine carbocyclic; 2′,3′-didehydro-2′,3′-dideoxycytidine (D4C); 2′,3′-didehydro-2′,3′-dideoxy-5-methylcytidine (D4MeC); fluoro-2′,3′-dideoxycytidine (5-F-ddC); 3-(4-hydroxy-1′,2′-butadienyl)cytosine; 3′-azido-2′,3′-dideoxy-5-methylcytosine (AzddMeC); 3′-azido-2′,3′-dideoxy-5-methylcytosine (AzddMeC); 3′-azido-2′,3′-dideoxy-5-methylcytosine-N4-OH (AzddMeC N4-OH); 3′-azido-2′,3′-dideoxy-5-methylcytosine-N4Me, (AzddMeC N4Me); 3′-azido-2′,3′-dideoxycytosine (AzddC); 3′-azido-2′,3′-dideoxy-5-fluorocytosine (AzddFC); 2′,3′-dideoxy-2′,3′-didehydrocytidine; and beta-L-5-fluoro-2′,3′-dideoxy-2′,3′-didehydrocytidine.

In some embodiments, the cytidine analog is lamivudine, racivir, elvucitabine, apricitabine or emtricitabine. In some embodiments, the cytidine analog is lamivudine. In some embodiments, the cytidine analog is racivir or emtricitabine. In some embodiments, the cytidine analog is elvucitabine. In some embodiments, the cytidine analog is apricitabine.

In some embodiments, the invention is directed to methods of treating a viral disease comprising administering to a subject in need thereof an effective amount of a compound of formula (B-I) or (B-I-A) and an effective amount of uridine or a uridine analog. Examples of uridine analogs include, but are not limited to, deoxyuridine; 5-Methyluridine; 3′-azido-2′,3′-dideoxy-5-chlorouridine (AzddClU); 3′-azido-2′,3′-dideoxy-5-ethyluridine (AzddEtU); 3′-azido-2′,3′-dideoxyuridine (AzddU); 3′-fluoro-2′,3′-dideoxy-5-bromouridine (3′FddBrU); 3′-fluoro-2′,3′-dideoxy-5-ethyluridine (3′FddEtU); 3′-azido-2′,3′-dideoxy-5-bromouridine (AzddBrU); 3′-azido-2′,3′-dideoxyuridine (AzddIU); 3′-fluoro-2′,3′-dideoxy-5-chlorouridine (FddClU); 3′-fluoro-2′,3′-dideoxyuridine (3′FddU); 2′,3′-dideoxy-3′-azidouridine; and 2′,3′-dideoxy-3′-3′-fluoro-5-chlorouridine.

In some embodiments, the invention is directed to methods of treating a viral disease comprising administering to a subject in need thereof an effective amount of a compound of formula (B-I) or (B-I-A) and an effective amount of adenosine or an adenosine analog. Example of adenosine analogs include, but are not limited to, deoxyadenosine; 2′,3′-dideoxyadenosine; 2′,3′-dideoxy-2′-fluoro-ara-adenosine; 2-chlorodeoxyadenosine; 9-(4-hydroxy-1′,2′-butadienyl)adenine; 9-(2-phosphonomethoxyethyl)adenine; 2′,3′-didehydro-2′,3′-dideoxyadenosine (D4A); dideoxyadenosine (ddA); 5-methyl-2′,3′-dideoxyadenosine (ddMeA); 3′-fluoro-2′,3′-dideoxy-arabinofuranosyl-adenine (3-Fddara-A); 3′-fluoro-2′,3′-dideoxyadenosine (3-FddA); 2′,3′-dideoxy-2′,3′-didehydro-N6-(O-methylbenzyl)adenosine; 2′,3′-dideoxy-2′,3′-didehydro-N6-(2-methylpropyl)adenosine; and 2′,3′-dideoxy-3′-fluoroadenosine.

In some embodiments, the invention is directed to methods of treating a viral disease comprising administering to a subject in need thereof an effective amount of a compound of formula (B-I) or (B-I-A) and an effective amount of guanosine or a guanosine analog. Examples of guanosine analogs include, but are not limited to, of deoxyguanosine; 2′,3′-dideoxyguanosine; 2′,3′-didehydroguanosine; 3′-azido-3′-deoxyguanosine; 3′-fluoro-2′,3′-dideoxyguanosine; dideoxyguanosine (ddG); 3′-azideo-2′,3′-dideoxyguanosine (3-N.sub.3 ddG); 3′-fluoro-2′,3′-dideoxyguanosine (3-FddG); and 2′,3′-dideoxy-3′-azidoguanosine.

In some embodiments, the invention is directed to methods of treating a viral disease comprising administering to a subject in need thereof an effective amount of a compound of formula (B-I) or (B-I-A) and an effective amount of thymidine or a thymidine analog. Examples of thymidine analogs include, but are not limited to, deoxythymidine; 3′-deoxythymidine; 2′,3′-dideoxythymidine; 2′,3′-didehydrothymidine; 3′-azido-3′-deoxythymidine; 3′-fluoro-3′-deoxythymidine; 3′-fluoro-2′,3′-dideoxythymidine (3′FddT); 3′-deoxy-2′,3′-didehydrothymidine; and 2′,3′-didehydro-2′,3′-dideoxythymidine (D4T).

In some embodiments, the invention is directed to methods of treating a viral disease comprising administering to a subject in need thereof an effective amount of a compound of formula (B-I) or (B-I-A) and an effective amount of inosine or an inosine analog. Examples of inosine analogs include, but are not limited to, deoxyinosine; dideoxyinosine (ddI); and 2′,3′-dideoxyinosine.

In some embodiments, the invention is directed to methods of treating a viral disease comprising administering to a subject in need thereof an effective amount of a compound of formula (B-I) or (B-I-A) and an effective amount of a nucleoside analog. In some embodiments, the nucleoside analog is 2,6-diaminopurine-2′,3′-dideoxyriboside; 2,6-diaminopurine-3′-azido-2′,3′-dideoxyriboside; 2,6-diaminopurine-3′-fluoro-2′,3′-dideoxyriboside; 3-phosphonomethoxyethyl-2,6-diaminopurine; 2,6-diaminopurine-2′,3′-dideoxyriboside (ddDAPR); 3′-azido-2′,3′-dideoxy-diaminopurine (N3 ddDAPR); 3′-fluoro-2′,3′-dideoxy-diaminopurine (3-FddDAPR); or 2′,3′-dideoxy-3′-fluoro-2,6-diaminopurineriboside.

In some embodiments, the invention is directed to methods of treating a viral disease comprising administering to a subject in need thereof an effective amount of a compound of formula (B-I) or (B-I-A) and an effective amount of a nucleoside analog. In some embodiments, the nucleoside analog is Abacavir, Aciclovir, Adefovir, Alovudine, Amantadine, amprenavir, Cidofovir, Cytarabine, Desciclovir, Didanosine, Docosanol, Edoxudine, Elvucitabine, Emtricitabine, Famciclovir, Fomivirsen, Foscarnet, Ganciclovir, Idoxuridine, Lamivudine, Oseltamivir, Penciclovir, Peramivir, Rimantadine, Ribavirin, Stavudine, Tenofovir, Tenofovir, Fiacitabine, Fialuridine, doxuridine, Foscamet, Lobucavir, Sorivudine, Trifluridine, Tromantadine, ribavirine, stavudine, Valaciclovir, Valganciclovir, Vidarabine, Viramidine, Zalcitabine, Zanamivir, anord Zidovudine.

The invention herein includes pharmaceutically acceptable salts thereof of the nucleoside and nucleoside analogs described above.

The methods, compounds and compositions of the present invention are useful for treating a viral infection caused by chronic hepatitis B, DNA viruses, such as e.g. herpes simplex virus, the cytomegalovirus, Papovavirus, the varicella zoster virus or Epstein-Barr virus, RNA viruses, such as togaviruses or retroviruses, oncoviruses, HTLV-I., HTLV-II, lentiviruses, the clinical manifestations of retroviral HIV infection in humans, such as persistent generalized lymphadenopathy (PGL), the advanced state of AIDS-related complex (ARC) and the clinically complete picture of AIDS, HIV-1, HIV-2.

In one aspect, the invention provides methods of inhibiting viral replication in a cell comprising administering to a subject a composition described herein in an amount sufficient to inhibit viral replication in a cell of the subject.

In one aspect the invention is directed to methods of treating a subject in need of thereof comprising administering an effective amount of the compounds of formula (C-I), including, pharmaceutically acceptable salts thereof in combination with an effective amount of a nucleoside or nucleoside analog or pharmaceutically acceptable salt thereof. In some embodiments, the nucleoside or nucleoside analog is cytidine, a cytidine analog, uridine, a uridine analog, adenosine, an adenosine analog, guanosine, a guanosine analog, thymidine, a thymidine analog, inosine or an inosine analog.

In one embodiment, the invention is directed to methods of treating a subject in need of thereof comprising administering an effective amount of an AZT phospholipid derivative and an effective amount of cytidine or a cytidine analog. Examples of cytidine analogs include, but are not limited to, deoxycytidine; 2′,3′-dideoxycytidine; 2′,3′-didehydrocytidine carbocyclic; 2′,3′-didehydro-2′,3′-dideoxycytidine (D4C); 2′,3′-didehydro-2′,3′-dideoxy-5-methylcytidine (D4MeC); fluoro-2′,3′-dideoxycytidine (5-F-ddC); 3-(4-hydroxy-1′,2′-butadienyl)cytosine; 3′-azido-2′,3′-dideoxy-5-methylcytosine (AzddMeC); 3′-azido-2′,3′-dideoxy-5-methylcytosine (AzddMeC); 3′-azido-2′,3′-dideoxy-5-methylcytosine-N4-OH (AzddMeC N4-OH); 3′-azido-2′,3′-dideoxy-5-methylcytosine-N4Me, (AzddMeC N4Me); 3′-azido-2′,3′-dideoxycytosine (AzddC); 3′-azido-2′,3′-dideoxy-5-fluorocytosine (AzddFC); 2′,3′-dideoxy-2′,3′-didehydrocytidine; and beta-L-5-fluoro-2′,3′-dideoxy-2′,3′-didehydrocytidine.

In some embodiments, the cytidine analog is lamivudine, racivir, elvucitabine, apricitabine or emtricitabine. In some embodiments, the cytidine analog is lamivudine. In some embodiments, the cytidine analog is racivir or emtricitabine. In some embodiments, the cytidine analog is elvucitabine. In some embodiments, the cytidine analog is apricitabine.

In another embodiment, the invention is directed to methods of treating a subject in need of thereof comprising administering an effective amount of an AZT phospholipid derivative in combination with an effective amount of uridine or a uridine analog. Examples of uridine analogs include, but are not limited to, deoxyuridine; 5-Methyluridine; 3′-azido-2′,3′-dideoxy-5-chlorouridine (AzddClU); 3′-azido-2′,3′-dideoxy-5-ethyluridine (AzddEtU); 3′-azido-2′,3′-dideoxyuridine (AzddU); 3′-fluoro-2′,3′-dideoxy-5-bromouridine (3′FddBrU); 3′-fluoro-2′,3′-dideoxy-5-ethyluridine (3′FddEtU); 3′-azido-2′,3′-dideoxy-5-bromouridine (AzddBrU); 3′-azido-2′,3′-dideoxyuridine (AzddIU); 3′-fluoro-2′,3′-dideoxy-5-chlorouridine (FddClU); 3′-fluoro-2′,3′-dideoxyuridine (3′FddU); 2′,3′-dideoxy-3′-azidouridine; and 2′,3′-dideoxy-3′-3′-fluoro-5-chlorouridine.

In another embodiment, the invention is directed to methods of treating a subject in need of thereof comprising administering an effective amount of an AZT phospholipid derivative in combination with an effective amount of adenosine or an adenosine analog. Example of adenosine analogs include, but are not limited to, deoxyadenosine; 2′,3′-dideoxyadenosine; 2′,3′-dideoxy-2′-fluoro-ara-adenosine; 2-chlorodeoxyadenosine; 9-(4-hydroxy-1′,2′-butadienyl)adenine; 9-(2-phosphonomethoxyethyl)adenine; 2′,3′-didehydro-2′,3′-dideoxyadenosine (D4A); dideoxyadenosine (ddA); 5-methyl-2′,3′-dideoxyadenosine (ddMeA); 3′-fluoro-2′,3′-dideoxy-arabinofuranosyl-adenine (3-Fddara-A); 3′-fluoro-2′,3′-dideoxyadenosine (3-FddA); 2′,3′-dideoxy-2′,3′-didehydro-N6-(O-methylbenzyl)adenosine; 2′,3′-dideoxy-2′,3′-didehydro-N6-(2-methylpropyl)adenosine; and 2′,3′-dideoxy-3′-fluoroadenosine.

In another embodiment, the invention is directed to methods of treating a subject in need of thereof comprising administering an effective amount of an AZT phospholipid derivative in combination with an effective amount of guanosine or a guanosine analog. Examples of guanosine analogs include, but are not limited to, of deoxyguanosine; 2′,3′-dideoxyguanosine; 2′,3′-didehydroguanosine; 3′-azido-3′-deoxyguanosine; 3′-fluoro-2′,3′-dideoxyguanosine; dideoxyguanosine (ddG); 3′-azideo-2′,3′-dideoxyguanosine (3-N.sub.3 ddG); 3′-fluoro-2′,3′-dideoxyguanosine (3-FddG); and 2′,3′-dideoxy-3′-azidoguanosine.

In yet another embodiment, the invention is directed to methods of treating a subject in need of thereof comprising administering an effective amount of an AZT phospholipid derivative in combination with an effective amount of thymidine or a thymidine analog. Examples of thymidine analogs include, but are not limited to, deoxythymidine; 3′-deoxythymidine; 2′,3′-dideoxythymidine; 2′,3′-didehydrothymidine; 3′-azido-3′-deoxythymidine; 3′-fluoro-3′-deoxythymidine; 3′-fluoro-2′,3′-dideoxythymidine (3′FddT); 3′-deoxy-2′,3′-didehydrothymidine; and 2′,3′-didehydro-2′,3′-dideoxythymidine (D4T).

In yet another embodiment, the invention is directed to methods of treating a subject in need of thereof comprising administering an effective amount of an AZT phospholipid derivative in combination with an effective amount of inosine or an inosine analog. Examples of inosine analogs include, but are not limited to, deoxyinosine; dideoxyinosine (ddI); and 2′,3′-dideoxyinosine.

In some embodiments the invention is directed to methods of treating a subject in need of thereof comprising administering an effective amount of an AZT phospholipid derivative and an effective amount of a nucleoside analog. In some embodiments, the nucleoside analog is 2,6-diaminopurine-2′,3′-dideoxyriboside; 2,6-diaminopurine-3′-azido-2′,3′-dideoxyriboside; 2,6-diaminopurine-3′-fluoro-2′,3′-dideoxyriboside; 3-phosphonomethoxyethyl-2,6-diaminopurine; 2,6-diaminopurine-2′,3′-dideoxyriboside (ddDAPR); 3′-azido-2′,3′-dideoxy-diaminopurine (N3 ddDAPR); 3′-fluoro-2′,3′-dideoxy-diaminopurine (3-FddDAPR); or 2′,3′-dideoxy-3′-fluoro-2,6-diaminopurineriboside.

In other embodiments, the invention is directed to methods of treating a subject in need of thereof comprising administering an effective amount of an AZT phospholipid derivative and an effective amount of a nucleoside analog. In some embodiments, the nucleoside analog is Abacavir, Aciclovir, Adefovir, Alovudine, Amantadine, amprenavir, Cidofovir, Cytarabine, Desciclovir, Didanosine, Docosanol, Edoxudine, Elvucitabine, Emtricitabine, Famciclovir, Fomivirsen, Foscarnet, Ganciclovir, Idoxuridine, Lamivudine, Oseltamivir, Penciclovir, Peramivir, Rimantadine, Ribavirin, Stavudine, Tenofovir, Tenofovir, Fiacitabine, Fialuridine, doxuridine, Foscamet, Lobucavir, Sorivudine, Trifluridine, Tromantadine, ribavirine, stavudine, Valaciclovir, Valganciclovir, Vidarabine, Viramidine, Zalcitabine, Zanamivir, or Zidovudine.

In another aspect the invention is directed to methods of treating subject in need of thereof comprising administering an effective amount of a compound of structure (C-I) or a pharmaceutically acceptable salt thereof and an effective amount of a compound of structure (C-II) or a pharmaceutically acceptable salt thereof. In some embodiments, the compound of structure (C-II) is a mixture of cis-isomers. In some embodiments, the compound of structure (C-II) is a mixture of trans-isomers. In some embodiments, the compound of structure (C-II) is the (cis)-isomer in the form of a single enantiomer. In some embodiments, the compound of structure (C-II) is the (trans)-isomer in the form of a single enantiomer. In some embodiments, the compound of structure (C-II) is in the form of the negative enantiomer. In some embodiments, the compound of structure (C-II) is in the form of the positive enantiomer.

In another aspect the invention is directed to methods of treating subject in need of thereof comprising administering an effective amount of a compound of structure (C-I) or a pharmaceutically acceptable salt thereof and an effective amount of a compound of structure (C-III) or a pharmaceutically acceptable salt thereof.

In another aspect the invention is directed to methods of treating subject in need of thereof by administering an effective amount of a compound of structure (C-I) or a pharmaceutically acceptable salt thereof and an effective amount of a compound of structure (C-IV) or a pharmaceutically acceptable salt thereof. In some embodiments the compound of structure (C-IV) is a mixture of the cis-isomers. In some embodiments, the compound of structure (C-IV) is (cis)-isomer in the form of a single enantiomer. In some embodiments, the compound of structure (C-IV) is the (1S,4R)-isomer.

In another aspect the invention is directed to methods of treating subject in need of thereof comprising administering an effective amount of a compound of structure (C-I) or a pharmaceutically acceptable salt thereof and an effective amount of a compound of structure (C-V) or a pharmaceutically acceptable salt thereof. In some embodiments, the compound of structure (C-V) is a mixture of cis-isomers. In some embodiments, the compound of structure (C-V) is a mixture of trans-isomers. In some embodiments, the compound of structure (C-V) is the (cis)-isomer in the form of a single enantiomer. In some embodiments, the compound of structure (C-V) is the (trans)-isomer in the form of a single enantiomer. In some embodiments, the compound of structure (C-V) is in the form of the negative enantiomer. In some embodiments, the compound of structure (C-V) is in the form of the positive enantiomer.

In another aspect the invention is directed to methods of treating subject in need of thereof comprising administering an effective amount of a compound of structure (C-I) or a pharmaceutically acceptable salt thereof and an effective amount of a compound of structure (C-VI) or a pharmaceutically acceptable salt thereof. In some embodiments, the compound of structure (C-IV) is a mixture of cis-isomers. In some embodiments, the compound of structure (C-VI) is a mixture of trans-isomers. In some embodiments, the compound of structure (C-VI) is the (cis)-isomer in the form of a single enantiomer. In some embodiments, the compound of structure (C-VI) is the (trans)-isomer in the form of a single enantiomer. In some embodiments, the compound of structure (C-VI) is in the form of the negative enantiomer. In some embodiments, the compound of structure (C-VI) is in the form of the positive enantiomer.

The invention herein includes pharmaceutically acceptable salts of the nucleoside and nucleoside analogs described above.

### Administration

The methods described herein comprise the administration of the compounds and compositions described herein. In some embodiments, other agents are also administered. In some embodiments, the other agents are therapeutic agents. When two or more agents are co-administered, they may be co-administered in any manner, such as, though not limited to separate compositions, in the same composition, by the same or by different routes of administration.

In some aspects, the methods herein involve the administration of a compound of general formula (A-I). In some aspects, the methods herein involve the administration of a compound of general formula (A-I), to treat viral infections. In some aspects, the methods herein involve the administration of a compound of general formula (A-I), to treat HIV infections. In some embodiments, a nucleoside or nucleoside analog is administered in combination with a compound of general formula (A-I). In some embodiments, other agents are also administered, e.g., other therapeutic agents. When two or more agents are co-administered, they may be co-administered in any manner, such as, but not limited to separate compositions, in the same composition, by the same or by different routes of administration.

In some embodiments, a compound of general formula (A-I) is administered in a single dose. In some embodiments, such administration is oral, e.g., in a tablet. However, other routes may be used as appropriate. A single dose of a nucleoside analog may also be used when it is administered with a compound of general formula (A-I) for treatment of a condition.

In some embodiments, a compound of general formula (A-I) is administered in multiple doses. Dosing may be about once, twice, three times, four times, five times, six times, or more than six times per day. Dosing may be about once a month, once every two weeks, once a week, or once every other day. In some embodiments, a compound of general formula (A-I) is administered about once per day to about 6 times per day. In some embodiments, the administration of a compound of general formula (A-I) continues for less than about 7 days. In some embodiments, the administration continues for more than about 6, 10, 14, 28 days, two months, six months, or one year. In some cases, continuous dosing is achieved and maintained as long as necessary, e.g., through the life of the subject. In some embodiments, a compound of general formula (A-I) is administered continually, e.g. with a minipump, patch or stent.

Administration of a compound of general formula (A-I) may continue as long as necessary, e.g., through the life of the subject. In some embodiments, an agent of the invention is administered for more than 1, 2, 3, 4, 5, 6, 7, 14, 28 days or 1 year. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic effects.

An effective amount of a compound of general formula (A-I) may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent.

A compound of general formula (A-I) may be administered in dosages as described herein (see, e.g., Compositions). Dosing ranges for compounds of general formula (A-I) are known in the art. It is also known in the art that due to intersubject variability in compounds of general formula (A-I), pharmacokinetics, individualization of dosing regimen is necessary for optimal therapy. Dosing for a compound of general formula (A-I) may be found by routine experimentation.

In some aspects, the methods herein involve the administration of a compound of general formula (B-I) or (B-I-A), e.g., to treat HIV infections. In some embodiments, a nucleoside or nucleoside analog is administered in combination with a compound of general formula (B-I) or (B-I-A). In some embodiments, other agents are also administered, e.g., other therapeutic agent. When two or more agents are co-administered, they may be co-administered in any manner, such as but not limited to separate compositions, in the same composition, by the same or by different routes of administration.

In some embodiments, a compound of general formula (B-I) or (B-I-A) is administered in a single dose. In some embodiments, such administration is oral, e.g., in a tablet. However, other routes may be used as appropriate. A single dose of a nucleoside analog may also be used when it is administered with a compound of general formula (B-I) or (B-I-A) for treatment of a condition.

In some embodiments, a compound of general formula (B-I) or (B-I-A) is administered in multiple doses. Dosing may be about once, twice, three times, four times, five times, six times, or more than six times per day. Dosing may be about once a month, once every two weeks, once a week, or once every other day. In some embodiments, a compound of general formula (B-I) or (B-I-A) is administered about once per day to about 6 times per day. In some embodiments, the administration of a compound of general formula (B-I) or (B-I-A) continues for less than about 7 days. In some embodiments, the administration continues for more than about 6, 10, 14, 28 days, two months, six months, or one year. In some cases, continuous dosing is achieved and maintained as long as necessary, e.g., through the life of the subject. In some embodiments, a compound of general formula (B-I) or (B-I-A) is administered continually, e.g. with a minipump, patch or stent.

Administration of a compound of general formula (B-I) or (B-I-A) may continue as long as necessary, e.g., through the life of the subject. In some embodiments, an agent of the invention is administered for more than 1, 2, 3, 4, 5, 6, 7, 14, 28 days or 1 year. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic effects.

An effective amount of a compound of general formula (B-I) or (B-I-A) may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent.

A compound of general formula (B-I) or (B-I-A) may be administered in dosages as described herein (see, e.g., Compositions). Dosing ranges for compounds of general formula (B-I) or (B-I-A) are known in the art. It is also known in the art that due to intersubject variability in compounds of general formula (B-I) or (B-I-A), pharmacokinetics, individualization of dosing regimen is necessary for optimal therapy. Dosing for a compound of general formula (B-I) or (B-I-A) may be found by routine experimentation.

In some embodiments, the compound of structure (C-I) is administered in a single dose. The administration may be oral, e.g., in a tablet. The administration may be via other routes, as appropriate. A single dose of a compound of structure (C-II), (C-III), (C-IV) (C-V) or (C-VI) may also be used when it is administered with the compound of structure (C-I) for treatment of a condition.

In some embodiments, the compound of structure (C-I) is administered in multiple doses. Dosing may be about once, twice, three times, four times, five times, six times, or more than six times per day. Dosing may be about once a month, once every two weeks, once a week, or once every other day. In some embodiments the compound of structure (C-II), (C-III), (C-IV) (C-V) or (C-VI) and the compound of structure (C-I) are administered together about once per day to about 6 times per day. In another embodiment the administration of the composition continues for less than about 7 days. In yet another embodiment the administration continues for more than about 6, 10, 14, 28 days, two months, six months, or one year. In some cases, continuous dosing is achieved and maintained as long as necessary, e.g., through the life of the subject. In some embodiments, the nucleoside analog and/or AZT phospholipid derivative is administered continually, e.g. with a minipump, patch or stent.

Administration of the compositions may continue as long as necessary, e.g., through the life of the subject. In some embodiments, the composition is administered for more than 1, 2, 3, 4, 5, 6, 7, 14, 28 days or 1 year. In some embodiments, the composition is administered for less than 28, 14, 7, 6, 5, 4, 3, 2, or 1 day. In some embodiments, the composition is administered chronically on an ongoing basis, e.g., for the treatment of chronic effects.

The composition may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent.

The compositions may be administered in dosages as described herein. Dosing ranges for compounds of formula (C-I) are known in the art. It is also known in the art that due to intersubject variability, individualization of dosing regimen may be necessary for optimal therapy.

In some embodiments the compounds and compositions described herein are useful for the treatment of cancer.

### Synthetic Procedures

In another aspect, the invention relates to methods for making the compounds described herein. In some embodiments, the compounds described herein can be prepared by the methods described below. The compounds of formula (C-VII) can be prepared according to the following Scheme in which W, W₁, R and R₁ have the meanings described herein. The starting alcohols are synthesized as previously described. See. M. Marx et al., J. Med. Chem. 31, 858 (1988); S. Morris-Natschke et al., J. Med. Chem. 29, 2114 (1986).

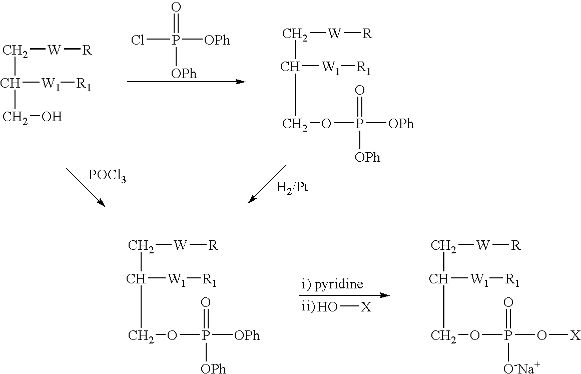

### Preparation of Phospholipid Nucleoside Conjugates

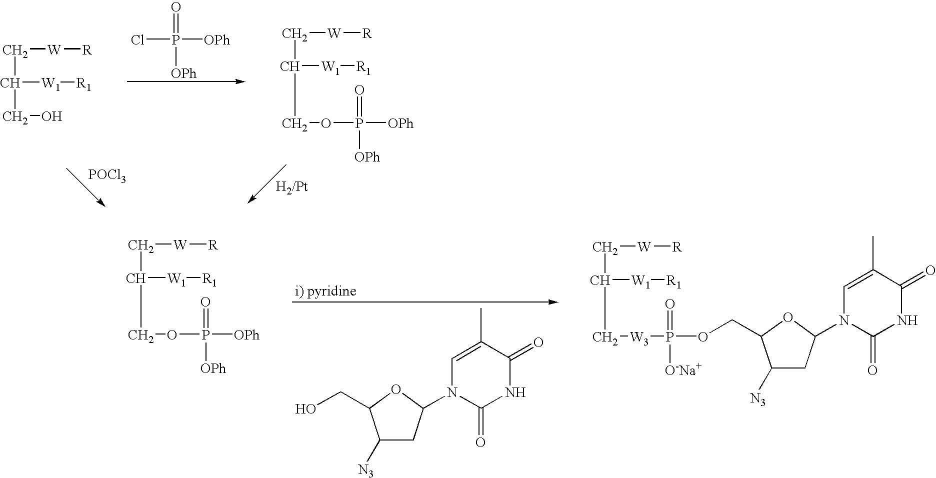

### Preparation of the Compounds of Formula (C-VII)

The amidoalkyl glycerol derivative is phosphorylated with diphenylchlorophosphate in pyridine to give the corresponding phosphate ester. See C. Piantadosi, J. Pharm. Sci. 62, 320 (1973). The phenyl groups are then removed via hydrogenolysis with PtO₂ to give the intermediate. The thio and oxygen ether derivatives are phosphorylated by an alternative procedure using phosphorus oxychloride and triethylamine or pyridine. See Ether Lipids: Biochemical and Biomedical Aspects, 403 (H. Mayold and F. Paltauf eds. 1983); C. Hong et al., J. Med. Chem. 29, 2038 (1986). The phosphatidic acid derivatives are then conjugated to the 5′ hydroxyl of the appropriate nucleoside (NUC) via. dicyclohexylcarbodiimide (DCC) condensation, and subsequent conversion to the sodium salt gave the desired products. See E. Ryu et al., J. Med. Chem. 25, 1322 (1982).

The compounds described herein can also be made by reacting the compound below

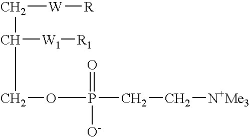

in which W, W₁, R and R₁ have the meanings described herein, with a nucleoside or nucleoside analog, as described above, in the presence of phospholipase D in a inert solvent, such as e.g. chloroform, in the presence of a buffer and, after reaction has taken place, possibly removes any oxygen protective group corresponding to processes usual in nucleoside chemistry. For additional details see Lipids, 1997, 22, 947, DE-A-3039629, and EP-A-0350287.

The compounds of the present invention may have asymmetric carbon atoms. Diastereomeric mixtures can be separated into their individual diastereomers on the basis of their physical chemical differences by methods known to those skilled in the art, for example, by chromatography or fractional crystallization.

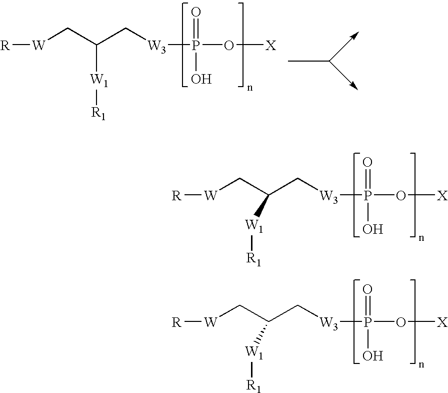

Enantiomers can be separated by converting the enantiomer mixture into a diastereomeric mixture by reaction with an appropriate optically active compound (e.g. alcohol), separating the diastereomers and converting (e.g., hydrolyzing) the individual diastereomers to the corresponding pure enantiomers. Other methods such as chiral chromatography for preparing optically active isomers are also contemplated. All such isomers, including diastereomeric mixtures and pure enantiomers are considered as part of the invention.

### EXAMPLES

### Example 1

### (3′-Deoxy-3′-Azidothymidine)-5′-Phosphoric Acid (3-Dodecylthio-2-ethyloxy)-Propyl Ester

POCl₃ (0.42 ml, 4.5 mmol) is added dropwise to a solution of 3-dodecylthio-2-ethyloxy-1-propanol (1.25 g, 3 mmol) and triethylamine (1.2 ml, 8.6 mmol) in absolute ether (40 ml) under nitrogen, at 0° C. and stirring continued for 45 min. The solution is allowed to warm to room temperature and a solution 3′-deoxy-3′-azidothymidine (AZT, 800 mg, 3 mmol) in absolute ether (15 ml) and absolute toluene (20 ml) is added dropwise and stirred for 6 hours under reflux (TLC control). After cooling, water (50 ml) is added and the mixture stirred vigorously for 2 hours The organic phase thereafter is isolated, dried over Na₂ SO₄ and evaporated in a rotary evaporator. The residue is purified by preparative column chromatography on silica gel 60 with dichloromethane/methanol 9:1 as eluent to provide (3′-Deoxy-3′-Azidothymidine)-5′-Phosphoric Acid (3-Dodecylthio-2-ethyloxy)-Propyl Ester.

### Example 2

### (3′-Deoxy-3′-Azidothymidine)-5′-Phosphoric acid (3-Dodecylthio-2-ethyloxy)-Propyl Ester

AZT (2 mmol) and phospholipase D (5000 U) are suspended in sodium acetate buffer/CaCl₂ (4 ml) and mixed with a solution of 3-dodecylthio-2-ethyloxypropyl-1-phosphoric acid monocholine ester (6 mmol) in chloroform (160 ml). The mixture is heated for 8 hours at 45° C., dried over Na₂SO₄ and the solvent removed under vacuum. The residue is purified by column chromatography as in Example 1 to provide (3′-Deoxy-3′-Azidothymidine)-5′-Phosphoric acid (3-Dodecylthio-2-ethyloxy)-Propyl Ester. (See Chem. Pharm. Bull. 1988, 36, 5020.)

### Example 3

### (2′,3′-Dideoxy-2′,3′-didehydro-N₆—(O-methylbenzyl)-adenosine)-5′-phosphoric acid (3-dodecylthio-2-ethyloxy)-propyl ester

Phosphoric acid (3-dodecylthio-2-ethyloxy)-propyl ester (680 mg, 1.37 mmol) in absolute pyridine (20 ml) are mixed with 2′,3′-dideoxy-2′,3′-didehydro-N₆—(O-methylbenzyl)-adenosine (337 mg, 1 mmol) and DCC (1.37 g, 6.7 mmol). The mixture is stirred for 24 hours at room temperature (TLC control). The pyridine is removed under vacuum and the residue suspended in ether. Undissolved urea is removed by fitration and the filtrate purified, after the evaporation of the solvent, by column chromatography on silica gel 60 with dichloromethane/methanol 95/5 as eluent to provide (2′,3′-Dideoxy-2′,3′-didehydro-N₆—(O-methylbenzyl)-adenosine)-5′-phosphoric acid (3-dodecylthio-2-ethyloxy)-propyl ester.

### Example 4

### (3′-Deoxy-3′-fluorothymidine)-5′-phosphoric acid (3-dodecylthio-2-ethyloxy)-propyl ester

The title compound is prepared following a similar method as described in example 3, using phosphoric acid (3-dodecylthio-2-ethyloxy)-propyl ester (13.5 g), 3′-deoxy-3′-fluorothymidine (5.4 g) and DCC (27 g) in absolute pyridine (350 ml) and the product purified as described above to provide (3′-Deoxy-3′-fluorothymidine)-5′-phosphoric acid (3-dodecylthio-2-ethyloxy)-propyl ester.

### Example 5

### (3′-Deoxythymidine)-5′-phosphoric acid 3-dodecylthio-2-ethyloxy)-propyl ester

The title compound is prepared following a similar method as described in example 3, using phosphoric acid (3-dodecylthio-2-ethyloxy)-propyl ester (1.3 g), 3′-deoxythymidine (500 mg) and DCC (2.6 g) in absolute pyridine (40 ml). The mixture is stirred for 24 hours at room temperature and the product purified by chromatography to provide (3′-Deoxythymidine)-5′-phosphoric acid 3-dodecylthio-2-ethyloxy)-propyl ester.

### Example 6

### (2′,3′-Dideoxyinosine)-5′-phosphoric acid (3-dodecylthio-2-ethyloxy)-propyl ester

The title compound is prepared following a similar method as described in example 3, using phosphoric acid (3-dodecylthio-2-ethyloxy)-propyl ester (1.3 g), 2′,3′-dideoxy-inosine (500 mg) and DCC (2.6 g) in absolute pyridine (40 ml). The mixture is stirred for 40 hours at room temperature and the product purified by chromatography to provide (2′,3′-Dideoxyinosine)-5′-phosphoric acid (3-dodecylthio-2-ethyloxy)-propyl ester.

### Example 7

### (3′-Deoxy-3′-azidothymidine)-5′-phosphoric acid (3-dodecylmercapto-2-decyloxy)-propyl ester

AZT (2 mmol) and phospholipase D (5000 U) are suspended in sodium acetate buffer/CaCl₂ (4 ml) and mixed with a solution of 3-dodecylmercapto-2-decyloxypropyl-1-phosphoric acid monocholine ester (6 mmol) in chloroform (160 ml). The mixture is heated for 8 h. at 45° C., dried over Na₂SO₄ and the solvent removed under vacuum. The residue is purified by column chromatography as in Example 1 to provide (3′-Deoxy-3′-azidothymidine)-5′-phosphoric acid (3-dodecylmercapto-2-decyloxy)-propyl ester. (See Chem. Pharm. Bull. 1988, 36, 5020).

### Example 8

### Synthetic Scheme for Phosphoric Acid (2S,3S,5R)-3-azido-5-(5-methyl-2,4-dioxo-3,4-dihydro-2H-pyrimidin-1-yl)-tetrahydro-furan-2-ylmethyl ester (S)-2-decyloxy-3-dodecylsulfanyl-propyl ester

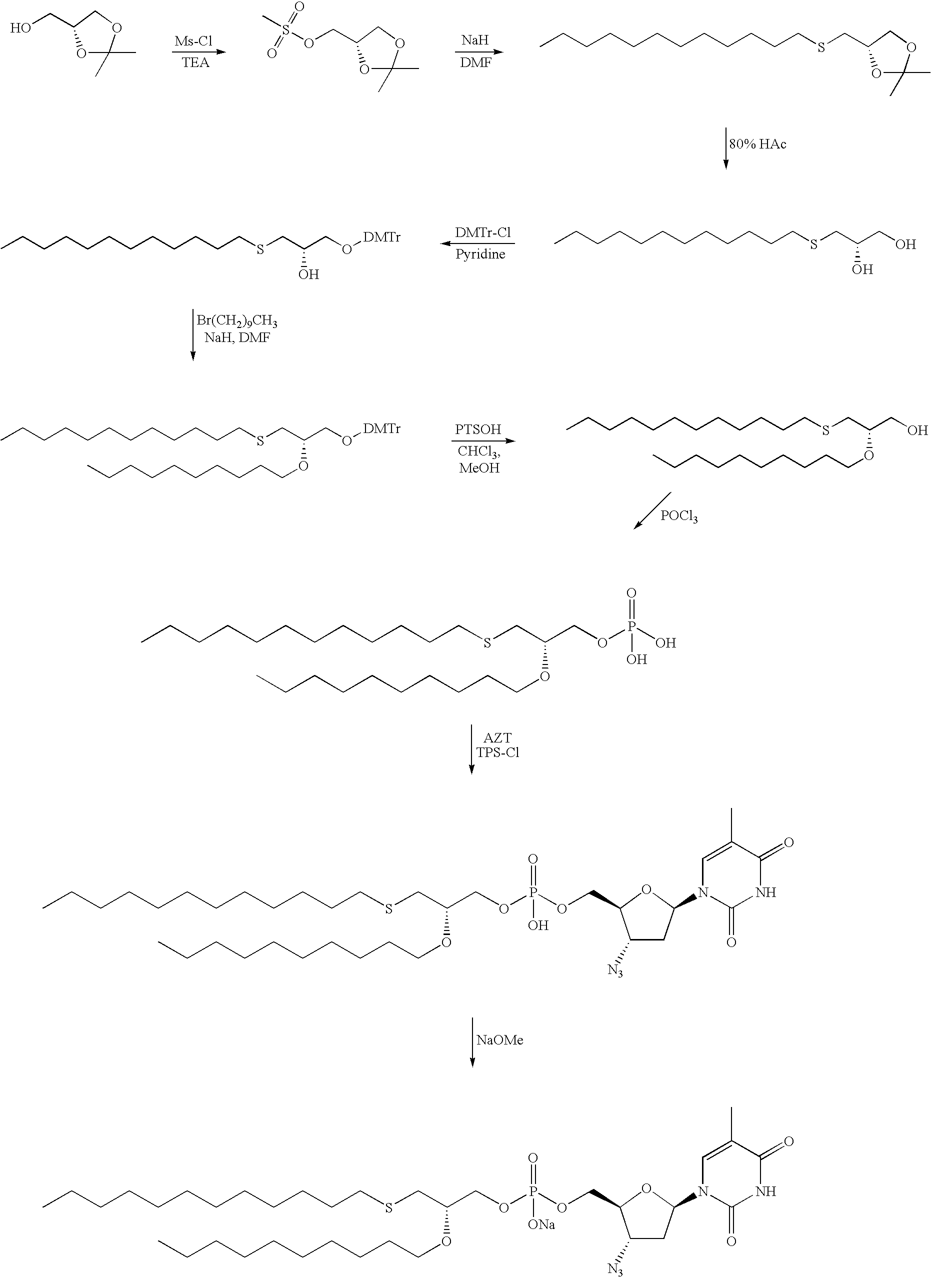

### Example 9

### Synthetic Scheme for Phosphoric Acid (2S,3S,5R)-3-azido-5-(5-methyl-2,4-dioxo-3,4-dihydro-2H-pyrimidin-1-yl)-tetrahydro-furan-2-ylmethyl ester (R)-2-decyloxy-3-dodecylsulfanyl-propyl ester

(FOZ ISO 851 and FOZ ISO 851 Na)

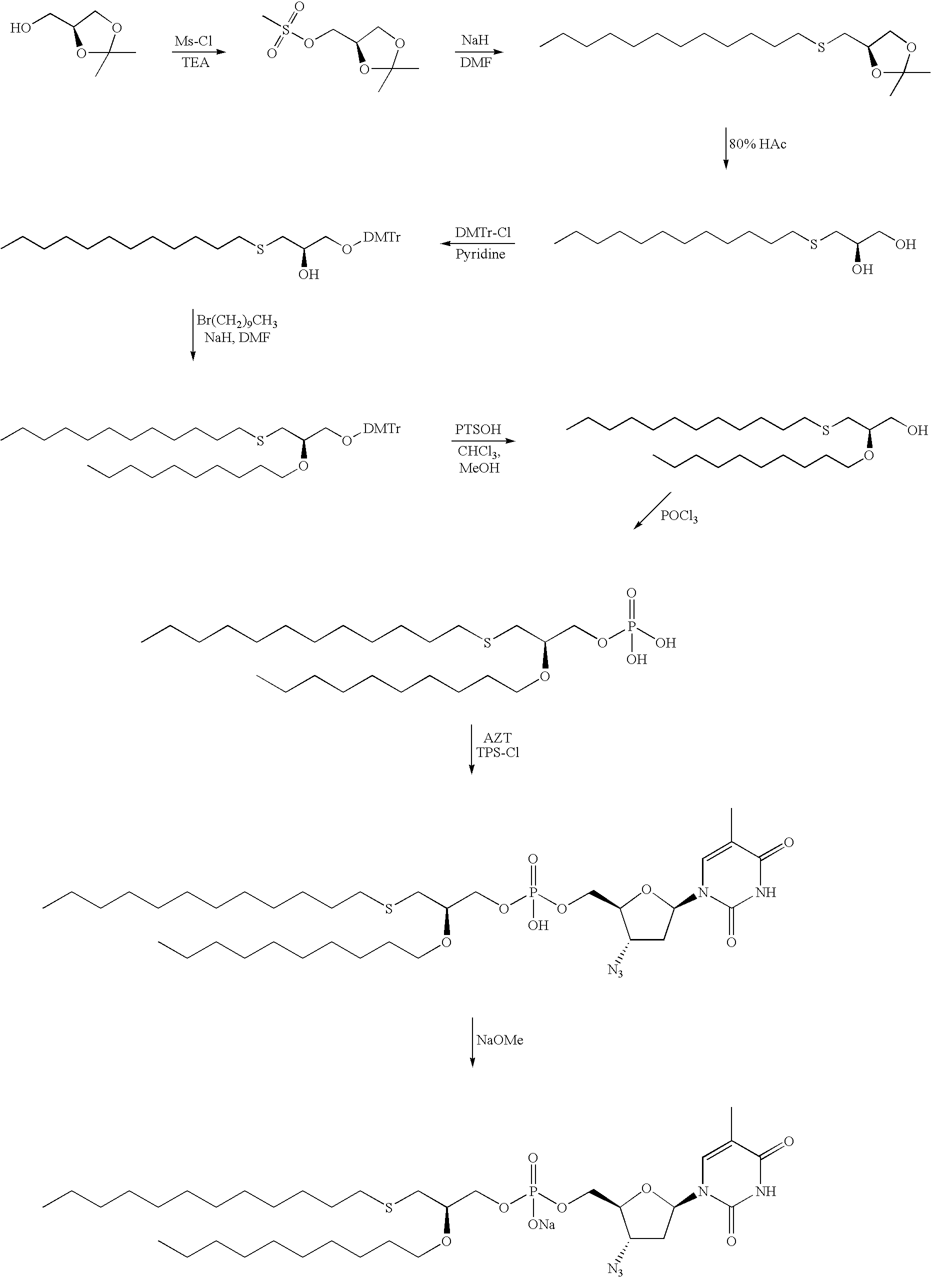

### Example 10

### Synthetic Scheme of Phosphoric Acid (2S,3S,5R)-3-azido-5-(5-methyl-2,4-dioxo-3,4-dihydro-2H-pyrimidin-1-yl)-tetrahydro-furan-2-ylmethyl ester 2-decyloxy-3-dodecylsulfanyl-propyl ester

(FOZ ISO 850 and FOZ ISO 850 Na)

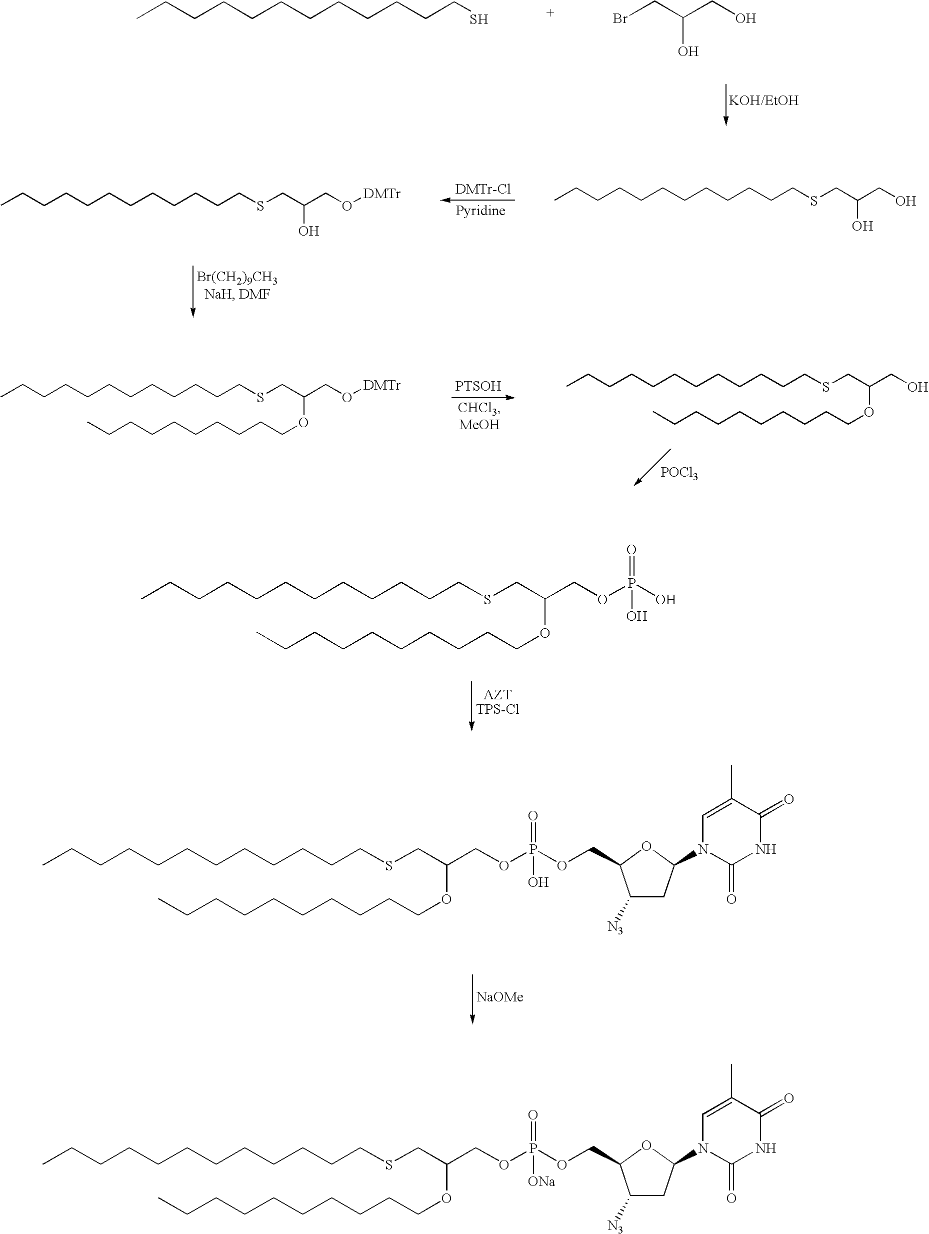

### Example 11

### Preparation of (S)-3-Dodecylthio-propane-1,2-diol

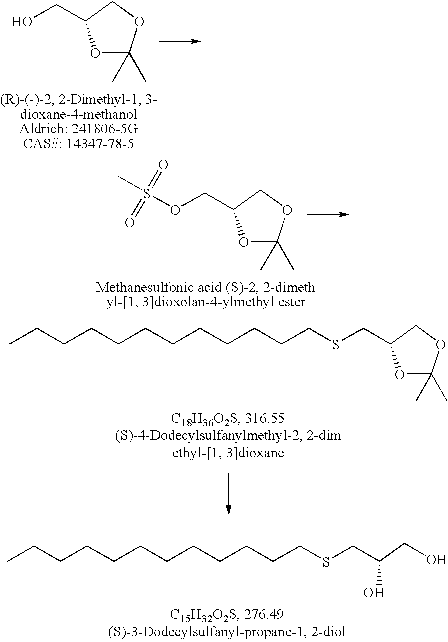

### (1) (S)-(2,2-dimethyl-1,3-dioxolan-4-yl)methyl methanesulfonate

(S)-(2,2-dimethyl-1,3-dioxolan-4-yl)methyl methanesulfonate was prepared as describe in Journal of Medicinal Chemistry, 44(19), 3092-3108; 2001. To an ice-cooled, stirred solution of (R)-(−)-(2,2-dimethyl-1,3-dioxolan-4-yl)methanol (20.3 g, 153.9 mmol) and triethylamine (46.7 g, 461.7 mmol) in methylene chloride (150 mL) was dropwise neat methanesulfonyl chloride (26.4 g, 230.9 mmol). After stirring at 0° C. for 30 min and at room temperature for 3 h, the resulting mixture was diluted with dichloromethane (500 mL) and washed with brine (300 mL), saturated sodium bicarbonate (300 mL) and brine/water (1:1, 150 mL×2), dried over anhydrous sodium sulfate, filtered, and concentrated under reduced pressure to give crude (S)-(2,2-dimethyl-1,3-dioxolan-4-yl)methyl methanesulfonate (32.9 g), which was used in the next step without further purification.

### (2) (S)-4-(Dodecylthiomethyl)-2,2-dimethyl-1,3-dioxolane

(S)-4-(Dodecylthiomethyl)-2,2-dimethyl-1,3-dioxolane was prepared as described in Antiviral Chemistry & Chemotherapy (1998), 9(1), 33-40. 1-Dodecanethiol (31.1 g, 153.9 mmol) was added carefully to a stirred mixture of dry DMF (200 ml) and 60% NaH (12.3 g, 307.8 mmol). A solution of crude (S)-(2,2-dimethyl-1,3-dioxolan-4-yl)methyl methanesulfonate (32.9 g, 153.9 mmol) in 100 ml of dry DMF was then added in one portion. The mixture was stirred at room temperature overnight and at 80° C. for 3 hours. When the reaction was complete, as shown by TLC, the solvent was removed under reduced pressure and the residue was dissolved in dichloromethane then washed with water three times. The organic layer was dried over anhydrous sodium sulfate, filtered, concentrated to dryness to give crude (S)-4-(Dodecylthiomethyl)-2,2-dimethyl-1,3-dioxolane as an oil (51.0 g). A pure sample was obtained by silica gel chromatography (hexane to 9:1 hexane:ethyl acetate). R_{f} 0.85 (2:5 ethyl acetate/hexane).

### (3) (S)-3-Dodecylthio-propane-1,2-diol

The above crude (S)-4-(Dodecylthiomethyl)-2,2-dimethyl-1,3-dioxolane was suspended in 500 ml of 80% acetic acid and stirred for 3 days at room temperature. The solvent was removed under reduced pressure and the residue was co-evaporated with ethanol and re-crystallized from ethanol. The white solid was filtered and washed with cold ethanol twice and dried under reduced pressure at room temperature to give (S)-3-Dodecylthio-propane-1,2-diol (22 g, 52%) as white crystals. The filtrate was evaporated to dryness and re-crystallized from ethanol to give another batch of pure product (15 g).

(R)-3-Dodecylthio-propane-1,2-diol was prepared by the same procedure using (S)-(+)-2,2-dimethyl-1,3-dioxolane-4-methanol as a starting material.

### Example 12

### Preparation of 3-dodecylthio-propane-1,2-diol

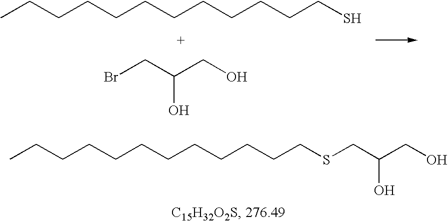

3-dodecylthio-propane-1,2-diol was prepared as described in J. Med. Chem. 2003, 4205. A solution of potassium hydroxide (12.0 g, 213.7 mmol) and 1-dodecanethiol (37.6 g, 185.8 mmol) in 150 ml of ethanol was stirred at room temperature for 30 min. 3-Bromo-1,2-propanediol (29.4 g, 189.5 mmol) was added slowly to the solution and the reaction mixture was stirred for 24 hours. At that time, the precipitate was filtered and re-crystallized from ethyl acetate and a few drops of methanol to yield 3-dodecylthio-propane-1,2-diol (48 g, 93%) as white crystals. R_{f}=0.6 in ethyl acetate/hexane (1:1).

### Example 13

### Preparation of (S)-2-decyloxy-3-dodecylsulfanyl-propan-1-ol

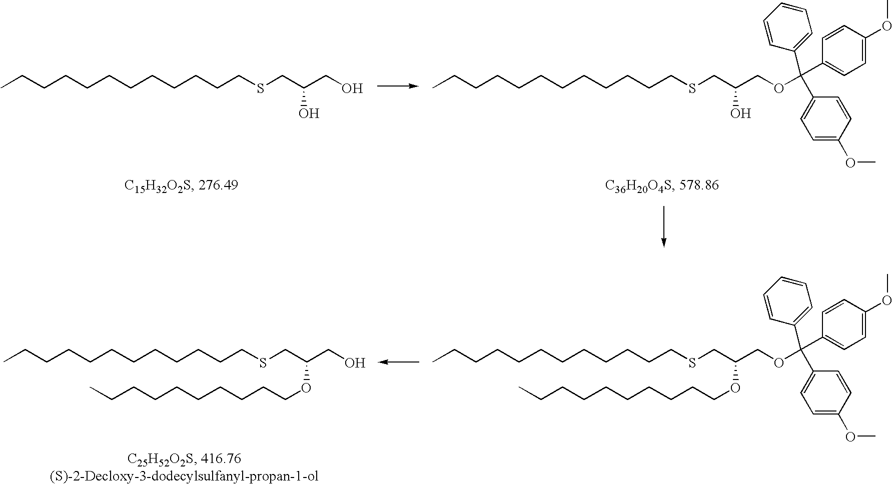

### (1) (S)-1-Dodecylthio-3-(4,4′-dimethoxytrityloxy)-propan-2-ol

A solution of (S)-3-dodecylthio-propane-1,2-diol (21.5 g, 77.8 mmol) in pyridine (150 mL) was added 4,4′-dimethoxytrityl chloride (31.6 g, 93.4 mmol). The mixture was stirred at room temperature for 4 h. Afterwards, the pyridine was removed, and the resulting yellow oil was dissolved in dichloromethane and washed with brine three times. The organic phase was dried over anhydrous sodium sulfate and evaporated to dryness under reduced pressure to give crude products, which was used for next step without further purification.

### (2) (S)-1-dodecylthio-2-decyloxy-3-(4,4′-dimethoxytrityloxy)-propanol

The dried crude product from above was dissolved in 300 ml of dry DMF and added 60% sodium hydride (12.4 g, 311.2 mmol) at room temperature under argon. The mixture was stirred at room temperature for 30 min and then bromodecane (51.6 g, 233.4 mmol) was added. The mixture was stirred for 30 min at room temperature and overnight at 80° C. TLC showed this reaction is complete. The reaction was quenched with water and extracted with diethyl ether three times. The ether solution was washed with water and dried over sodium sulfate and evaporated to dryness under reduced pressure. The residue was purified by silica gel chromatography (ISCO, 330 g of column, hexane/EtOAc, 0-20%, 70 min) to give a yellow oil product.

### (3) (S)-2-decyloxy-3-dodecylsulfanyl-propan-1-ol

(S)-2-decyloxy-3-dodecylsulfanyl-propan-1-ol was prepared as described in Reference: J. Med. Chem. 2003, 4205. (S)-1-dodecylthio-2-decyloxy-3-(4,4′-dimethoxytrityloxy)-propanol (13.5 g, 18.8 mmol) from above was dissolved in chloroform and methanol (180 mL:55 mL). Next, p-toluenesulfonic acid (3.5 g) was added. The reaction mixture was stirred at room temperature for 24 h. Saturated NaHCO₃ solution was added, and the solution stirred for 30 min. After extraction with chloroform, the organic phase was dried over anhydrous Na₂SO₄. Finally, the solvent was removed under reduced pressure, and the resulting oil was purified by silica gel chromatography (ISCO, 120 g of column, 50 min, hexane/EtOAc, 0-20%). The desired product fractions were pooled and dried to yield (S)-2-decyloxy-3-dodecylsulfanyl-propan-1-ol as a colorless oil (R_{f=}0.5 in ethyl acetate/hexane, 1:6).

(R)-2-Decyloxy-3-dodecylsulfanyl-propan-1-ol and 2-Decyloxy-3-dodecylsulfanyl-propan-1-ol were prepared by similar procedures using (R)-3-dodecylthio-propane-1,2-diol and 3-dodecylthio-propane-1,2-diol respectively in place of (S)-3-dodecylthio-propane-1,2-diol.

### Example 14

### Phosphoric Acid (2S,3S,5R)-3-azido-5-(5-methyl-2,4-dioxo-3,4-dihydro-2H-pyrimidin-1-yl)-tetrahydro-furan-2-ylmethyl ester (S)-2-decyloxy-3-dodecylsulfanyl-propyl ester

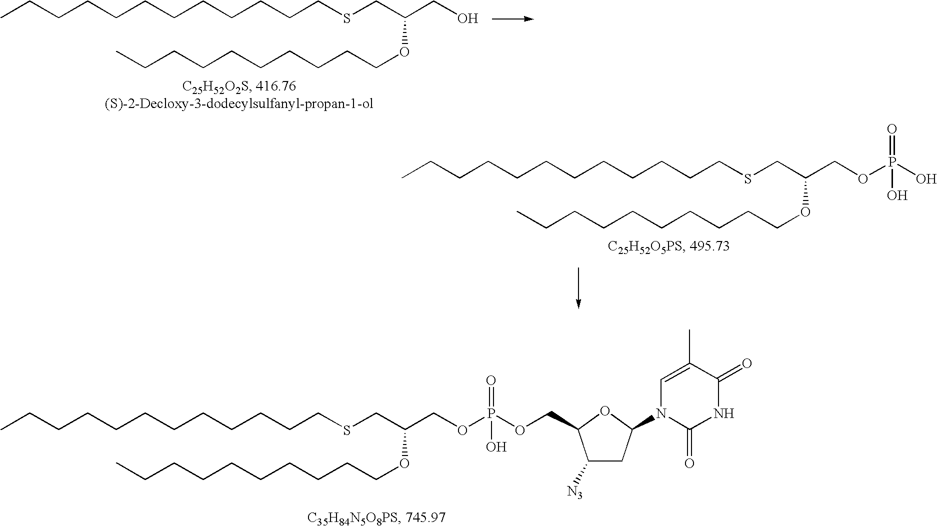

Reference: J. Med. Chem. 2003, 4205

### (1) Phosphoric acid mono-((S)-2-decyloxy-3-dodecylsulfanyl-propyl) ester

Phosphorus oxychloride (0.81 g, 5.3 mmol) was dissolved in 5 mL of THF at 0° C. A solution of (S)-1-dodecylthio-2-decyloxy-propan-3-ol (1.77 g, 4.2 mmol) in 17 mL of THF and 1.1 mL of pyridine was added to the POCl₃ solution at 0° C., and the stirring was continued for 5 h. At this time, 15 mL of a saturated NaHCO₃ solution was added and the mixture stirred for 30 min. Finally, the solution was poured over ice water and acidified with 6N HCl. The solution was extracted with diethyl ether, washed with water, and dried over anhydrous Na₂SO₄. The solvent was removed under reduced pressure, and the crude oily product (2.2 g) was used for next step without further purification. A analytically pure sample was obtained by chromatography on silica gel eluting first with CHCl₃:MeOH:NH₄OH (75:25:5) (500 mL) and finally 100% MeOH. The desired fractions were pooled and solvent evaporated to yield a white paste. R=0.1 in CHCl₃:MeOH:NH₄OH (75:25:5).

### (2) Phosphoric acid (2S,3S,5R)-3-azido-5-(5-methyl-2,4-dioxo-3,4-dihydro-2H-pyrimidin-1-yl)-tetrahydro-furan-2-ylmethyl ester (S)-2-decyloxy-3-dodecylsulfanyl-propyl ester

2,4,6-Triisopropylbenzene-sulfonyl chloride (2.6 g) and 50 mL of pyridine were added to the phosphoric acid (2.2 g) synthesized above, and the pyridine was removed under reduced pressure. Next, pyridine (50 mL) and AZT (1.0 g) were added, and the pyridine was removed under reduced pressure. Finally, fresh pyridine (50 mL) was added and the reaction mixture was heated at 50° C. for overnight. Water was added, and the solvent was removed under vacuum. Diethyl ether was added and the precipitate was filtered and discarded. The solvent was removed and the crude oil was dissolved in methyl tert-butyl ether and washed with 2N HCl and brine and dried over sodium sulfate and evaporated to give a crude oily product, which was purified by silica gel chromatography (ISCO, 80 g of column, chloroform (10 min) and then chloroform/methanol (0-10%, 40 min). The desired product fractions were pooled and evaporated to dryness to yield phosphoric acid (2S,3S,5R)-3-azido-5-(5-methyl-2,4-dioxo-3,4-dihydro-2H-pyrimidin-1-yl)-tetrahydro-furan-2-ylmethyl ester (S)-2-decyloxy-3-dodecylsulfanyl-propyl ester as a white paste (1.78 g, 57%). R_{f}=0.22 in Chloroform/7N NH₃ in Methanol (5:1).

Phosphoric acid (2S,3S,5R)-3-azido-5-(5-methyl-2,4-dioxo-3,4-dihydro-2H-pyrimidin-1-yl)-tetrahydro-furan-2-ylmethyl ester (R)-2-decyloxy-3-dodecylsulfanyl-propyl ester and Phosphoric acid (2S,3S,5R)-3-azido-5-(5-methyl-2,4-dioxo-3,4-dihydro-2H-pyrimidin-1-yl)-tetrahydro-furan-2-ylmethyl ester (R,S)-2-decyloxy-3-dodecylsulfanyl-propyl ester were prepared by similar procedures using (R)-1-dodecylthio-2-decyloxy-propan-3-ol and (R,S)-1-dodecylthio-2-decyloxy-propan-3-ol respectively in place of (R)-1-dodecylthio-2-decyloxy-propan-3-ol.

### Example 15

### Phosphoric acid (2S,3S,5R)-3-azido-5-(5-methyl-2,4-dioxo-3,4-dihydro-2H-pyrimidin-1-yl)-tetrahydro-furan-2-ylmethyl ester (S)-2-decyloxy-3-dodecylsulfanyl-propyl ester sodium salt

### (FOZ MIX Na)

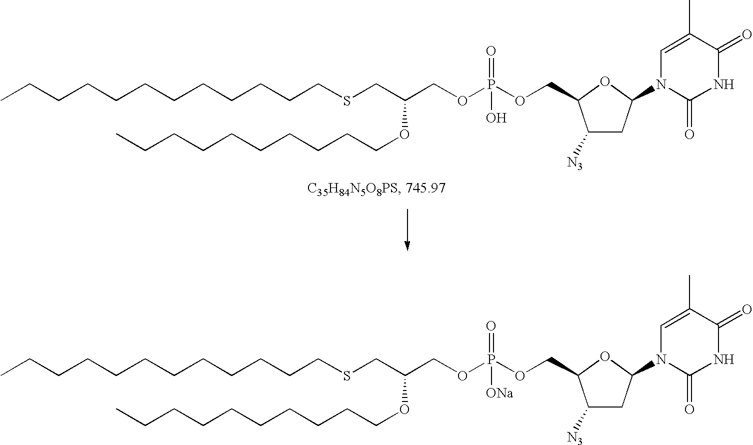

Phosphoric acid (2S,3S,5R)-3-azido-5-(5-methyl-2,4-dioxo-3,4-dihydro-2H-pyrimidin-1-yl)-tetrahydro-furan-2-ylmethyl ester (S)-2-decyloxy-3-dodecylsulfanyl-propyl ester sodium salt was prepared as described in U.S. Pat. No. 5,734,042.

Purified (S)-Fozivudine Tidoxil (408 mg, 0.55 mmol) was dissolved in 3 ml of toluene at room temperature. 0.12 ml of 25% sodium methoxide in methanol was added to the solution. The clear solution was slowly added dropwise to acetone at 0° C. with stirring. It was stirred overnight until the precipitate became powdery. The precipitate was suction filtered and rinsed with a small amount of acetone and the slightly sticky product was dried at 50° C. to give phosphoric acid (2S,3S,5R)-3-azido-5-(5-methyl-2,4-dioxo-3,4-dihydro-2H-pyrimidin-1-yl)-tetrahydro-furan-2-ylmethyl ester (S)-2-decyloxy-3-dodecylsulfanyl-propyl ester sodium salt as a white solid.

Sodium Salts of the (R) and (S,R) isomers were prepared by the same procedures using (R)-Fozivudine Tidoxil and (R,S)-Fozivudine Tidoxil respectively in place of (S)-Fozivudine Tidoxil.

### Example 16

### Inhibition of HIV-1 Reverse Transcriptase

The compounds described herein can be assayed for inhibitory activity against human immunodeficiency virus type 1 (HIV-1) using a high throughput cell-based assay using HIV-1 expressing firefly luciferase as a reporter gene and pseudotyped with vesicular stomatitis virus envelope glycoprotein (VSV-G). Experimental procedures are essentially as described by Connor et al. in Journal of Virology (1996), 70: 5306-5311 (Characterization of the functional properties of env genes from long-term survivors of human immunodeficiency virus type 1 infection), and Popik et al. in Journal of Virology (2002), 76: 4709-4722 (Human immunodeficiency virus type 1 uses lipid raft-co-localized CD4 and chemokine receptors for productive entry into CD4+ T cells). It should be particularly appreciated that the virus contains two introduced mutations in the RT gene (K103N and Y181C, created by PCR mutagenesis) that render the virus highly resistant to current non-nucleoside HIV-1 drugs. Virus stocks are generated by cotransfection of plasmid DNA encoding VSV-G with vector pNL4-3Env(−)Luc(+) into 293T cells. Sixty-four hours after transfection, virus-containing medium is collected by centrifugation and stored frozen at −80° C.

HeLa cells are infected with the VSV-G pseudotyped virus in the presence of screening compounds in a 384-well microtiter plate format. Forty-eight hours after initial infection, lysis buffer and Luciferase Assay Reagent (Promega) are added to the cells and luciferase activity is determined by counting the resultant luminescence using a LJL luminometer. Since the luciferase gene is carried in the virus genome, its expression level directly reflects the virus replication level in the presence of a compound.

To evaluate the activity of the compounds against wild type HIV-1, the HeLa-JC53 cell line that expresses high levels of CD4 and CCR5 (see e.g., Platt et al. in Journal of Virology (1998), 72: 2855-2864:. Effect of CCR5 and CD4 cell surface concentrations on infection by macrophagetropic isolates of human immunodeficiency virus type 1) is modified by isolation of a stable cell line that expresses luciferase under the control of the HIV-1 promoter (long terminal repeat, i.e., LTR). HIV-1 infection of this cell line stimulates the transcription of luciferase from the HIV-1 promoter and the luciferase gene expression level is proportional to the level of virus replication (Harrington et al. in Journal of Virology Methods (2000), 88: 111-115: Direct detection of infection of HIV-1 in blood using a centrifugation-indicator cell assay; and Roos et al. in Virology (2000), 273: 307-315: LuSIV cells: a reporter cell line for the detection and quantitation of a single cycle of HIV and SIV replication). Procedures for virus infection, compound testing and luciferase activity determination are the same as for the VSV-G pseudotyped HIV-1.

Two approaches are used to evaluate the cytotoxicity of the positive compounds discovered in the HIV-1 virus assays. The first approach employs another modified HeLa-JC53 cell line that constitutively expresses high level of luciferase without virus infection. The level of luciferase expression in these cells served as an indicator for cell replication in the presence of the compounds. Procedures for compound testing and luciferase activity determination are the same as for the virus infection tests. The other toxicity assay utilized HeLe-JC53 cells and a commercially available MTS assay kit (Promega) that measures the mitochondria function of the cells.

### Example 17

### Inhibition of Infection of Two Primary HIV-1 Isolates in PBMCs

Monocyte/macrophages represent a major reservoir of HIV-1 in the infected human host. See L. Epstein et al., AIDS Res. 14, 447 (1984). However, these cells tend to be resistant to dideoxynucleoside prodrugs due to low levels of kinases needed to activate the prodrugs. See C. Pemo et al., J. Exp. Med. 168, 1111 (1984). To test the compounds described herein in these cells, PBMC cells infected for 7 days with two different virus strains (20678 and JR-FL) and under PHA stimulation for 4 days were treated with AZT, FOZ ISO 850, FOZ ISO 851, and FOZ MIX Na.

PBMC's were prepared as follows: 50 ml of whole human blood was centrifuged at 1000 g for 15 min, supernatant (plasma) removed, and cells resuspended in 80 ml PBS. 20 ml resuspended blood was then layered onto 25 ml Ficoll-Paque Plus (GE Healthcare, Uppsala Sweden), and centrifuged at 400 g for 30 min. The PBMC layer at the interface was collected, washed two times with 20 ml PBS, and resuspended in 20 ml RPMI/10% FBS.

Day 5-Stimulation of PBMC: PBMCs were grown in 50 ml of stimulation medium (RPMI, 15% FBS, 5 units/ml of IL-2, and 6 ug/ml of PHA) for 5 days.

Day 1-Infection of PBMC: PBMCs were counted with a hemacytometer. Stimulation medium was removed by centrifugation at RT, 1,500 rpm/5′ and cells resuspended in growth medium (RPMI, 15% of FBS, 10 units/ml of IL-2). PBMCs were infected with JR-FL or 20678 as follows: for one 96-well plate, 20 millions cells were infected with 1 ml of each virus (use 10 ul of virus and 200,000 cell per well) in a 4.5 ml-volume in a 50-ml conical tube. Cells were incubated for 3 hrs at 37□ C. with frequent shaking. Aliqouts of 50 ul of a 4× solution of the compounds to be tested were added to the 96-well plates. After 3 hr of incubation, infected cells were washed two times with PBS and one time with culture medium by spinning down the cells at 1500 rpm for 5 minutes at room temperature. After the last wash cells were resuspended in 15 ml of growth medium. 150 ul of cell suspension were delivered into each well. Cells were incubated for 7 days at 37□ C.

Day 5-Medium change: Cells were resuspended by pipetting and 100 ul of medium was removed. 100 ul of fresh medium was added with 1× compound. Cells were incubated for 3 more days at 37° C.

Day 8-p24 assay: At day 8 the p24 production was measured using the HIV-1 p24 Antigen ELISA (Zeptometrix) according to manufacturer instructions.

Tables 1-3 show the results.

TABLE 1

| | |
|---|---|
| PBMC | EC₅₀ (nM) |

| 2 viruses | 20678 | JR-FL |
|---|---|---|
| (AZT) | 76.03 | 89.76 |
| FOZ ISO 850 | 279.3 | 473.8 |
| FOZ ISO 851 | 294.2 | 504 |
| FOZ MIX Na | 1414 | 1137 |

TABLE 2

| | Diff.CD34+ | | |
|---|---|---|---|
| | GI₅₀ (μM) | GI₅₀ (μM | |
| | Aug. 6, 2007 | Aug. 13 2007 | AVG |
| | 7-d | 7-d | GI₅₀ (μM) |
| (AZT) | 0.07 | 0.20 | 0.14 |
| FOZ ISO 850 | 2.2 | 4.0 | 3.09 |
| FOZ ISO 851 | 0.85 | 1.2 | 1.03 |
| FOZ ISO 850 Na | 4.0 | >10 | 4.00 |
| FOZ ISO 851 Na | 2.2 | >10 | 2.20 |
| FOZ MIX Na | 1.5 | 2.2 | 1.83 |

Using the results above a selectivity index can be calculated by dividing GI₅₀ (concentration required for 50% inhibition of cell growth) by EC₅₀. Table 3 shows the selectivity index value.

TABLE 3

| | Selectivity Index |
|---|---|
| (AZT) | 1.8 |
| FOZ ISO 850 | 11.06 |
| FOZ ISO 851 | 3.5 |
| FOZ ISO 850 Na | 14.3 |
| FOZ ISO 851 Na | 4.5 |

### Example 18

### Anti-HBV Activity in HEP-G2 Cells

### Cytotoxicity

Cell cytotoxicity evaluations are performed in 96-well plates seeded with HEP-G2 cell monolayers. The hepatoblastoma cell line HEP-G2 transfected with a plasmid carrying the gene that confers resistance to C418 and four 5′-3′ tandem copies of the Hepatitis B virus genome can be used Acs. Proc. Natl. Acad. Sci. USA, 84, 1005-1009 (1987). Serial concentrations of test compound are added to the RPMI-1640 growth medium and the growth medium is added to the 96-well plates containing the cell monolayers. The cells are incubated for a total of nine days at 37° C. The compound-containing medium is periodically replaced. Each compound is tested at four serial concentrations in triplicate. After nine days of treatment and incubation, 0.01% neutral red dye (Sigma, Inc.) is added to the overlay medium. Cell cytotoxicity is quantitated by measuring the average A₅₁₀. (See also Korba and Germ, Antiviral Res., 19, 55-70 (1992)).

Anti-Hepatitis B Virus Activity in HEP-G2 Cells Persistently Infected with HBV

HEP-G2 cells persistently infected with HBV are treated daily with fresh D-MEM containing 20% FBS and 10 μM compound. After 9 days incubation and treatment, the overlay medium is harvested to assay the quantity of Hepatitis B virus s antigen (HBsAg) and HBeAg by EIA (Abbott Laboratories). The overlay medium is diluted to levels of antigen in the linear range of the assay. Standard curves using dilutions of positive HBsAg and HBeAg controls are included in each assay. Virion DNA is assayed using the method set forth in Korba and Gerin, Antiviral Res., 19, 5, 5-70 (1992). HBV DNA is extracted from the overlay medium containing virus particles and analyzed by a slot-blot hybridization technique using ^{0.32}P-labelled 3.2 Kb Eco R1 HBV DNA fragment by comparisons to HBV standards present on nitrocellulose filters using an AMBIS beta scanner. EC₉₀ values (90% effective concentration, i.e., drug concentration which induces a 90% decrease in the levels of HBV DNA replication intermediates in treated versus control cells) are calculated by linear regression analysis. The relative levels of HBsAg, HBeAg and virion DNA are calculated as a percentage of the average levels obtained from the untreated controls.

### Example 19

### Anti-HBV Activity in HBV Persistently Infected Human Hepatablastoma Cell Line HEP-G2 Cytotoxicity

Cytotoxicity in human HEP-G2 cells persistently infected with HBV is determined using 2′-3′-Dideoxycytidine (ddC) as a control. The toxicity analyses are performed in 96-well flat bottomed tissue culture plates following the same procedure as described herein. Each compound is tested at four concentrations, each in triplicate cultures. Uptake of neutral red dye is used to determine the relative level of toxicity. The absorbance of internalized dye at 510 nM (A_{0.510}) is used for the quantitative analysis.

### Effect on HBV Replication

Cells are treated with a particular compound for nine days at 37° C. Analysis of intracellular HEIV DNA is complete 24 hours following the ninth day of treatment. The levels of integrated HBV DNA in each cellular DNA preparation is used to calculate the levels of episomal 3.2-3.8 Kb HBV genomes (Mono) and HBV DNA replication intermediates (RI). A zero value indicates an undetectable level of HBV virion DNA. The sensitivity cut-off is usually 0.1 pg DNA/ml of culture medium.

### Effect on Antigen Markers

Cells are treated with compound for nine days at 37° C. The overlay medium is harvested to measure levels of HBsAg, HBeAg and virion DNA. HBsAg and HBeAg are assayed by EIA (Abbott Laboratories). Virion DNA levels are measured as described herein.

### Example 20

### In Vivo Evaluation of Cytotoxicity and Bioavailability

### Biologic Activity

Sprague Dawley rats are fasted for 18 hours and receive a single oral dose of 15 mg/kg of the compound. An in-dwelling carotid catheter is used to harvest whole blood at various times post-dose. The plasma containing the compound is diluted 1:2 to 1:128 in phosphate buffered saline and assayed for biologic activity using the HIV-1 syncytial plaque test. CEM-SS cell monolayers are infected with HIV-1 and overlaid with RPMI-1640 medium containing serial dilutions of plasma. After incubation for five days at 37° C., the number of HIV-1 syncytial plaques is counted as previously described (Kucera et al., AIDS Res. Human Retroviruses, 6: 491-501, (1990)).

### Cytotoxic Assay

The compounds described herein and AZT are assayed for cytotoxicity by inhibition of mouse precursor erythrocyte (CFU-E) and granulocyte/macrophage (CFU-GM) cell colony formation. Mouse bone marrow precursor cells are suspended in soft-agar-RPMI-1640 medium supplemented with cell growth factors and serial concentrations of the compound. After incubation at 37° C. to allow cell colony formation, the number of colony-forming units in the presence and absence of the compound is calculated to determine the inhibitory concentration for 50% (IC50).

### Inhibition of Retrovirus-Induced Splenomegaly

Mice are infected intra peritoneally with Friend-Leukemia virus and not treated or treated intra peritoneally with daily doses of compound. After 14 days infection and treatment, the mice are sacrificed; the spleens are removed and weighed to determine the percentage, of control splenomegaly. AZT is used as a control.

### Example 21

### Anti-HIV1 Activity

The inhibitory effects of the compounds and compositions described herein on the replication of human immunodeficiency virus type 1 (HIV-1) virus in cells can be examined by the plaque assay procedure of L. Kucera et al., Aids Research and Human Retroviruses 6, 491 (1990), or any procedure known to one of skill in the art. CEM-SS cell monolayers are infected with HIV-1. Infected cells are overlaid with RPMI-1640 plus 10% FBS supplemented with different concentrations of inhibitor. AZT and dideoxyinosine (DDI) can be used as positive controls. Plaques are counted at five days after infection. In this assay HIV-1 syncytial plaques are seen as large, multicellular foci (10 to 25 nuclei/syncytium) that appear either brown and granular or clear. Since the number of HIV-1 syncytial plaques correlates with reverse transcriptase (RT) and p24 core antigen activity in the HIV-1 infected cell overlay fluids, the syncytial plaque assay can be used to quantify the amount of infectious virus. Reverse transcriptase activity is assayed according to a described procedure (B. J. Poeisz et al., Proc. Natl. Acad. Sci. (U.S.A.) 77, 7415 (1980)). The activity of p24 core antigen induced by HIV-1 infection of CEM-SS cells is measured spectrophotometrically using the commercial Coulter EIA.

### Example 22

### Anti-HIV-1 Activity Over Time

The effect of compounds described herein on HIV-1 acutely infected H9 cells and persistently infected H9IIIB cells is evaluated by measuring reverse transcriptase (RT) and infectious virus production in supernatant fluids harvested at various times (days) after HIV-1 infection and continuous treatment with the compounds as described herein.

### Example 23

### Anti-HIV-1 Activity in Monocyte/Macrophages

Monocyte/macrophages represent a major reservoir of HIV-1 in the infected human host. See L. Epstein et al., AIDS Res. 14, 447 (1984). However, these cells tend to be resistant to dideoxynucleoside prodrugs due to low levels of kinases needed to activate the prodrugs. See C. Perno et al., J. Exp. Med. 168, 1111 (1984). The compounds and compositions described herein are assayed in HIV-1 persistently infected monocyte/macrophage (Ul) cells and the effect on HIV-1 replication is measured as described herein.

While illustrative embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A composition comprising:
i) an effective amount of a first compound of structure (C-I): 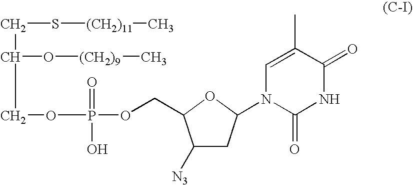
or a pharmaceutically acceptable salt thereof; and
ii) an effective amount of a second compound of structure (C-II), (C-III), (C-IV), (C-V) or (C-VI): 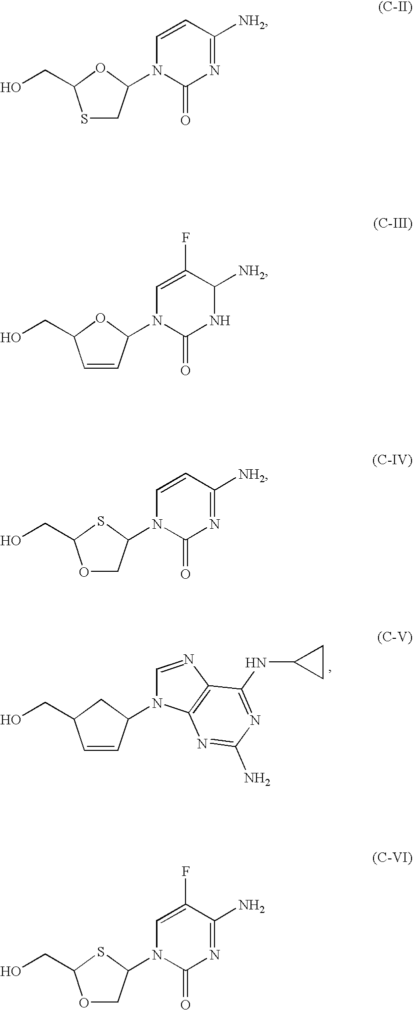
or a pharmaceutically acceptable salt thereof.

2. The composition of claim 1, further comprising a pharmaceutically acceptable carrier.

3. The composition of claim 1, wherein the first compound and the second compound are present in the composition in a molar ratio of about 0.1:1 to 10:1.

4. The composition of claim 1, wherein the first compound and the second compound are present in the composition in a ratio of about 10:1 to 2:1 by weight.

5. The composition of claim 1, wherein the first compound and the second compound are present in the composition in a ratio of about 3:1 to 2:1 by weight.

6. The composition of claim 1, wherein the first compound and the second compound are present in the composition in a ratio of about 3:1 by weight.

7. The composition of claim 1, comprising about 300-1000 mg of the first compound and about 50-500 mg of the second compound.

8. The composition of claim 1, comprising about 600-800 mg of the first compound and about 100-200 mg of the second compound.

9. The composition of claim 1, comprising about 600-800 mg of the first compound and about 150 mg of the second compound.

10. The composition of claim 1, wherein the second compound is a compound of structure (C-II), or a pharmaceutically acceptable salt thereof.

11. The composition of claim 10, wherein the second compound is a mixture of the cis-isomers of the compound of structure (C-II).

12. The composition of claim 10, wherein the second compound is a cis isomer of a compound of structure (C-II) in the form of a single enantiomer.

13. The composition of claim 12, wherein the second compound is the cis (−) isomer of a compound of structure (C-II).

14. The composition of claim 1, wherein the second compound is a compound of structure (C-III), or a pharmaceutically acceptable salt thereof.

15. The composition of claim 1, wherein the second compound is a compound of structure (C-IV), or a pharmaceutically acceptable salt thereof.

16. The composition of claim 15, wherein the second compound is a mixture of the cis-isomers of the compound of structure (C-IV).

17. The composition of claim 15, wherein the second compound is a cis isomer of a compound of structure (C-IV) in the form of a single enantiomer.

18. The composition of claim 17, wherein the second compound is the (1S,4R) isomer of a compound of structure (C-IV).

19. The composition of claim 1, wherein the second compound is a compound of structure (C-V), or a pharmaceutically acceptable salt thereof.

20. The composition of claim 19, wherein the second compound is a mixture of the cis-isomers of the compound of structure (C-V).

21. The composition of claim 19, wherein the second compound is a cis isomer of a compound of structure (C-V) in the form of a single enantiomer.

22. The composition of claim 21, wherein the second compound is the cis (−) isomer of a compound of structure (C-V).

23. The composition of claim 1, wherein the second compound is a compound of structure (C-VI), or a pharmaceutically acceptable salt thereof.

24. The composition of claim 23, wherein the second compound is a racemic mixture of the cis-isomers of a compound of structure (C-VI).

25. The composition of claim 23, wherein the second compound is a cis isomer of a compound of structure (C-VI) in the form of a single enantiomer.

26. The composition of claim 25, wherein the second compound is the cis (−) isomer of a compound of structure (C-VI).

27. A method for treating a viral disease comprising administering to a subject in need of thereof:
i) an effective amount of a first compound of structure (C-I): 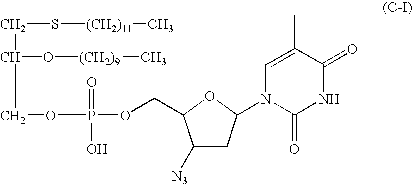
or a pharmaceutically acceptable salt thereof; and
ii) an effective amount of a second compound of structure (C-II), (C-III), (C-IV), (C-V) or (C-VI): 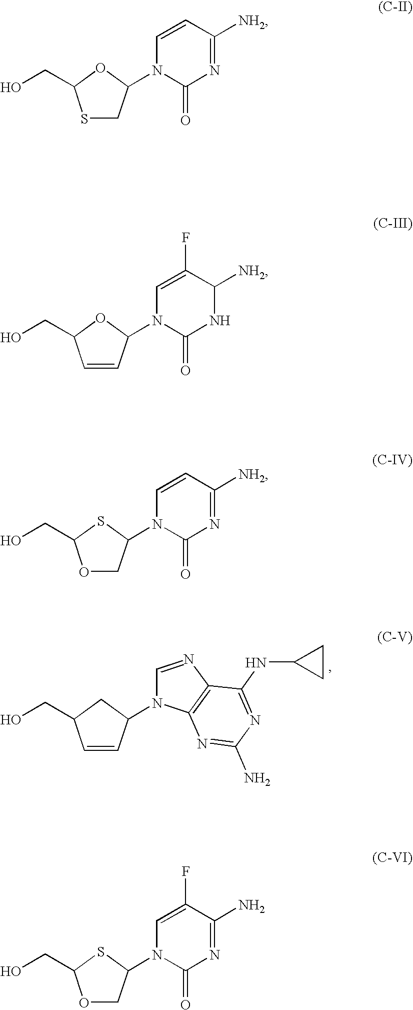
or a pharmaceutically acceptable salt thereof.

28. The method of claim 27, wherein the first compound and the second compound are present in a single composition.

29. The method of claim 27, wherein the first compound and the second compound are administered in a molar ratio of about 0.1:1 to 10:1.

30. The method of claim 27, wherein the first compound and the second compound are administered in a ratio of about 10:1 to 2:1 by weight.

31. The method of claim 27, wherein the first compound and the second compound are administered in a ratio of about 3:1 to 2:1 by weight.

32. The method of claim 27, wherein the first compound and the second compound are administered in a ratio of about 3:1 by weight.

33. The method of claim 27, wherein about 300-1000 mg of the first compound is administered and about 50-500 mg of the second compound is administered.

34. The method of claim 27, wherein about 600-800 mg of the first compound is administered and about 100-200 mg of the second compound is administered.

35. The method of claim 27, wherein about 600-800 mg of the first compound is administered and about 150 mg of the second compound is administered.

36. The method of claim 27, wherein the first compound and the second compound are administered sequentially.

37. The method of claim 27, wherein the first compound and the second compound are administered concurrently.

38. The method of claim 27, wherein the first compound and the second compound are administered one to four times per day.

39. The method of claim 27, wherein the first compound and the second compound are administered once to twice per day.

40. The method of claim 27, wherein the first compound and the second compound are administered once per day.

41. The method of claim 27, wherein the first compound and the second compound are administered two times a day.

42. The method of claim 27, wherein the viral disease is infection with a DNA virus, herpes simplex virus, cytomegalovirus, Papovavirus, varicella zoster virus, Epstein-Barr virus, an RNA viruse, togaviruse, a retroviruse, an oncovirus, HTLV-I., HTLV-II, a lentivirus, HIV-1, HIV-2 or chronic hepatitis B.

43. The method of claim 27, wherein the viral disease is HIV infection.

44. A composition comprising:
i) an effective amount of a first compound of formula (C-VII) or a pharmaceutically acceptable salt thereof: 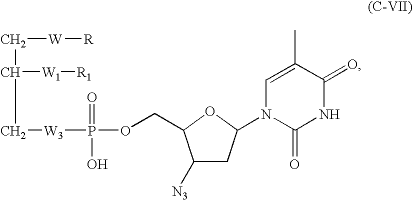
wherein
W is —S—, —S(O)—, —O—, —S—C═C—, —SC(O)—, —O—C═C— or —OC(O)—;
W₁ is —S—, —S(O)—, —O—, —S—C═C—, —SC(O)—, —O—C═C— or —OC(O)—;
W₃ is —O— or a covalent bond;
R is C1-C7, C8-C12 or C19-C24 cyclic, straight-chained or branched, unsaturated or saturated alkyl;
R₁ is C1, C2, C3-C10, C11-C15 or C18-C24 cyclic, straight-chained or branched, unsaturated or saturated alkyl; and
ii) an effective amount of a second compound of structure (C-II), (C-III), (C-IV), (C-V) or (C-VI): 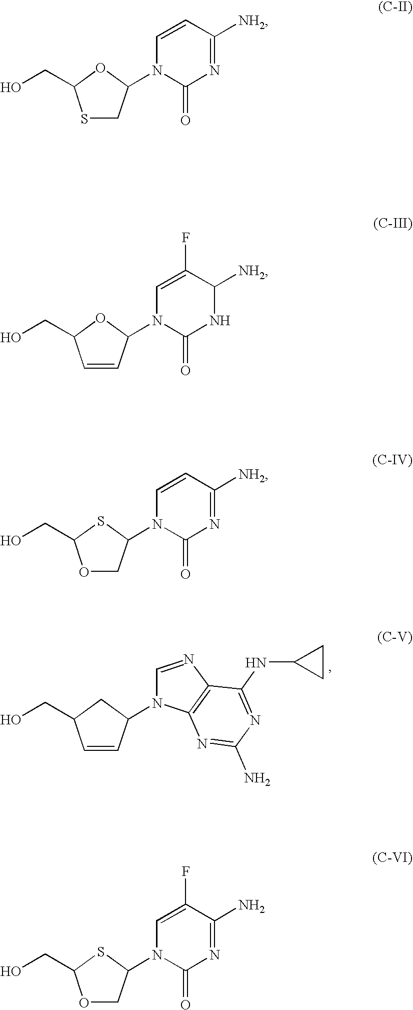
or a pharmaceutically acceptable salt thereof.

45. The composition of claim 44, wherein W is —S— or —S(O)—.

46. The composition of claim 44, wherein W is —O—.

47. The composition of claim 44, wherein R is optionally substituted with one or more phenyl, halogen, C₁-C₆-alkoxy, C₁-C₆-alkylmercapto, C₁-C₁-alkoxycarbonyl, C₁-C₆-alkylsulphinyl or C₁-C₆-alkylsulphonyl groups.

48. The composition of claim 44, wherein R₁ is optionally substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonyl groups.

49. The composition of claim 44, wherein R is a C8-C12 straight-chained or branched, unsaturated or saturated alkyl.

50. The composition of claim 49, wherein R is optionally substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonyl groups.

51. The composition of claim 49, wherein R is a C12 straight-chained or branched, unsaturated or saturated alkyl.

52. The composition of claim 51, wherein R is optionally substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonyl groups.

53. The composition of claim 49, wherein R is a straight-chained, saturated alkyl.

54. The composition of claim 44, wherein R₁ is a C3-C7 alkyl.

55. The composition of claim 44, wherein R₁ is a C2 alkyl.

56. The composition of claim 44, wherein the first compound is a compound of structure (C-I).

57. The composition of claim 44, wherein the second compound is a compound of structure (C-II).

58. The composition of claim 44, wherein the second compound is a compound of structure (C-III).

59. The composition of claim 44, wherein the second compound is a compound of structure (C-IV).

60. The composition of claim 44, wherein the second compound is a compound of structure (C-V).

61. The composition of claim 44, wherein the second compound is a compound of structure (C-VI).

62. A method for treating a viral disease comprising administering to a subject in need of thereof:
i) an effective amount of a first compound of formula (C-VII): 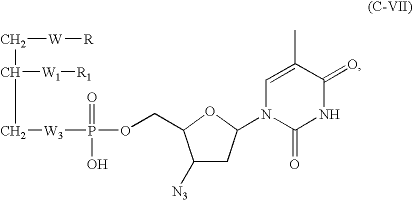
wherein
W is —S—, —S(O)—, —O—, —S—C═C—, —SC(O)—, —O—C═C— or —OC(O)—;
W₁ is —S—, —S(O)—, —O—, —S—C═C—, —SC(O)—, —O—C═C— or —OC(O)—;
W₃ is —O— or a covalent bond;
R is C1-C7, C8-C12 or C19-C24 cyclic, straight-chained or branched, unsaturated or saturated alkyl;
R₁ is C1, C2, C3-C10, C11-C15 or C18-C24 cyclic, straight-chained or branched, unsaturated or saturated alkyl; and
ii) an effective amount of a second compound of structure (C-II), (C-III), (C-IV), (C-V) or (C-VI): 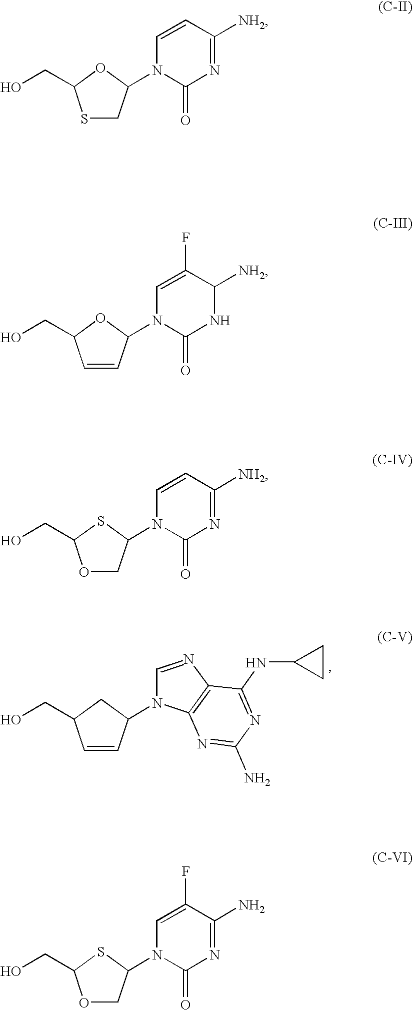
or a pharmaceutically acceptable salt thereof.

63. The method of claim 61, wherein W is —S— or —S(O)—.

64. The method of claim 61, wherein W₁ is —O—.

65. The method of claim 61, wherein R is optionally substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonyl groups.

66. The method of claim 61, wherein R₁ is optionally substituted with one or more phenyl, halogen, C₁-C₆-alkoxy, C₁-C₆-alkylmercapto, C₁-C₆-alkoxycarbonyl, C₁-C₆-alkylsulphinyl or C₁-C₆-alkylsulphonyl groups

67. The method of claim 61, wherein the first compound and the second compound are present in a single composition.

68. The method of claim 61, wherein the first compound and the second compound are administered sequentially.

69. The method of claim 61, wherein the first compound and the second compound are administered concurrently.

70. The method of claim 61, wherein the first compound and the second compound are administered one to four times per day.

71. The method of claim 61, wherein the first compound and the second compound are administered once to twice per day

72. The method of claim 61, wherein the first compound and the second compound are administered once per day.

73. The method of claim 61, wherein the first compound and the second compound are administered two times a day.

74. The method of claim 61, wherein the viral disease is infection with a DNA virus, herpes simplex virus, cytomegalovirus, Papovavirus, varicella zoster virus, Epstein-Barr virus, an RNA viruse, togaviruse, a retroviruse, an oncovirus, HTLV-I., HTLV-II, a lentivirus, HIV-1, HIV-2 or chronic hepatitis B.

75. The method of claim 61, wherein the viral disease is HIV infection.
